(19) United States
(12) Patent Application Publication
(10) Pub. No.: US 2005/0152905 A1
(43) Pub. Date: Jul. 14, 2005

(54) Method of biochemical treatment of persistent pain

(76) Inventor: Osemwota Sota Omoigui, Tarzana, CA (US)

Correspondence Address: Osemwota Sota Omoigui, 4019 W. Rosecrans Ave, Hawthorne, CA, 90250, US

(21) Appl. No.: 11/058,371

(22) Filed: Feb. 16, 2005

Related U.S. Application Data
(63) Continuation-in-part of application No. 10/224,743, filed on Aug. 22, 2002.

(51) Int. Cl. A61K 39/395; A61K 31/675; A61K 31/66; A61K 31/573

(52) U.S. Cl. 424/145.1; 514/12; 514/102; 514/89; 514/154; 514/263.31; 514/171; 424/239.1; 514/569; 514/570; 514/420; 514/406; 514/217; 514/23; 514/561; 514/557; 514/355; 514/301; 514/282

(57) ABSTRACT

This invention relates to a method for the biochemical treatment of persistent pain disorders by inhibiting the biochemical mediators of inflammation in a subject comprising administering to said subject any one of several combinations of components that are inhibitors of biochemical mediators of inflammation. Said process for biochemical treatment of persistent pain disorders is based on Sota Omoigui's Law, which states: ‘The origin of all pain is inflammation and the inflammatory response’. Sota Omoigui's Law of Pain unifies all pain syndromes as sharing a common origin of inflammation and the inflammatory response. The various biochemical mediators of inflammation are present in differing amounts in all pain syndromes and are responsible for the pain experience. Classification and treatment of pain syndromes should depend on the complex inflammatory profile. A variety of mediators are generated by tissue injury and inflammation. These include substances produced by damaged tissue, substances of vascular origin as well as substances released by nerve fibers themselves, sympathetic fibers and various immune cells. Biochemical mediators of inflammation that are targeted for inhibition include but are not limited to: prostaglandin, nitric oxide, tumor necrosis factor alpha, interleukin 1-alpha, interleukin 1-beta, interleukin-4, Interleukin-6 and interleukin-8, histamine and serotonin, substance P, Matrix Metallo-Proteinase, calcitonin gene-related peptide, vasoactive intestinal peptide as well as the potent inflammatory mediator peptide proteins neurokinin A, bradykinin, kallidin and T-kinin.

### BACKGROUND OF THE INVENTION

This invention relates to a method of biochemical treatment of persistent pain by application of Sota Omoigui's Law, which states: The origin of all pain is inflammation and the inflammatory response. Irrespective of the type of pain whether it is acute pain as in a sprain, sports injury or eurochange jellyfish sting or whether it is chronic pain as in arthritis, migraine pain, back or neck pain from herniated disks, RSD/CRPS pain, migraine, Fibromyalgia, Interstitial cystitis, Neuropathic pain, Post-stroke pain etc, the underlying basis is inflammation and the inflammatory response. Irrespective of the characteristic of the pain, whether it is sharp, dull, aching, burning, stabbing, numbing or tingling, all pain arise from inflammation and the inflammatory response.

### DESCRIPTION OF THE PRIOR ART

PRIOR ART—The Prior Art does not contain any unifying Law of Pain such as Sota Omoigui's Law of Pain. Each disease entity e.g. rheumatoid arthritis and ankylosing spondylitis is considered distinct from the other entities and is classified in terms of structural pathology, genetic markers, and presence of autoantibodies. Where present, treatment of inflammation in these disease entities has hitherto addressed one biochemical mediator of inflammation at a time, instead of addressing the inflammatory soup of biochemical mediators that are present in all pain syndromes. Sota Omoigui's Law of Pain unifies all pain syndromes as sharing a common origin of inflammation and the inflammatory response. The various biochemical mediators of inflammation are present in differing amounts in all pain syndromes and are responsible for the pain experience. Classification and treatment of pain syndromes should depend on the complex inflammatory profile and should not be based alone on structural pathology, genetic markers or presence of autoantibodies.

Four centuries ago Descarte described pain in terms of an alarm bell ringing in a bell tower. In 1898, in his landmark work, The Integrative Action of the Nervous System¹, the British physiologist, Sir Charles Scott Sherrington, proposed the key concept of nociception: pain as the evolved response to a potentially harmful, “noxious” stimulus. Livingston wrote in his Pain Mechanisms²: “I believe that the concept of ‘specificity’ in the narrow sense in which it is sometimes used has led away from a true perspective. Pain is a sensory experience that is subjective and individual; it frequently exceeds its protective function and becomes destructive. The impulses, which subserve it, are not pain, but merely a part of its underlying and alterable physical mechanisms. The specificity of function of neuron units cannot be safely transposed into terms of sensory experience. A chronic irritation of sensory nerves may initiate clinical states that are characterized by pain and a spreading disturbance of function in both somatic and visceral structures. If such disturbances are permitted to continue, profound and perhaps unalterable organic changes may result in the affected part. A vicious circle is thus created.”³ In 1965, collaboration between Canadian psychologist Ronald Melzack and British physiologist Patrick Wall produced the gate control theory. Their paper, “Pain Mechanisms: A New Theory,⁴ has previously been described as “the most influential ever written in the field of pain.” Melzack and Wall suggested a gating mechanism within the spinal cord that closed in response to normal stimulation of the fast conducting “touch” nerve fibers; but opened when the slow conducting “pain” fibers transmitted a high volume and intensity of sensory signals. The gate could be closed again if these signals were countered by renewed stimulation of the large fibers. Pain is currently defined by the International Association for the Study of Pain (IASP) as ‘an unpleasant sensory or emotional experience associated with actual or potential tissue damage, or described in terms of such damage’. This definition was adapted in 1979, and published in the paper ‘Pain terms; a list with definitions and notes on usage. Recommended by the IASP Subcommittee on Taxonomy’, in the journal Pain in 1979⁵. This definition was subsequently considered elusive, and the following statement was added in order to make the position more clear: ‘Pain is always subjective. Each individual learns the application of the word through experiences related to injury in early life. It is unquestionably a sensation in a part of the body but it is also unpleasant and therefore also an emotional experience. Many people report pain in the absence of tissue damage or any likely pathophysiological cause; usually this happens for psychological reasons. There is no way to distinguish their experience from that due to tissue damage, if we take the subjective report. If they regard their experience as pain and if they report it in the same ways as pain caused by tissue damage, it should be accepted as pain. This definition avoids tying pain to the stimulus . . . . ’ Pain is also currently classified as being peripheral or central in origin. Peripheral pain originates in muscles, tendons, etc., or in the peripheral nerves. Pain originating in the peripheral nerves, i.e. via trauma to the nerves, is neurogenic pain. Central pain arises from central nervous system pathology: a “primary” CNS dysfunction. Some of this has been thought to arise due to maladaptive thought processes, true “psychogenic” pain⁶. But most of it has been thought to be due to structural changes in the CNS, e.g., spinal cord injury, multiple sclerosis, stroke and epilepsy (Boivie, 1996)^{7 8}.

Another current classification, that distinguishes between normally functioning nerves and nerves whose function has been altered by pathology is as follows: Nociceptive pain is pain in which normal nerves transmit information to the central nervous system about trauma to tissues. Neuropathic pain is pain in which there are structural and/or functional nervous system adaptations secondary to injury, that take place either centrally or peripherally (Jensen, 1996)⁹. The IASP defines central pain as “pain initiated or caused by a primary lesion or dysfunction in the central nervous system” (Merskey, and Bogduk, 1994)¹⁰. Current medical theories place an over reliance on structural abnormalities to explain pain syndromes. This is not surprising because our current imaging technologies are structure based. Physicians are comfortable treating what they see. Patients who have structural abnormalities such as a osteoarthritis or herniated disk on MRI scans get operated upon often times needlessly and end up with more joint, back or neck pain. Patients with severe pain who do not have structural abnormalities on MRI scans are dismissed as psychiatric cases. The fallacy of this approach has been confirmed in numerous published studies. In one of these studies¹¹, the authors performed magnetic resonance imaging on sixty-seven individuals who had never had low-back pain, sciatica, or neurogenic claudication. The scans were interpreted independently by three neuro-radiologists who had no knowledge about the presence or absence of clinical symptoms in the subjects. About one-third of the subjects were found to have a substantial abnormality. Of those who were less than sixty years old, 20 percent had a herniated nucleus pulposus and one had spinal stenosis. In the group that was sixty years old or older, the findings were abnormal on about 57 percent of the scans: 36 percent of the subjects had a herniated nucleus pulposus and 21 percent had spinal stenosis. There was degeneration or bulging of a disc at least one lumbar level in 35 percent of the subjects between twenty and thirty-nine years old and in all but one of the sixty to eighty-year-old subjects. In view of these findings in asymptomatic subjects, the authors concluded that abnormalities on magnetic resonance images must be strictly correlated with age and any clinical signs and symptoms before operative treatment is contemplated. In another study¹², the authors examined the prevalence of abnormal findings on magnetic resonance imaging (MRI) scans of the lumbar spine in people without back pain. 52 percent of the asymptomatic subjects were found to have a bulge at least at one level, 27 percent had a protrusion, and 1 percent had an extrusion. Thirty-eight percent had an abnormality of more than one intervertebral disk. The prevalence of bulges, but not of protrusions, increased with age. The most common nonintervertebral disk abnormalities were Schmorl's nodes (herniation of the disk into the vertebral-body end plate), found in 19 percent of the subjects; annular defects (disruption of the outer fibrous ring of the disk), in 14 percent; and facet arthropathy (degenerative disease of the posterior articular processes of the vertebrae), in 8 percent. The findings were similar in men and women. The authors concluded that on MRI examination of the lumbar spine, many people without back pain have disk bulges or protrusions but not extrusions. The authors went further to state that given the high prevalence of these findings and of back pain, the discovery by MRI of bulges or protrusions in people with low back pain may frequently be coincidental. In another study¹³, which tracked the natural history of individuals with asymptomatic disc abnormalities in magnetic resonance imaging the authors stated that the high rate of lumbar disc alterations recently detected in asymptomatic individuals by magnetic resonance imaging demands reconsideration of a pathomorphology-based explanation of low back pain and sciatica. In another controlled trial of arthroscopic surgery for osteoarthritis of the knee¹⁴, 180 patients with osteoarthritis of the knee were randomly assigned to receive arthroscopic debridement, arthroscopic lavage, or placebo surgery. Patients in the placebo group received skin incisions and underwent a simulated debridement without insertion of the arthroscope. Patients and assessors of outcome were blinded to the treatment-group assignment. Outcomes were assessed at multiple points over a 24-month period with the use of five self-reported scores—three on scales for pain and two on scales for function—and one objective test of walking and stair climbing. A total of 165 patients completed the trial. The study results were astounding. At no point did either of the intervention groups report less pain or better function than the placebo group. For example, mean (±SD) scores on the Knee-Specific Pain Scale (range, 0 to 100, with higher scores indicating more severe pain) were similar in the placebo, lavage, and debridement groups: 48.9±21.9, 54.8±19.8, and 51.7±22.4, respectively, at one year (P=0.14 for the comparison between placebo and lavage; P=0.51 for the comparison between placebo and debridement) and 51.6±23.7, 53.7±23.7, and 51.4±23.2, respectively, at two years (P=0.64 and P=0.96, respectively). Furthermore, the 95 percent confidence intervals for the differences between the placebo group and the intervention groups exclude any clinically meaningful difference. The authors concluded that in this controlled trial involving patients with osteoarthritis of the knee, the outcomes after arthroscopic lavage or arthroscopic debridement were no better than those after a placebo procedure. This is further confirmation of the fallacy of a structure-based approach to the treatment of pain.

### SUMMARY OF THE INVENTION

The present invention provides a method for the biochemical treatment of persistent pain in a human by the use of combinations of drugs or medication that antagonize the biochemical mediators of inflammation. Sota Omoigui's Law of Pain states that: The origin of all pain is inflammation and the inflammatory response. Sota Omoigui's Law of Pain unifies all pain syndromes as sharing a common origin of inflammation and the inflammatory response. The various biochemical mediators of inflammation are present in differing amounts in all pain syndromes and are responsible for the pain experience. Irrespective of the type of pain whether it is acute pain as in a sprain, sports injury or eurochange jellyfish sting or whether it is chronic pain as in arthritis, migraine, back or neck pain from herniated disks, RSD/CRPS pain, Fibromyalgia, Interstitial cystitis, Neuropathic pain, Post-stroke pain etc, the underlying basis is inflammation and the inflammatory response. Irrespective of the characteristic of the pain, whether it is sharp, dull, aching, burning, stabbing, numbing or tingling, all pain arise from inflammation and the inflammatory response. On the basis of Sota Omoigui's Law of Pain, antagonism of inflammation and the inflammatory response will relieve pain of every origin, type and character.

The biochemical mediators produced by the immune cells include prostaglandin, nitric oxide, tumor necrosis factor alpha, interleukin 1-alpha, interleukin 1-beta, interleukin-4, Interleukin-6 and interleukin-8, histamine, serotonin. The biochemical mediators produced by the nerve cells include inflammatory protein Substance P, calcitonin gene-related peptide (CGRP) neurokinin A and vasoactive intestinal peptide.

Cell enzymes that catalyze reaction pathways and generate these biochemical mediators of inflammation include cyclooxygenase (COX), lipoxygenase (LOX). A cell enzyme that is activated by inflammatory mediators such as TNF-alpha and interleukin-1 is Gelatinase B or Matrix Metallo-Proteinase 9 (MMP-9). Once activated MMP-9 helps immune cells migrate through the blood vessels to inflammatory sites or to metastatic sites. Activated, MMP-9 can also degrade collagen in the extra cellular matrix of articular bone and cartilage and is associated with joint inflammation and bony erosions¹⁵.

Drugs and medications, which inhibit these biochemical mediators of inflammation, include:

Non-steroidal anti-inflammatories, such as aspirin, tolmetin sodium, indomethacin and ibuprofen. These medications inhibit the enzyme cyclooxygenase and therefore decrease prostaglandin synthesis. Prostaglandins are inflammatory mediators that are released during allergic and inflammatory processes. Phospholipase A2 enzyme, which is present in cell membranes, is stimulated or activated by tissue injury or microbial products. Activation of phospholipase A2 causes the release of arachidonic acid from the cell membrane phospholipid. From here there are two reaction pathways that are catalyzed by the enzymes cyclooxygenase and lipoxygenase. The cyclooxygenase enzyme pathway results in the formation of inflammatory mediator prostaglandins and thromboxane.

Glucocorticoids are naturally occurring hormones that prevent or suppress inflammation and immune responses when administered at pharmacological doses. The anti-inflammatory corticosteroids inhibit the activation of phospholipase A₂ by causing the synthesis of an inhibitory protein called lipocortin. It is lipocortin that inhibits the activity of phospholipases and therefore limits the production of potent mediators of inflammation such as prostaglandins and leukotriene.

Botulinum toxins are potent neurotoxins which block the release of neurotransmitters. One of these transmitters called acetylcholine is released by nerve cells and transported into muscle cells to signal the muscle to contract. Blockade of this transmitter by Botulinum toxin can produce a long lasting relief of muscle spasms. Botulinum toxins also inhibit the release of tumor necrosis factor alpha¹⁶ (TNF-alpha) from immune cells and thus can alleviate pain and spasm produced by the inflammatory response.

Tumor Necrosis Factor Alpha Blocker Medications

The central role in inflammatory responses have Interleukin-1 and TNF-alpha, because the administration of their antagonists, such as IL-1ra (Interleukin-1 receptor antagonist), soluble fragment of Interleukin-1 receptor, or monoclonal antibodies to TNF-alpha and soluble TNF receptor, all block various acute and chronic responses in animal models of inflammatory diseases.

Etanercept (ENBREL) is a fusion protein produced by recombinant DNA technology. Etanercept binds to and inactivates Tumor Necrosis Factor (TNF-alpha) but does not affect TNF-alpha production or serum levels. Etanercept may also modulate other biologic responses that are induced or regulated by TNF-alpha such as production of adhesion molecules, other inflammatory cytokines and matrix metalloproteinase-3 (MMP-3 or stromelysin).

Infliximab is a monoclonal antibody targeted against tumor necrosis factor-alpha (TNF-alpha). Infliximab neutralizes the biological activity of the cytokine tumor necrosis factor-alpha (TNF-alpha). Infliximab binds to high affinity soluble and transmembrane forms of TNF-alpha and inhibits the binding of TNF-alpha with its receptors. Infliximab does not neutralize TNF-beta, a related cytokine that utilizes the same receptors as TNF-alpha. Biological activities attributed to TNF-alpha include induction of pro-inflammatory cytokines such as interleukin (IL)-1 and IL-6; enhancement of leukocyte migration by increasing endothelial layer permeability; expression of adhesion molecules by endothelial cells and leukocytes; activation of neutrophil and eosinophil functional activity; fibroblast proliferation; synthesis of prostaglandins; and induction of acute phase and other liver proteins.

Anakinra is a form of the human interleukin-1 receptor antagonist (IL-1Ra) produced by recombinant DNA technology. Anakinra differs from the naturally occurring native human IL-1Ra in that it has an additional methionine residue at its amino terminus. Anakinra acts similarly to the naturally occurring interleukin-1 receptor antagonist (IL-1Ra). IL-1Ra blocks effects of Interleukin-1 by competitively inhibiting binding of this cytokine, specifically IL-alpha and IL-beta, to the interleukin-1 type 1 receptor (IL-1R1), which is produced in a wide variety of tissues. Il-1Ra is part of the feedback loop that is designed to balance the effects of inflammatory cytokines.

Leflunomide interferes with RNA and protein synthesis in immune T and B-lymphocytes. T and B cell collaborative actions are interrupted and antibody production is suppressed. Leflunomide is the first agent for rheumatoid arthritis that is indicated for both symptomatic improvement and retardation of structural joint damage. Leflunomide may also have anti-inflammatory properties secondary to reduction of histamine release, and inhibition of induction of cyclooxygenase-2 enzyme (COX-2). Leflunomide may decrease proliferation, aggregation and adhesion of peripheral and joint fluid mononuclear cells. Decrease in the activity of immune lymphocytes leads to reduced cytokine and antibody-mediated destruction of joints and attenuation of the inflammatory process.

Phosphodiesterase inhibitors such as Pentoxifylline have other unique effects. The drugs suppress inflammatory cytokine production by T cells and macrophages¹⁷. Some of the anti-inflammatory effects occurs by blocking nitric oxide (NO) production by macrophages.

Pentoxifylline also blocks the production of Tumor Necrosis Factor Alpha. In one study, Pentoxifylline prevented nerve root injury and swelling (dorsal root ganglion compartment syndrome) caused by topical application of disk tissue (nucleus pulposus)¹⁸

Tetracyclines such as doxycycline and minocycline may block a number of cytokines including Interleukin-1 ¹⁹²⁰, IFNg²¹, NO-synthetases, and metalloproteinases²². Interleukin-1 and IFN-.gamma act synergistically with TNF-alpha and are known to be toxic to nerve tissue^{23 24252627}.

5-HT3-receptor antagonist medications such as Ondansetron diminish serotonin-induced release of substance P from C-fibers and prevent unmasking of NK2-receptors in the presence of serotonin²⁸.

Bisphosphonates medications such as Pamidronate reduce bone complications and related pain in patients with Paget's disease, osteoporosis and bone metastasis, thereby improving quality of life. Bisphosphonates have intrinsic anti-tumor activity by virtue of inducing tumor cell adherence to marrow, reducing interleukin-6 secretion, inducing tumor cell apoptosis, or inhibiting angiogenesis²⁹

Anti-depressant medication such as Amitriptyline also have effects on inflammatory mediators. Prolonged administration of amitriptyline and desipramine have resulted in a significant increase in the secretion of the anti-inflammatory cytokine Interleukin-10³⁰.

Anti-seizure medications such as Oxcarbazepine or Zonisamide modulate neuronal transmission and inhibit neuronal neuropeptide (Substance P, Glutamate, Calcitonin Gene Related Peptide, Bradykinin, Nitric Oxide) release. These medications decrease pain by reducing the rate of continuing discharge of injured and inflamed nerve fibers. Blockade of sodium channels in nerve cells leads to a decrease in electrical activity and a subsequent reduction in release of the excitatory nerve transmitter glutamate. Anti-seizure drugs also inhibit the initiation and propagation of painful nerve impulses by inhibiting Nitric Oxide Synthetase activity³¹. Nitric Oxide Synthetase is the enzyme responsible for the production of the inflammatory mediator Nitric Oxide. Anti-seizure drugs may also protect nerve cells from free radical damage by Nitric Oxide and/or hydroxyl radicals (OH*)³²

Thalidomide and analogues mainly inhibit tumor necrosis factor alpha (TNF-alpha) synthesis but the drugs also have effects on other cytokines. Thalidomides increase the production of the anti-inflammatory cytokine interleukin-10 (IL-10) in lesioned sciatic nerves. In addition, Thalidomides stimulate the release of the pain relieving natural opioid peptide methionine-enkephalin in the dorsal horn of the spinal cord³³

### DETAILED DESCRIPTION

The origins of pain are the biochemical mediators of inflammation and the inflammatory response. To treat pain, we must block these mediators and block the signals they send up through the nerve cells. We can now measure many of these inflammatory mediators in the blood and spinal fluid. However, our current technology does not allow us to image these mediators. Hopefully sometime in the future we will be able to do so.

Inflammation occurs when there is infection or tissue injury. Tissue injury may arise from a physical, chemical or biological trauma or irritation. Degeneration of tissue subsequent to aging or previous injury can also lead to inflammation. Injured tissues can be muscle, ligament, disks, joints or nerves. A variety of mediators are generated by tissue injury and inflammation. These include substances produced by damaged tissue, substances of vascular origin as well as substances released by nerve fibers themselves, sympathetic fibers and various immune cells³⁴. There are three phases of an inflammatory response: initiation, maintenance and termination. Upon tissue injury or painful stimulation, specialized blood cells in the area such as basophils, mast cells and platelets release inflammatory mediators serotonin, histamine and nitric oxide. Subsequent to the binding of serotonin to its receptor, there is inflammation of the adjacent nerves and the nerve endings release short-lived inflammatory peptide proteins such as substance P, Calcitonin gene-related peptide (CGRP). In addition, clotting factors in the blood produce and activate potent inflammatory mediator peptide proteins called neurokinin A, bradykinin, kallidin and T-kinin. All of these proteins increase blood flow to the area of injury, stimulate arachidonic acid metabolism to generate inflammatory mediators prostaglandins and attract specialized immune cells to the area. The first immune cells to the area are tissue macrophages, which provide the front line defense against bacterial infection. Macrophages release powerful enzymes to digest any bacteria that are present and produce potent inflammatory chemical mediators (called cytokines) to attract and activate other cells of the immune system. Shortly thereafter the area of bacterial invasion or tissue injury is invaded by the other immune cells, which include white blood cells such as T helper cells, lymphocytes, neutrophils, eosinophils, and other cells such as fibroblasts and endothelial cells. These immune cells respond to the chemical mediators, release destructive enzymes to kill any invading organism and release more chemical mediators to attract more immune cells. A consequence of this immune response is tissue damage, pain and spasm. In a sense the initial immune reaction ignites a cascade of immune reactions and generates an inflammatory soup of chemical mediators. These chemical mediators produced by the immune cells include prostaglandin, nitric oxide, tumor necrosis factor alpha, interleukin 1-alpha, interleukin 1-beta, interleukin-4, Interleukin-6 and interleukin-8, histamine, serotonin, In the area of injury and subsequently in the spinal cord, enzymes such as cyclooxygenase increase the production of these inflammatory mediators. These chemical mediators attract tissue macrophages and white blood cells to localize in an area to engulf (phagocytize) and destroy foreign substances. The chemical mediators released during the inflammatory response give rise to the typical findings associated with inflammation.

Effects of the Inflammatory Response.

The primary physical effect of the inflammatory response is for blood circulation to increase around the affected area. Blood vessels around the site of inflammation dilate, allowing increased blood flow to the area. Gaps appear in the cell walls surrounding the area, allowing the larger cells of the blood, i.e. the immune cells, to pass through. As a result of the increased blood flow, the immune presence is increased. All of the different types of cells that constitute the immune system congregate at the site of inflammation, along with a large supply of chemical mediators, which fuel the immune response. There is an increase in local or body heat. The main symptoms of the inflammatory response are as follows.

1. The tissues in the area are red and warm, as a result of the large amount of blood reaching the site.

2. The tissues in the area are swollen, again due to the increased amount of blood and proteins that are present.

3. The tissues in the area are painful, due to the presence of the inflammatory mediators and due to the expansion of tissues, causing mechanical pressure on nerve cells.

Effects of the Inflammatory Mediators

The inflammatory mediators activate local pain receptors and nerve terminals and produce hypersensitivity in the area of injury. Activity of the mediators results in excitation of pain receptors in the skin, ligaments, muscle, nerves and joints. Excitation of these pain receptors stimulate the specialized nerves e.g. C fibers and A-delta fibers that carry pain impulses to the spinal cord and brain. Subsequent to tissue injury, the expression of sodium channels in nerve fibers is altered significantly thus leading to abnormal excitability in the sensory neurons. Nerve impulses arriving in the spinal cord stimulate the release of inflammatory protein Substance P. The presence of Substance P and other inflammatory proteins such as calcitonin gene-related peptide (CGRP) neurokinin A and vasoactive intestinal peptide removes magnesium induced inhibition and enables excitatory Inflammatory proteins such as glutamate and aspartate to activate specialized spinal cord NMDA receptors. This results in magnification of all nerve traffic and pain stimuli that arrive in the spinal cord from the periphery. Activation of motor nerves that travel from the spinal cord to the muscles results in excessive muscle tension. More inflammatory mediators are released which then excite additional pain receptors in muscles, tendons and joints generating more nerve traffic and increased muscle spasm. Persistent abnormal spinal reflex transmission due to local injury or even inappropriate postural habits may then result in a vicious circle between muscle hypertension and pain³⁵. Separately, constant C-fiber nerve stimulation to transmission pathways in the spinal cord resulting in even more release of inflammatory mediators but this time within the spinal cord. Inflammation causes increased production of the enzyme cyclooxygenase-2 (Cox-2), leading to the release of chemical mediators both in the area of injury and in the spinal cord. Widespread induction of Cox-2 expression in spinal cord neurons and in other regions of the central nervous system elevates inflammatory mediator prostaglandin E₂ (PGE₂) levels in the cerebrospinal fluid. The major inducer of central Cox-2 upregulation is inflammatory mediator interleukin-1^{β} in the CNS³⁶. Basal levels of the enzyme phospholipase A₂ activity in the CNS do not change with peripheral inflammation. Abnormal development of sensory-sympathetic connections follow nerve injury, and contribute to the hyperalgesia (abnormally severe pain) and allodynia (pain due to normally innocuous stimuli). These abnormal connections between sympathetic and sensory neurons arise in part due to sprouting of sympathetic axons. Studies have shown that sympathetic axons invade spinal cord dorsal root ganglia (DRG) following nerve injury, and activity in the resulting pericellular axonal ‘baskets’ may underlie painful sympathetic-sensory coupling³⁷. Sympathetic sprouting into the DRG may be stimulated by neurotrophins such as nerve growth factor (NGF), brain derived neurotrophic factor (BDNF), neurotrophin-3 (NT-3) and neurotrophin 4/5 (NT-4/5). The central nervous system response to pain can keep increasing even though the painful stimulus from the injured tissue remains steady. This “wind-up” phenomenon in deep dorsal neurons can dramatically increase the injured person's sensitivity to the pain. Local tissue inflammation can also result in pain hypersensitivity in neighboring uninjured tissue (secondary hyperalgesia) by spread and diffusion of the excess inflammatory mediators that have been produced as well as by an increase in nerve excitability in the spinal cord (central sensitization). This can result in a syndrome comprising diffuse muscle pain and spasm, joint pain, fever, lethargy and anorexia.

The Complex interaction of Inflammatory Mediators

The inflammatory mediators interact in a complex way to induce, enhance and propagate persistent pain. There are also natural anti-inflammatory mediators produced by the body to cool down inflammation and the inflammatory response.

Interleukin-1 beta is a potent pain-generating mediator. Two pain producing pathways have been identified: Inflammatory stimuli or injury to soft tissue induces the production of mediator Bradykinin, which stimulates the release of mediator Tumor necrosis factor alpha. The TNF-alpha induces production of (i) Interleukin-6 and Interleukin-1-Beta which stimulate the production of cyclooxygenase enzyme products, and (ii) Inflammatory mediator Interleukin -8, which stimulates production of sympathomimetics (sympathetic hyperalgesia)³⁸. Effects of Interleukin-1 beta include:

Interleukin-1 beta stimulates inflammatory mediators prostaglandin E₂ (PGE₂), cyclooxygenase-2 (COX-2) and matrix metalloproteases (MMPs) production^{39,40}

Interleukin-1 beta is a significant catalyst in cartilage damage. It induces the loss of proteoglycans, prevents the formation of the cartilage matrix⁴¹ and prevents the proper maintenance of cartilage.

Interleukin -1 beta is a significant catalyst in bone resorption. It stimulates osteoclasts cells involved in the resorption and removal of bone⁴²⁴³⁴⁴

Interleukin-6

This is another potent pain-generating inflammatory mediator. IL-6 is one of a family of cytokines collectively termed “the interleukin-6-type cytokines”. The cytokines which make up this family are IL-6, leukemia inhibitory factor (LIF), oncostatin-M (OSM), ciliary neurotrophic factor (CNTF), cardiotrophin-1 (CT-1), and interleukin-11^{45 46}. IL-6 is involved in a myriad of biologic processes, perhaps explaining its long list of synonyms (B-cell stimulatory factor-2, B cell differentiation factor, T cell-replacing factor, interferon-β₂, 26-kDa protein, hybridoma growth factor, interleukin hybridoma plasmacytoma factor 1, plasmacytoma growth factor, hepatocyte-stimulating factor, macrophage granulocyte-inducing factor 2, cytotoxic T cell differentiation factor, thrombopoietin)⁴⁷. Although a normal physiologic process, aging is accompanied by a variety of disorders⁴⁸ including, Osteoarthritis, Alzheimer's disease, arteriosclerosis, and thyroiditis⁴⁹. IL-6 levels are directly correlated with aging in a variety of species⁵⁰, and it plays an important role in the aging process. Intriguingly, dietary restriction, the only experimental intervention that reproducibly prolongs maximum lifespan in mammals⁵¹ can restore to the young phenotype a variety of physiologic parameters, including IL-6 secretion and serum levels^{52 53}. Similarly, DHEA, currently thought to influence various aging processes⁵⁴, also has been shown to diminish the age-associated rise in serum IL-6⁵⁵. Among its many functions, IL-6 plays an active role in inflammation, immunology, bone metabolism, reproduction, arthritis, neoplasia, and aging. IL-6 expression is regulated by a variety of factors, including steroidal hormones, at both the transcriptional and post-transcriptional levels. IL-6 achieves its effects through the ligand-specific IL-6 receptor (IL-6R). Unlike most other cytokine receptors, the IL-6R is active in both membrane bound and soluble forms. IL-6 induces differentiation of activated, but not resting, B cells⁵⁶ culminating in production of immunoglobulin. Along with B cell differentiation, IL-6 stimulates proliferation of thymic and peripheral T cells⁵⁷ and in cooperation with IL-1⁵⁸, induces T cell differentiation to cytolytic-T cells^{59 60}and activates natural killer cells⁶¹. IL-6 appears to play an important role in bone metabolism through induction of osteoclastogenesis and osteoclast activity⁶²⁶³. In rodents, inhibition of IL-6 gene expression is in part responsible for estrogen's ability to inhibit osteoclast activation⁶⁴⁶⁵⁶⁶⁶⁷. These findings are further supported by the observation that IL-6 gene knockout mice are protected from cancellous bone loss associated with ovariectomy. IL-6 neutralizing antibody also blocks bone resorption induced by a variety of agents including TNF⁶⁸. In addition to increasing osteoclast numbers, IL-6 has been shown to stimulate bone resorption in rat long bones⁶⁹ and fetal mouse metacarpi⁷⁰, calvaria⁷¹, and bone resorption pit assays^{72 73}. Although it is not clear that IL-6 alone is sufficient to mediate these activities⁷⁴, these data demonstrate the importance of IL-6 in enhancing osteoclastic activity thus providing a mechanism for IL-6 promoting osteoporosis. IL-1beta may induce IL-6 production in human osteoblasts (MG-63 cells) by the following sequence of steps: IL-1beta-induced COX-2 activation, prostaglandin E(2) production, and PGE receptor-1 (EP-1 receptor) signaling prior to IL-6 production⁷⁵. IL-6 functions in a wide variety of other systems including the reproductive system by participating in the menstrual cycle⁷⁶ and spermatogenesis, skin proliferation, megakaryocytopoiesis, macrophage differentiation, and neural cell differentiation and proliferation⁷⁷. A significant amount of InterLeukin-6 is produced in the rat spinal cord following peripheral nerve injury that results in pain behaviors suggestive of neuropathic pain. These spinal IL-6 levels correlated directly with the mechanical allodynia intensity following nerve injury⁷⁸. During times of stress or inflammation IL-6 levels are increased. Inflammatory joint disease, particularly rheumatoid arthritis⁷⁹, is associated with increased synovial fluid levels of IL-6⁸⁰.

Interleukin-8

This is a pain-generating inflammatory mediator. In one study of patients with post herpetic neuralgia, the patients who received methylprednisolone, had interleukin-8 concentrations decrease by 50 percent, and this decrease correlated with the duration of neuralgia and with the extent of global pain relief⁸¹ (P<0.001 for both comparisons).

Interleukin-10

This is one of the natural anti-inflammatory cytokines, which also include Interleuken-1 receptor antagonist (IL-1ra), Interleukin-4, Interleukin-13 and transforming growth factor-beta1 (TGF-beta1). Interleukin-10 (IL-10) is made by immune cells called macrophages during the shut-off stage of the immune response. Interleukin-10 is a potent anti-inflammatory agent, which acts partly by decreasing the production of inflammatory cytokines interleukin-1 beta (Interleukin-1 beta), tumor necrosis factor-alpha (TNF-alpha) and inducible nitric oxide synthetase (iNOS), by injured nerves and activated white blood cells, thus decreasing the amount of spinal cord and peripheral nerve damage⁸²⁸³. In rats with spinal cord injury (SCI), a single injection of IL-10 within half an hour resulted in 49% less spinal cord tissue loss than in untreated rats. The researchers observed nerve fibers traveling straight through the spared tissue regions, across the zone of injury. They also reported a decrease in the inflammatory mediator TNF-alpha, which rises significantly after SCI.

Prostaglandins are inflammatory mediators that are released during allergic and inflammatory processes. Phospholipase A2 enzyme, which is present in cell membranes, is stimulated or activated by tissue injury or microbial products. Activation of phospholipase A2 causes the release of arachidonic acid from the cell membrane phospholipid. From here there are two reaction pathways that are catalyzed by the enzymes cyclooxygenase (COX) and lipoxygenase (LOX). These two enzyme pathways compete with one another. The cyclooxygenase enzyme pathway results in the formation of inflammatory mediator prostaglandins and thromboxane. The lipoxygenase enzyme pathway results in the formation of inflammatory mediator leukotriene. Because they are lipid soluble these mediators can easily pass out through cell membranes.

In the cyclooxygenase pathway, the prostaglandins D, E and F plus thromboxane and prostacyclin are made. Thromboxanes are made in platelets and cause constriction of vascular smooth muscle and platelet aggregation. Prostacyclins, produced by blood vessel walls, are antagonistic to thromboxanes as they inhibit platelet aggregation.

Prostaglandins have diverse actions dependent on cell type but are known to generally cause smooth muscle contraction. They are very potent but are inactivated rapidly in the systemic circulation. Leukotrienes are made in leukocytes and macrophages via the lipoxygenase pathway. They are potent constrictors of the bronchial airways. They are also important in inflammation and hypersensitivity reactions as they increase vascular permeability and attract leukocytes.

Tumor necrosis factor alpha—This inflammatory mediator is released by macrophages as well as nerve cells. Very importantly, TNF-alpha is released from injured or herniated disks. During an inflammatory response, nerve cells communicate with each other by releasing neuro-transmitter glutamate. This process follows activation of a nerve cell receptor called CXCR4 by the inflammatory mediator stromal cell-derived factor 1 (SDF-1). An extraordinary feature of the nerve cell communication is the rapid release of inflammatory mediator tumor necrosis factor-alpha (TNF alpha). Subsequent to release of TNF-alpha, there is an increase in the formation of inflammatory mediator prostaglandin. Excessive prostaglandin release results in an increased production of neurotransmitter glutamate and an increase in nerve cell communication resulting in a vicious cycle of inflammation There is excitation of pain receptors and stimulation of the specialized nerves e.g. C fibers and A-delta fibers that carry pain impulses to the spinal cord and brain.

Studies have established that herniated disk tissue (nucleus pulposus) produces a profound inflammatory reaction with release of inflammatory chemical mediators. Disk tissue applied to nerves may induce a characteristic nerve sheath injury⁸⁴⁸⁵⁸⁶ increased blood vessel permeability, and blood coagulation. The primary inflammatory mediator implicated in this nerve injury is Tumor necrosis factor-alpha but other mediators including Interleukin 1-beta may also participate in the inflammatory reaction. Recent studies have also shown that that local application of nucleus pulposus may induce pain-related behavior in rats, particularly hypersensitivity to heat and other features of a neuropathic pain syndrome.

Nitric Oxide—This inflammatory mediator is released by macrophages. Other mediators of inflammation such as reactive oxygen products and cytokines, considerably contribute to inflammation and inflammatory pain by causing an increased local production of Cyclooxygenase enzyme. The cyclooxygenase enzyme pathway results in the formation of inflammatory mediator prostaglandins and thromboxane. Concurrently to the increased production of the Cyclooxygenase-2 (COX-2) gene, there is increased production of the gene for the enzyme inducible nitric oxide synthetase (iNOS), leading to increased levels of nitric oxide (NO) in inflamed tissues. In these tissues, NO has been shown to contribute to swelling, hyperalgesia (heightened reaction to pain) and pain. NO localized in high amounts in inflamed tissues has been shown to induce pain locally and enhances central as well as peripheral stimuli. Inflammatory NO is thought to be synthesized by the inducible isoform of nitric oxide synthetase (iNOS).

Substance P (sP)—An important early event in the induction of neuropathic pain states is the release of Substance P from injured nerves which then increases local Tumor Necrosis Factor alpha (TNF-alpha) production. Substance P and TNF-alpha then attract and activate immune monocytes and macrophages, and can activate macrophages directly. Substance P effects are selective and Substance P does not stimulate production of Interleukin-1, Interleukin-3, or Interleukin-6. Substance P and the associated increased production of TNF-alpha has been shown to be critically involved in the pathogenesis of neuropathic pain states. TNF protein and message are then further increased by activated immune macrophages recruited to the injury site several days after the primary injury. TNF-alpha can evoke spontaneous electrical activity in sensory C and A-delta nerve fibers that results in low-grade pain signal input contributing to central sensitization. Inhibition of macrophage recruitment to the nerve injury site, or pharmacologic interference with TNF-alpha production has been shown to reduce both the neuropathologic and behavioral manifestations of neuropathic pain states⁸⁷

Gelatinase B or Matrix Metallo-Proteinase 9 (MMP-9)—This enzyme is one of a group of metalloproteinases (which includes collagenase and stromelysin) that are involved in connective tissue breakdown. Normal cells produce MMP-9 in an inactive, or latent form. The enzyme is activated by inflammatory mediators such as TNF-alpha and interleukin-1 that are released by cells of the immune system (mainly neutrophils but also macrophages and lymphocytes) and transformed cells⁸⁸⁸⁹. MMP-9 helps these cells migrate through the blood vessels to inflammatory sites or to metastatic sites. Activated, MMP-9 can also degrade collagen in the extra cellular matrix of articular bone and cartilage and is associated with joint inflammation and bony erosions⁹⁰. Consequently, MMP-9 plays a major role in acute and chronic inflammation, in cardiovascular and skin pathologies as well as in cancer metastasis. MMP-9 can also degrade a protein called beta amyloid. Normal cells produce MMP-9 in an inactive, or latent form, converting it to active enzyme when it is needed. But when normal brain cells producing MMP-9 fail to activate the enzyme, insoluble amyloid-b could accumulate in brain tissue. Previous research has shown that the undegraded form of amyloid-beta accumulates in the brain as senile “plaques” that signal the presence of Alzheimer's disease⁹¹.

Natural Suppression of the Inflammatory Response

How does the inflammatory response end?

Immune cells produce anti-inflammatory cytokine mediators that help to suppress the inflammatory response and suppress the production of pro-inflammatory cytokines. The natural anti-inflammatory cytokines are Interleuken-1 receptor antagonist (IL-1ra), Interleukin-10, Interleukin-4, Interleukin-13 and transforming growth factor-beta1 (TGF-beta1). Research has shown that administration of these anti-inflammatory cytokines prevents the development of painful nerve pain that is produced by a naturally occurring irritant protein called Dynorphin A⁹²

Under normal circumstances,, the inflammatory response should only last for as long as the infection or the tissue injury exists. Once the threat of infection has passed or the injury has healed, the area should return to normal existence.

One of the ways that the inflammatory response ends is by a phenomenon known as

“Apoptosis”.

Most of the time, cells of the body die by being irreparably damaged or by being deprived of nutrients. This is known as Necrotic death. However, cells can also be killed in another way, i.e. by “committing suicide”. On receipt of a certain chemical signal, most cells of the body can destroy themselves. This is known as Apoptotic death. There are two main ways in which cells can commit Apoptosis.

1. By receiving an Apoptosis signal. When a chemical signal is received that indicates that the cell should kill itself, it does so.

2. By not receiving a “stay-alive” signal. Certain cells, once they reach an activated state, are primed to kill themselves automatically within a certain period of time, i.e. to commit Apoptosis, unless instructed otherwise. However, there may be other cells that supply them with a “stay-alive” signal, which delays the Apoptosis of the cell. It is only when the primed cell stops receiving this “stay-alive” signal that it kills itself.

The immune system employs method two above. The immune cells involved in the inflammatory response, once they become activated, are primed to commit Apoptosis. Helper T cells emit the stay-alive signal, and keep emitting the signal for as long as they recognize foreign antigens or a state of injury in the body, thus prolonging the inflammatory response. It is only when the infection or injury has been eradicated, and there is no more foreign antigen that the helper T cells stop emitting the stay-alive signal, thus allowing the cells involved in the inflammatory response to die off.

If foreign antigen is not eradicated from the body or the injury has not healed, or the helper T cells do not recognize that fact, or if the immune cells receive the stay-alive signal from another source, then chronic inflammation may develop.

The final pathway for the natural suppression of the inflammatory response is in the spinal cord where there is a complex network of inhibitory neurons (gate control) that is driven by descending projections from brain stem sites. These inhibitory neurons act to dampen and counteract the spinal cord hyper excitability produced by tissue or nerve injury. Thus, peripherally evoked pain impulses pass through a filtering process involving inhibitory transmitters gamma-aminobutyric acid (GABA), glycine and enkephalins. The activity of these substances in the spinal cord usually attenuates and limits the duration of pain. In the case of persistent pain, there is evidence of pathological reduction of the supraspinal inhibitory actions in combination with ectopic afferent input in damaged nerves⁹³.

Inflammatory Pain Syndromes

Arthritis

Arthritis means inflammation of the joints. People of all ages including children and young adults can develop arthritis. The symptoms are intermittent pain, swelling, redness and stiffness in the joints. There are many different types of arthritis, some of which are rheumatoid arthritis, osteoarthritis, infectious arthritis and spondylitis. In rheumatoid arthritis, and other autoimmune diseases like systemic lupus erythematosus (SLE), the joints are destroyed by the immune system. Osteoarthritis pain is due to inflammatory stimulation of pain receptors present in bone tissue, cartilage, joints, disk, ligaments, soft tissue and muscle. A thin layer of cartilage called articular cartilage covers the bones in a synovial joint. Articular cartilage is softer than bone. The principal roles of articular cartilage are to reduce friction at the joint, to act as a cushion to absorb the shock associated with joint use and to efficiently transmit weight loads to the underlying bone. When weight is loaded onto the joint, the cartilage layer compresses. Once the weight is removed, the cartilage rebounds to its original dimensions. Articular cartilage has a milky, glass-like appearance and is composed of an extracellular matrix (ECM), which make up 98-99% of total volume and chondrocytes, which make up 1-2% of total volume⁹⁴. The ECM is made up of water, collagen (mostly type II collagen fibrils), and proteoglycans. Embedded in the ECM are the chondrocytes, the only cells of the articular cartilage. Proteoglycans are polysaccharide chain structures that have an overall negative charge due to their molecular structure. This gives them a high attraction for water. In the ECM of articular cartilage, large numbers of proteoglycans are arranged in aggregates that are tightly bound within a framework of arching collagen fibrils. The collagen fibrils form a tight network that restrains and anchors the water-loaded proteoglycans to keep them in place. Chondrocytes are widely disbursed throughout the cartilage, embedded in the ECM. Because the articular cartilage lacks blood vessels, the chondrocytes must receive nutrients and eliminate waste through the process of diffusion. Nutrients and wastes diffuse through the synovial fluid within the joint capsule and through the surrounding blood vessels. Blood vessels are located in the synovial membrane and subchondral bone⁹⁵. The integrity of the cartilage is dependent on the activity of the chondrocytes. Their function is to regulate both the synthesis and degradation of articular cartilage through the secretion of enzymes. In normal, adult articular cartilage, ECM is constantly being degraded and repaired. These two processes of degradation and repair are normally kept in balance by the activity of the chondrocytes. The chondrocytes are stimulated by and secrete a number of enzymes that help regulate the balance of synthesis and degradation of the ECM. Interleukin-1 (IL-1), a cytokine produced by chondrocytes and other cells in the joint, plays an important role in cartilage degradation by stimulating the synthesis of degradative enzymes that inhibit the production of proteoglycans. Other cytokines that appear to act synergistically with IL-1 to promote matrix breakdown are tumor necrosis factor-alpha (TNF-alpha) and interleukin-6 (IL-6). All of these cytokines are routinely found in inflamed joints. Enzymes secreted by the chondrocytes are released into the ECM and degrade the matrix structure. Among the enzymes that have been identified as playing a major role in proteoglycan and collagen degradation are the matrix metalloproteinases (MMPs), such as collagenase, stromelysin, and gelatinase. Other proteinases include cysteine, proteinases, cathepsin, and serine proteases, such as tissue plasminogen activator. Under normal circumstances, the activation of these degradative enzymes is held in check by inhibitors, such as tissue inhibitor of metalloproteinase (TIMP) and plasminogen activator inhibitor-7 (PAI-7). These inhibitors work by forming complexes that inactivate the degradative enzymes. Chondrocytes are responsible for maintaining the balance between the degradative enzymes and their inhibitors. In Osteoarthritis, there is, an imbalance between the levels of these degradative enzymes such as MMPs, and their inhibitors, such as TIMP. As part of the cartilage degradation and synthesis process, polypeptides, such as insulin-like growth factor-1 (IGF-1) and transforming growth factor beta (TGF-beta), stimulate chondrocytes in the matrix to synthesize proteoglycans. IGF-1 and TGF-beta regulate matrix metabolism in normal cartilage and may play a role in matrix repair in patients with Osteoarthritis. Excessive force being applied to the joint can activate the division and multiplication of the chondrocytes. The chondrocytes multiply and become very metabolically active. In Osteoarthritis, the biggest risk factor for activation of the chondrocytes is long term or repeated trauma as well as a previous injury to the joint, ligament or cartilage. In the early stages of Osteoarthritis, groups of chondrocytes may appear in “nests,” termed brood clusters, within the cartilage matrix. The chondrocytes produce increased quantities of proteoglycans and collagen. This may lead to an initial thickening of the articular cartilage and enable the joint to maintain normal function for years. Eventually, the arrangement and size of collagen fibers are altered and the proteoglycans begin to break down faster than they can be synthesized. The decreased proteoglycan content and altered collagen structure of the matrix result in a deterioration of the cartilage's normal physiologic properties. Early damage to the cartilage may consist of microfractures and fibrillations. As Osteoarthritis progresses, gross evidence of damage to articular cartilage becomes evident. The normally smooth surface of the cartilage becomes rough or eroded with cracks. Osteoarthritis affects not only the articular cartilage, but also the underlying bone and adjacent joint structures. As the cartilage becomes eroded, fragments may break loose and float within the joint capsule. These loose pieces of cartilage can damage the synovial lining of the joint and interfere with proper joint function. Progressive damage to the cartilage results in narrowing of the space between the two bones (joint space) because areas of bone become denuded of cartilage, causing the loss of the shock-absorbing mechanism and allowing for the contact of bone on bone⁹⁶ The underlying subchondral bone may form a new articulating surface in the joint and become smooth and polished, like marble. In subchondral bone, osteoblasts begin to form new bone tissue, probably in response to chemical messengers produced by the chondrocytes. This leads to bone remodeling. Around the edges of the joint, bony and cartilaginous overgrowths or “spurs,” called osteophytes may develop in non weight-bearing areas of the joint. Despite the loss of bone and cartilage in other areas of the joint, the presence of osteophytes tends to increase the size of osteoarthritic joints. The osteophytes frequently lead to gnarling or joint deformity. In areas where cartilage is absent, subchondral bone may appear thickened or sclerotic (“subchondral sclerosis”). In the subchondral bone, areas of focal pressure and the denuded cartilage result in necrosis of bone and bone marrow, leading to subarticular or subchondral bone cysts. Although Osteoarthritis has previously been considered a non-inflammatory form of arthritis, the underlying origin, like all other pain syndromes is inflammation and the inflammatory response (Sota Omoigui's Law of Pain). There are changes that occur within the joint that are associated with inflammation. Inflammation is aggravated by the introduction of bone and cartilage breakdown products into the synovial fluid. These products are phagocytized by cells in the synovium, resulting in chronic, low-grade inflammation. Consequently, the synovial membrane becomes thickened. Inflammation of the synovial membrane may be absent in the earlier stages of Osteoarthritis; however, as the disease progresses, some degree of synovitis usually exists.

Once mild synovial inflammation is established, the synovium becomes a source of cartilage-degrading enzymes (e.g., MMPs) and cytokines, including IL-1, IL-6, and TNF-alpha. These substances diffuse through the synovial fluid and cause further degradation of articular cartilage. IL-1 and TNF-alpha stimulate the chondrocytes to produce more degrading enzymes, and the process continues in a vicious cycle. IL-1 , IL-6, and TNF-alpha are believed to be the main cytokines linked to the disease process. Nitric oxide (NO) is found at higher levels in osteoarthritic cartilage than in normal cartilage. A form of NO can be expressed after the activation of chondrocytes by cytokines. Once formed, NO may contribute to IL-1-induced degradation of cartilage, mainly by decreasing the synthesis of the ECM. Studies have shown that IL-1 derived from the osteoarthritic cartilage stimulates the production of prostaglandin E₂ (PGE₂). Once formed, PGE₂ increases the synthesis of stromelysin, a cartilage-degrading protein (MMP). PGE₂ also has important pro-inflammatory properties and contributes to vasodilation and pain in patients with Osteoarthritis.

In rheumatoid arthritis, and other autoimmune diseases like systemic lupus erythematosus (SLE), the joints are destroyed by the immune system. TNF-alpha and Interleukin 1-beta play an important role in rheumatoid arthritis by mediating cytokines that cause inflammation and joint destruction. TNF-alpha, Interleukin 1-beta and Substance P are elevated in the joint fluids in patients with rheumatoid arthritis⁹⁷. These inflammatory mediators are also elevated in the joint fluid in patients with osteoarthritis albeit to a far less extent. Along with mechanical factors, growth factors and cytokines such as TGF beta 1, IL-1 alpha, IL-1 beta and TNF-alpha may be involved in the formation and growth of osteophytes, since these molecules can induce growth and differentiation of mesenchymal cells. The incidence and size of osteophytes may be decreased by inhibition of direct or indirect effects of these cytokines and growth factors on osteoid deposition in treated animals⁹⁸⁹⁹. Inhibition of IL-1 receptor also decreases the production of metalloproteinase enzymes collagenase-1 and stomelysin-1 in the synovial membrane and cartilage. These enzymes are involved in connective tissue breakdown¹⁰⁰.

Osteoporosis Pain

Osteoporosis is a significant health problem. As the population ages, an increasing number of men and women are developing this condition. Along with osteopenia (low bone mass), it affects nearly 25% of the population, 80% of whom are women¹¹⁰¹. It is estimated that nearly 15 million patients have osteoporosis and another 34 million patients have osteopenia¹⁰². For patients over 50 years of age, nearly half of them suffer from osteoporosis or osteopenia. Men and women lose one to three percent of their bone mass each year after age 50. Osteoporosis is a bone disease in which the amount of bone is decreased and the structural integrity of trabecular bone is impaired. Bone is a living tissue with two types of bone cells. There are osteoblasts, which are continuously building up bone and Osteoclasts, which are continually breaking down bone. The Basic Multicellular Unit (BMU) is a wandering team of cells that dissolves an area of the bone surface and then fills it with new bone. After microdamage to the bone, following mechanical stress, following exposure to some cytokines, or at random, a BMU will originate. The lining cells become active and change from a pancake-like to a cuboidal shape. Lining cells that have been activated by IL-1, PTH, calcitriol, etc (but not IL-6) will then secrete the protein receptor activator of nuclear factor-kappa B ligand (RANKL), which is required to induce osteoclastogenesis. RANK-ligand remains bound to the cell surface. Osteoblast precursors also secrete RANK-ligand. Pre-osteoclasts have membrane receptors called RANK. When RANK-ligand activates these receptors the cells fuse and differentiate into mature multinucleared osteoclasts which develop a ruffled border and resorb bone. The mature osteoclasts resorb bone. As the BMU wanders, new osteoclasts are continuously activated and then start resorption. At any one spot on the surface the resorption lasts about two weeks. The osteoclasts then undergo programmed cell death or apoptosis, which is delayed by estrogen deficiency. Osteoblasts are derived from marrow stromal cells, which can differentiate into either adipocytes or osteoblasts; the transcription factor Runx2/Cbfa1 is necessary for osteoblastic differentiation. Osteoblasts are attracted by bone-derived growth factors. The active, secreting osteoblasts then make layers of osteoid and slowly refill the cavity. They also secrete growth factors, osteopontin, osteocalcin, and other proteins. When the osteoid is about 6 microns thick, it begins to mineralize with calcium and phosphate. For months after the cavity has been filled with bone, the crystals of mineral are packed more closely and the density of the new bone increases. The final osteoblasts turn into lining cells, which participate in the minute-to-minute release of calcium from the bones. Some of the osteoblasts also turn into osteocytes, which remain in the bone, connected by long cell processes which can sense mechanical stresses to the bones. This process, also, is regulated by the osteoblasts. A balance of these bone cells maintains bone density and bone calcium. Excessive activity of osteoclasts leads to a break down of bone and leakage of calcium out of the bone into the blood. This causes a loss of bone density. There are two different kinds of bone: cortical and cancellous (trabecular) bone. Cortical bone is compact and found mostly in long bones as a shell, whereas cancellous bone consists of a network of bone trabeculae and is found mostly in the vertebrae and pelvis. Cancellous bone is more metabolic active with a larger surface area and is, therefore, more susceptible to bone resorption¹⁰³. Bone strength depends on both the density (quantity) of the bone and on the quality of the bone. Bone quality is determined by bone mass (as measured by bone density) and also by the micro-architecture of bone, the crystal size and shape, the brittleness, the connectivity of the trabecular network, the vitality of the bone cells, ability to repair micro-cracks, and the structure of the bone proteins. The fat cells, vasculature, neuronal pathways and bone marrow cells probably also influence the quality of the bone as well as the quantity of bone¹⁰⁴. In addition to bone porosity, the bone strength is determined by the trabecular microstructure. Perforations of individual trabecula occur when resorption cavities are too deep. The remaining trabecula are not as well connected and are mechanically weaker. Bone mineral density increases when more mineral is packed within the bone, even if there has been no new bone formation (in fact, this may occur because there is so little bone formation). In osteoporosis, cortical bone becomes more porous and thinner. This makes the bone weaker and more likely to fracture with the slightest trauma. Osteopenia is an early stage of Osteoporosis. Using standardized bone density measurements of the total hip, “normal” bone is greater than 833 mg/cm². “Osteopenia” is between 833 and 648 mg/cm². Osteoporosis is lower than 648 mg/cm², and “Severe (established) osteoporosis” is when there has been a pathological fracture. The World Health Organization has based bone density definitions on the T-score, which is the number of standard deviations from the mean (average) value of a 25-year-old woman. In a Normal bone, T-score is better than −1. In Osteopenia, T-score is between −1 and −2.5. In Osteoporosis, T-score is less than −2.5. Established osteoporosis includes the presence of a non-traumatic fracture and is associated with bone inflammation and bone pain. In the USA, about 21% of postmenopausal women have osteoporosis (low bone density), and about 16% have had a fracture. In women older than 80, about 40% have experienced a fracture of the hip, vertebra, arm, or pelvis¹⁰⁵¹⁰⁶. The vast majority of hip fractures occur after a fall. About 5% appear to be “spontaneous” fractures, in which the patient feels a fracture and then falls. Hip fractures are a major cause of loss of independence in older women and men. Many are discharged into nursing homes instead of back to their previous living situation. The one-year mortality following a hip fracture is 12 to 24%. Vertebral compression fractures vary in degree from mild wedges to complete compression. The symptoms also vary but are often associated with pain. In one study in 7223 older women, lateral spine radiographs were obtained at baseline and at a follow-up examination an average of 3.7 years later. New vertebral fractures, even those not recognized clinically, were associated with substantial increases in back pain and functional limitation due to back pain¹⁰⁷. Osteoporosis may be caused by endocrine diseases and abnormalities in metabolism of calcium such as hypogonadism, hyperparathyroidism, hyperthyroidism, Cushing syndrome, acidosis, Gaucher's disease. Secondary causes of Osteoporosis include renal or hepatic failure, marrow diseases including multiple myeloma, mastocytosis, thalassemia, anemia, acidosis, hypercalciuria¹⁰⁸, medications including corticosteroids, dilantin, gonadotropin releasing hormone agonists, loop diuretics, methotrexate, thyroid, heparin, cyclosporin, depot-medroxyprogesterone acetate.

Glucocorticoid-induced osteoporosis occurs as a result of decreased osteoblast-mediated bone formation, secondary increase of osteoclast-mediated bone resorption, effects on calcium homeostasis, effects on sex hormone as well as other effects¹⁰⁹. Glucocorticoid-induced decrease in bone formation is due to increased apoptosis of osteocytes and osteoblasts¹¹⁰. Long-term exposure to glucocorticoids inhibits osteoblast proliferation, attachment of osteoblasts to matrix, and synthesis of both type I collagen and noncollagenous proteins by osteoblasts¹¹¹¹¹¹². This is reflected in a dose-related reduction in the circulating levels of osteocalcin, and is likely mediated by effects on the expression of oncogenes¹¹³, prostaglandin E production¹¹⁴, and synthesis of insulin-like growth factors¹¹⁵ and transforming growth factor¹¹⁶. Decrease in collagen expression and an increase in collagenase expression leads to the degradation of type I collagen.¹¹⁷ Glucocorticoids cause a decrease in intestinal absorption of both calcium and phosphate. The mechanisms of this action are poorly understood, but are thought to be mediated by factors independent of vitamin D¹¹⁸¹¹⁹. Urinary calcium excretion is increased in glucocorticoid-treated patients, possibly due to a direct effect on tubular reabsorption of calcium¹²⁰¹²¹. Decreased gastrointestinal absorption and increased renal excretion of calcium can lead to secondary hyperparathyroidism with elevated serum levels of parathyroid hormone (PTH)¹²²¹²³. Persistently elevated PTH levels can increase bone resorption. Of note, no consistent abnormalities in vitamin D, PTH, or calcitonin levels have been found in glucocorticoid-treated patients¹²⁴¹²⁵. Glucocorticoids cause a reduction in sex hormone production both indirectly, by reducing endogenous pituitary hormone levels and adrenal androgen production, and directly, through effects on gonadal hormone release¹²⁶¹²⁷¹²⁸. Secretion of luteinizing hormone from the pituitary is decreased, with a resultant decrease in estrogen and testosterone production by the ovaries and testes, respectively. Circulating levels of estradiol, estrone, dehydroepiandrosterone sulfate, androstenedione, and progesterone are lowered in both men and women. Deficiencies of these anabolic hormones likely play a significant role in the pathogenesis of glucocorticoid-induced osteoporosis. Glucocorticoid-induced myopathy and muscle weakness may also contribute to bone loss by removing the normal forces on bone that are produced by muscle contraction. In opposition to these effects, glucocorticoids inhibit IL-6 expression. During times of stress or inflammation IL-6 levels are increased. IL-6, in turn, can induce release of corticotrophin-releasing factor¹²⁹¹³⁰, which results in elevated systemic levels of corticosteroids. These findings along with the observations that natural and synthetic corticosteroids inhibit IL-6 production from a variety of tissues^{131 132 133 134}, provide a mechanism for a negative-feedback loop. However, the overall result of the multiple effects of steroids on bone formation and resorption is a substantial loss of trabecular bone (up to 30% in some studies) in the first few months of steroid treatment¹³⁵. Most individuals using glucocorticoid drugs in doses greater than the equivalent of prednisone 5 mg/day will experience bone loss. Cross-sectional studies of patients taking these drugs long-term, demonstrate bone densities 10-20% below normal, though the distribution remains unimodal suggesting that most individuals lose bone to a similar extent. Thus, an individual's bone density while taking steroids is substantially determined by their pre-treatment density, which in turn will reflect their age, sex, body weight and underlying disease. Density is also influenced by the cumulative glucocorticoid dose. The loss of bone results in fractures in about one third of patients after 5-10 years of glucocorticoid treatment. Fractures are predominantly at sites rich in trabecular bone, such as the vertebral bodies and ribs, though hip fracture risk is also increased three-fold by these drugs. Patients receiving organ transplantations are particularly at risk, since the other immunosuppressive agents also contribute to the loss of bone.

Hereditary skeletal diseases that manifest with osteoporosis include osteogenesis imperfecta, rickets, hypophosphatasia.

Interleukin-6 is the primary chemical mediator involved in bone inflammation and bone pain. Interleukin-6 increases the activity of the osteoclasts and leads to excessive breakdown of bone, leakage of calcium into the blood, loss of bone density and bone inflammation, which is associated with bone pain. Inteleukin-6 production is increased by Inteleukin-1 beta and Tumor necrosis Factor alpha. Patients with rheumatoid arthritis(RA) develop both generalized and periarticular osteoporosis. Both of them are believed to be associated with increased production of inflammatory cytokines (TNF alpha, IL-1 beta, IL-6) and increased formation and activation of osteoclasts¹³⁶. In their youth, women are protected from osteoporosis because of the presence of sufficient levels of estrogen. Estrogen blocks the osteoblast's synthesis of Interleukin 6. Estrogen may also antagonize the interleukin 6 receptors. Transgenic mice without interleukin 6 do not develop osteoporosis after surgical removal of the ovaries (oophorectomy)¹³⁷. Estrogen's ability to repress IL-6 expression was first recognized in human endometrial stromal cells¹³⁸. Additional clues came from the observations that menopause or ovariectomy resulted in increased IL-6 serum levels¹³⁹, increased IL-6 mRNA levels in bone cells¹⁴⁰, and increased IL-6 secretion by mononuclear cells^{141 142 143}. Further evidence for estrogen's ability to repress IL-6 expression is derived from studies which demonstrated that estradiol inhibits bone marrow stromal cell and osteoblastic cell IL-6 protein and mRNA production in vitro¹⁴⁴ 140) and that estradiol was as effective as neutralizing antibody to IL-6 to suppress osteoclast development in murine bone cell cultures¹⁴⁵ or in ovariectomized mice¹⁴⁶. Osteoclast apoptosis also appears to be regulated by estrogens. With estrogen deficiency, the osteoclasts live longer and are therefore able to breakdown (resorb) more bone. In response to the increased bone resorption, there is increased bone formation and a high-turnover state develops which leads to bone loss. Within the first five years of menopause, substantial disruption of trabecular architecture can be seen. This has been demonstrated in human biopsies using micro-CT techniques. Estrogens retard the bone resorbing effects of Parathyroid Hormone (PTH). Women who are given PTH injections develop hypercalcemia and increased bone resorption; when the women are treated with estrogen before the PTH injections the effect is muted. This effect could potentially be related to effects on IL-6. Adhesion of multiple myeloma cells to stromal cells triggers IL-6 secretion by stromal cells¹⁴⁷, which may be involved in increased bone resorption in multiple myeloma. Adhesion of activated T cells induces marked production of bone-resorbing cytokines such as IL-1 and IL-6 by osteoblasts.

Paget's disease was first described by Paget in 1877 as a focal enlargement and deformity of bone. Paget originally thought the disease was a chronic inflammatory condition of bone and termed it osteitis deformans¹⁴⁸ but it is now recognized as a disease of the osteoclast—the primary bone-resorbing cell. Paget's disease is the most exaggerated example of disordered bone remodeling. Abnormal osteoclasts demonstrate increased bone resorption; followed by an abundance of new bone formation¹⁴⁹. The newly formed bone is poorly organized and structurally unsound. Only about 10% of patients with Paget's disease are symptomatic¹⁵⁰ The most common symptom, pain, is usually not due to pagetic involvement of the bone, but to osteoarthritis resulting from hypertrophy of pagetic bone at the subchondral areas of the joint¹⁵¹. Osteoclasts in pagetic lesions express high levels of interleukin-6 and interleukin-6 receptor mRNA; serum and marrow samples from patients with Paget's disease contain increased levels of interleukin-6¹⁵².

The gram positive bacterium, Staphylococcus aureus, is the major causative agent of the bone disease osteomyelitis. This disease is an often severe infection that is characterized by progressive inflammatory destruction of bone¹⁵³. Bone-forming osteoblasts are induced to secrete a number of important immune mediators when exposed to S. aureus. In one study, there was evidence for the production of significant quantities of an array of inflammatory cytokines¹⁵⁴, colony-stimulating factors,¹⁵⁵and chemokines by osteoblasts during bacterial infection of bone. The study demonstrated the expression of the key inflammatory cytokine interleukin-6 by osteoblasts in organ cultures of neonatal mouse calvaria, in a mouse model that closely resembled the pathology of trauma-induced staphylococcal osteomyelitis, as determined by confocal microscopic analysis¹⁵⁶. The study concluded that bacterial challenge of osteoblasts during bone diseases, such as osteomyelitis, induces cells to produce inflammatory molecules that can direct appropriate host responses or contribute to progressive inflammatory damage and result in progressive bone destruction. In a study of patients with chronic osteomyelitis (principally caused by Staphylococcus aureus), in plasma, and after lipopolysaccharide stimulation of whole-blood leucocytes, elevated concentrations of pro-inflammatory cytokines Tumor necrosis factor-alpha (TNF), interleukin (IL)-6 and IL-8 were detected in bone compared to plasma (all P<0.0002)¹⁵⁷.

Multiple myeloma is the malignant proliferation of plasma cells involving more than 10 percent of the bone marrow. The multiple myeloma cell produces monoclonal immunoglobulins that may be identified on serum or urine protein electrophoresis¹⁵⁸. Two thirds of patients complain of bone pain, frequently located in the back, long bones, skull and pelvis. Bone pain is related to multiple osteolytic lesions. Skeletal lesions are observed on simple radiographs in 80 percent of cases¹⁵⁹. The most common finding is diffuse osteopenia. The radiographic findings often relate to vertebral compression fractures. Like other organs with a blood supply, the bones react to the disturbances in permeability caused by inflammatory mediators. There is fluid accumulation in the bones, which is visible on Magnetic Resonance images¹⁶⁰. In addition to pain, patients may complain of nonspecific constitutional symptoms related to hyperviscosity and hypercalcemia. There may be a chronic, unresolving or incapacitating infection, commonly respiratory. Paresthesia and sensory loss may indicate neurologic damage caused by hyperviscosity, spinal cord compression or amyloid deposition. The multiple myeloma cell establishes itself in the bone marrow by adhering to stromal cells and inhibiting osteoblastic activity and osteocalcin production. Adhesion of the multiple myeloma cell stimulates production of interleukin-6 (IL-6), a paracrine and autocrine growth factor for the multiple myeloma cell. IL-6 is one of several osteoclast-activating factors produced as a result of the multiple myeloma cell-stromal cell interaction. Increased osteoclastic activity plus inhibited osteoblastic activity results in osteoporosis, painful osteolytic lesions and hypercalcemial¹⁶¹. The multiple myeloma cells also secrete IL1 β and other osteoclast activating factors (OAFs).

Our research combined with our clinical experience shows that the primary origin of osteoporosis is inflammation and the inflammatory response¹⁶². Osteoporosis has been hitherto classified into Primary Osteoporosis and Secondary Osteoporosis¹⁶³. Primary osteoporosis has been defined as a deterioration of bone mass that is unassociated with other chronic illness and is related to aging and decreased gonadal function. Early menopause or premenopausal estrogen deficiency states have been included in this classification of primary osteoporosis. Secondary osteoporosis results from chronic conditions that contribute significantly to accelerated bone loss. These chronic conditions include endogenous and exogenous thyroxine excess, hyperparathyroidism, malignancies, gastrointestinal diseases, medications, renal failure and connective tissue diseases.¹⁶⁴ On the basis of our research, this classification of Osteoporosis is outdated and needs to be revised. We have made the following classification based on physiological and pathological etiology: Inflammatory Osteoporosis and Non-Inflammatory Osteoporosis. Inflammatory Osteoporosis is due to increased bone resorption secondary to increased activity of the inflammatory mediators Interleukin 6, Interleukin 1 Beta and Tumor Necrosis Factor alpha in conditions as diverse as aging, osteoarthritis, estrogen deficiency, multiple myeloma, and infection. Non-inflammatory Osteoporosis will encompass pathological states resulting in poor bone formation such as liver disease, nutritional deficiencies and medications e.g. glucocorticoids.

Stress

During times of stress or inflammation Substance P, IL-1 and IL-6 levels are increased. IL-6, in turn, can induce release of corticotrophin-releasing factor¹⁶⁵¹⁶⁶, which results in elevated systemic levels of corticosteroids.

Back and Neck Pain

Back and neck pain most commonly results from injury to the muscle, disk, nerve, ligament or facet joints with subsequent inflammation and spasm. Degeneration of the disks or joints produces the same symptoms and occurs subsequent to aging, previous injury or excessive mechanical stresses that this region is subjected to because of its proximity to the sacrum in the lower back.

Herniated disk tissue (nucleus pulposus) produces a profound inflammatory reaction with release of inflammatory chemical mediators most especially Tumor Necrosis Factor Alpha. Subsequent to release of TNF-alpha, there is an increase in the formation of inflammatory mediator prostaglandin and Nitric Oxide. It is now known that Tumor Necrosis Factor Alpha is released by herniated disk tissue (nucleus pulposus), and is primarily responsible for the nerve injury and behavioral manifestations of experimental sciatica associated with herniated lumbar discs¹⁶⁷. This has been confirmed by numerous animal studies and research wherein application of disk tissue (nucleus pulposus) to a nerve results in nerve fiber injury, with reduction of nerve conduction velocity, intracapillary thrombus formation, and the intraneural edema formation¹⁶⁸¹⁶⁹. One study demonstrated that disk tissue (nucleus pulposus) increases inducible nitric oxide synthetase activity in spinal nerve roots and that nitric oxide synthetase inhibition reduces nucleus pulposus-induced swelling and prevents reduction of nerve-conduction velocity. According to the authors, the results suggest that nitric oxide is involved in the pathophysiological effects of disk tissue (nucleus pulposus) in disc herniation¹⁷⁰. Tumor Necrosis Factor Alpha and other inflammatory mediators induce phospholipase A2 activation. High levels of phospholipase A2 previously have been demonstrated in a small number of patients undergoing lumbar disc surgery. Phospholipase A2 is the enzyme responsible for the liberation of arachidonic acid from cell membranes at the site of inflammation and is considered to be the limiting agent in the production of inflammatory mediator prostaglandins and leukotrienes¹⁷¹. Subsequent to the release of inflammatory mediators, activation of motor nerves that travel from the spinal cord to the muscles results in excessive muscle tension, spasm and pain. The vast majority of herniated disk pain is inflammatory in origin, can be treated medically and does not require surgery. Surgery is only indicated when there is compression of the nerve roots producing significant muscle weakness and or urinary or bowel incontinence.

Fibromyalgia

Fibromyalgia is a chronic, painful musculoskeletal disorder characterized by widespread pain, pressure hyperalgesia, morning stiffness, sleep disturbances including restless leg syndrome, mood disturbances, and fatigue. Other syndromes commonly associated with fibromyalgia include irritable bowel syndrome, interstitial cystitis, migraine headaches, temporomandibular joint dysfunction, dysequilibrium including nerve mediated hypotension, sicca syndrome, and growth hormone deficiency. Fibromyalgia is accompanied by activation of the inflammatory response system, without immune activation¹⁷². In fact, there is some evidence that fibromyalgia is accompanied by some signs of immunosuppression¹⁷³. Several studies have shown that there are increased levels of the inflammatory transmitter Substance P (SP) and calcitonin gene related peptide (CGRP) in the spinal fluid of patients with fibromyalgia syndrome (FMS)¹⁷⁴¹⁷⁵¹⁷⁶. The levels of platelet serotonin are also abnormal¹⁷⁷. Furthermore, in patients with fibromyalgia, the level of pain intensity is related to the spinal fluid level of arginine, which is a precursor to the inflammatory mediator nitric oxide (NO)¹⁷⁸. Another study found increases over time in blood levels of cytokines Interleukin-6, Interleukin-8 and Interleukin-1R antibody (IL-1Ra) whose release is stimulated by substance P. The study authors concluded that because Interleukin-8 promotes sympathetic pain and Interleukin-6 induces hypersensitivity to pain, fatigue and depression, both cytokines play a role in producing FM symptoms¹⁷⁹.

Interstitial Cystitis

Interstitial cystitis is a severe debilitating bladder disease characterized by unrelenting pelvic pain and urinary frequency. This sterile painful bladder disorder is associated with a defective glycosaminoglycan bladder mucosal layer and an increased number of activated mast cells. Mast cells are ubiquitous cells derived from the bone marrow and are responsible for allergic reactions as they release numerous vasodilatory, nociceptive and pro-inflammatory mediators in response to immunoglobulin E (IgE) and specific antigen. Mast cell secretion is also triggered by a number of peptides, such as bradykinin and substance P, and may also be involved in the development of inflammatory responses¹⁸⁰. SP-containing nerve fibres are increased in the submucosa of the urinary bladder of interstitial cystitis (IC) patients and are frequently seen in juxtaposition to Mast cells¹⁸¹¹⁸². There is enhanced sympathetic innervation of the bladder in the submucosa and detrusor muscle. In interstitial cystitis the number of neurons positive for inflammatory mediator vasoactive intestinal polypeptide and neuropeptide Y is higher¹⁸³. Substance P (SP) and bradykinin (BK) influence the excitatory motor innervation of the urinary bladder. These peptides potentiate the responses to the purinergic component of the neurogenic stimulation (that part of the contractile response that remains after treatment with atropine) and potentiate the responses to exogenously applied adenosine triphosphate (ATP)¹⁸⁴. Significant elevations in Interleuken-2, Interleukin-6, and Interleukin-8 have also been found in the urine of subjects with active interstitial cystitis compared with subjects with interstitial cystitis in remission and control subjects¹⁸⁵

Migraine

Migraine headache is caused by activation of trigeminal sensory fibers by known and unknown migraine triggers. There is subsequent release of inflammatory mediators from the trigeminal nerve. This leads to distention of the large meningeal blood vessels in the skull and brain and the development of a central sensitization within the trigeminal nucleus caudalis (TNC). Genetic abnormalities may be responsible for altering the response threshold to migraine specific trigger factors in the brain of a migraineur compared to a normal individual¹⁸⁶.

The painful neurogenic vasodilation of meningeal blood vessels is a key component of the inflammatory process during migraine headache. The cerebral circulation is supplied with two vasodilator systems: the parasympathetic system storing vasoactive intestinal peptide, peptide histidine isoleucine, acetylcholine and in a subpopulation of nerves neuropeptide Y, and the sensory system, mainly originating in the trigeminal ganglion, storing inflammatory mediator substance P, neurokinin A and calcitonin gene-related peptide (CGRP)¹⁸⁷. A clear association between migraine and the release of inflammatory mediator calcitonin gene-related peptide (CGRP) and substance P (SP) has been demonstrated. Jugular plasma levels of the potent vasodilator, calcitonin gene-related peptide (CGRP) have been shown to be elevated in migraine headache. CGRP-mediated neurogenic dural vasodilation is blocked by anti-migraine drug dihydroergotamine, triptans, and opioids¹⁸⁸. In cluster headache and in chronic paroxysmal hemicrania, there is additional release of inflammatory mediator vasoactive intestinal peptide (VIP) in association with facial symptoms (nasal congestion, runny nose)¹⁸⁹. Immunocytochemical studies have revealed that cerebral blood vessels are invested with nerve fibers containing inflammatory mediator neuropeptide Y (NPY), vasoactive intestinal peptide (VIP), peptide histidine isoleucine (PHI), substance P (SP), neurokinin A (NKA), and calcitonin gene-related peptide (CGRP). In addition, there are studies reporting the occurrence of putative neurotransmitters such as cholecystokinin, dynorphin B, galanin, gastrin releasing peptide, vasopressin, neurotensin, and somatostatin. The nerves occur as a longitudinally oriented network around large cerebral arteries. There is often a richer supply of nerve fibers around arteries than veins. The origin of these nerve fibers has been studied by retrograde tracing and denervation experiments. These techniques, in combination with immunocytochemistry, have revealed a rather extensive innervation pattern. Several ganglia, such as the superior cervical ganglion, the sphenopalatine ganglion, the otic ganglion, and small local ganglia at the base of the skull, contribute to the innervation. Sensory fibers seem to derive from the trigeminal ganglion, the jugular-nodose ganglionic complex, and from dorsal root ganglia at the cervical spine level C2. The noradrenergic and most of the NPY fibers derive from the superior cervical ganglion. A minor population of the NPY-containing fibers contains vasoactive intestinal peptide (VIP), instead of NA and emanates from the sphenopalatine ganglion. The cholinergic and the vasoactive intestinal peptide (VIP)-containing fibers derive from the sphenopalatine ganglion, the otic ganglion, and from small local ganglia at the base of the skull. Most of the substance P (SP-), neurokinin A (NKA), and calcitonin gene-related peptide (CGRP)-containing fibers derive from the trigeminal ganglion. Minor contributions may emanate from the jugular-nodose ganglionic complex and from the spinal dorsal root ganglia. Neuropeptide Y (NPY), is a potent vasoconstrictor in vitro and in situ. Vasoactive intestinal peptide (VIP), peptide histidine isoleucine (PHI), substance P (SP), neurokinin A (NKA), and calcitonin gene-related peptide (CGRP) act via different mechanisms to induce cerebrovascular dilatation¹⁹⁰. Meningeal blood vessels are involved in the generation of migraine pain and other headaches. Classical experiments have shown that blood vessels of the cranial dura mater are the most pain-sensitive intracranial structures. Dural blood vessels are supplied by trigeminal nerve fibers, and dilate in response to activation of the trigeminal nerves and release of neuropeptide cytokines such as substance P (SP) and calcitonin gene-related peptide (CGRP)¹⁹¹. CGRP can be released experimentally from dural nerve fibers, and there is evidence that this occurs also during migraine attacks. Stimulation of dural nerve fibers causes vasodilatation and an increase in dural arterial flow, which depends on the release of CGRP but not SP. SP, on the other hand, is known to mediate plasma leakage (extravasation) from small veins in the dura mater. The dural arterial flow depends also on the formation of cell wall nitric oxide. The introduction of serotonin (5-HT₁) receptor agonists such as sumatriptan changed the treatment strategies for migraine. Sumatriptan and other triptans may inhibit the release of inflammatory mediators from the trigeminal nerve. Sumatriptan has been shown to block the release of vasoactive cytokines from trigeminal nerves that surround the blood vessels in the dura mater during migraine. Sumatriptan blocks nerve fiber induced plasma extravasation but has only minor effects on nerve fiber mediated vasodilatation and dural arterial flow. Foods like cheese, beer, and wine can also induce migraine in some people because they contain the mediator histamine and/or mediator-like compounds that cause blood vessels to expand. Women tend to react to histamine-containing foods more frequently than men do, on account of a deficiency in an enzyme (diamine oxidase) that breaks histamine down. Taking supplemental B6 has been shown to be helpful in migraine, as it can increase diamine oxidase activity.

NERVE (NEUROPATHIC) PAIN SYNDROMES (e.g. carpal tunnel syndrome, trigeminal neuralgia, post herpetic neuralgia, phantom limb pain)

Nociceptive pain is mediated by receptors on A-delta and C nerve fibers, which are located in skin, bone, connective tissue, muscle and viscera. These receptors serve a biologically useful role at localizing noxious chemical, thermal and mechanical stimuli. Nociceptive pain can be somatic or visceral in nature. Somatic pain tends to be well-localized, constant pain that is described as sharp, aching, throbbing, or gnawing. Visceral pain, on the other hand, tends to be vague in distribution, spasmodic in nature and is usually described as deep, aching, squeezing and colicky in nature. Examples of nociceptive pain include: post-operative pain, pain associated with trauma, and the chronic pain of arthritis.

Neuropathic pain, in contrast to nociceptive pain, is described as “burning”, “electric”, “tingling”, and “shooting” in nature. It can be continuous or paroxysmal in presentation. Whereas nociceptive pain is caused by the stimulation of peripheral A-delta and C-polymodal pain receptors, by inflammatory mediators, (e.g. histamine bradykinin, substance P, etc.) neuropathic pain is produced by injury or damage to peripheral nerves or the central nervous system

The hallmarks of neuropathic pain are chronic allodynia and hyperalgesia. Allodynia is defined as pain resulting from a stimulus that ordinarily does not elicit a painful response (e.g. light touch). Hyperalgesia is defined as an increased sensitivity to normally painful stimuli.

Examples of neuropathic pain include carpal tunnel syndrome, trigeminal neuralgia, post herpetic neuralgia, phantom limb pain, complex regional pain syndromes and the various peripheral neuropathies. Subsequent to nerve injury, there is increase in nerve traffic. Expression of sodium channels is altered significantly in response to injury thus leading to abnormal excitability in the sensory neurons. Nerve impulses arriving in the spinal cord stimulate the release of inflammatory protein Substance P. The presence of Substance P and other inflammatory proteins such as calcitonin gene-related peptide (CGRP) neurokinin A, vasoactive intestinal peptide removes magnesium induced inhibition and enables excitatory Inflammatory proteins such as glutamate and aspartate to activate specialized spinal cord NMDA receptors. This results in magnification of all nerve traffic and pain stimuli that arrive in the spinal cord from the periphery. In one study, monocytes/macrophages (ED-1), natural killer cells, T lymphocytes, and the pro-inflammatory cytokines tumor necrosis factor-alpha (TNF-alpha) and interleukin-6 (IL-6), were significantly produced in nerve-injured rats. Interestingly, ED-1-, TNF-alpha- and InterLeukin-6-positive cells increased more markedly in allodynic rats than in non-allodynic ones. The magnitude of the inflammatory response was not related to the extent of damage to the nerve fibers because rats with complete transection of the nerves displayed much lower production of inflammatory cytokines than rats with partial transection of the nerve¹⁹². This is a finding commonly observed in patients where a minor injury results in severe pain that is out of proportion to the injury. Getting back to the study, the authors determined that the considerable increase in monocytes/macrophages induced by a nerve injury results in a very high release of Interleukin-6 and TNF-alpha. This may relate to the generation of touch allodynia/hyperalgesia, since there was a clear correlation between the number of ED-1 and Interleukin-6-positive cells and the degree of allodynia. Abnormal development of sensory-sympathetic connections follow nerve injury, and contribute to the hyperalgesia (abnormally severe pain) and allodynia (pain due to normally innocuous stimuli). These abnormal connections between sympathetic and sensory neurons arise in part due to sprouting of sympathetic axons. Studies have shown that sympathetic axons invade spinal cord dorsal root ganglia (DRG) following nerve injury, and activity in the resulting pericellular axonal ‘baskets’ may underlie painful sympathetic-sensory coupling¹⁹³. Sympathetic sprouting into the DRG may be stimulated by neurotrophins such as nerve growth factor (NGF), brain derived neurotrophic factor (BDNF), neurotrophin-3 (NT-3) and neurotrophin 4/5 (NT-4/5). In another study, animals exhibiting heat hyperalgesia as a sign of neuropathic pain seven days after loose ligation of the sciatic nerve exhibited a significant increase in the concentration of brain derived neurotrophic factor (BDNF) in their lumbar spinal dorsal horn.¹⁹⁴ Administration of nerve growth factor to rodents has resulted in the rapid onset of hyperalgesia. In clinical trials with nerve growth factor for the treatment of Alzheimer disease and peripheral neuropathy, induction of pain has been the major adverse event¹⁹⁵. In one study, the use of trkA-IgG, an inhibitor of Nerve Growth Factor (NGF) reduced neuroma formation and neuropathic pain in rats with peripheral nerve injury¹⁹⁶ In another study, the systemic administration of anti-nerve growth factor (NGF) antibodies significantly reduced the severity of autotomy (self mutilating behavior induced by nerve damage) and prevented the spread of collateral sprouting from the saphenous nerve into the sciatic innervation territory¹⁹⁷. Activity in sympathetic fibers is associated with excessive sweating, temperature instability of the extremities and can induce further activity in sensitized pain receptors and, therefore, enhance pain and allodynia (sympathetically maintained pain). This pathologic interaction acts via noradrenaline released from sympathetic terminals and newly expressed receptors on the afferent neuron membrane¹⁹⁸.

Activation of motor nerves that travel from the spinal cord to the muscles results in excessive muscle tension. More inflammatory mediators are released which then excite additional pain receptors in muscles, tendons and joints generating more nerve traffic and increased muscle spasm. Persistent abnormal spinal reflex transmission due to local injury or even inappropriate postural habits may then result in a vicious circle between muscle hypertension and pain¹⁹⁹. Separately, constant C-fiber nerve stimulation to transmission pathways in the spinal cord results in even more release of inflammatory mediators but this time within the spinal cord. The transcription factor, nuclear factor-kappa B (NF-kappaB), plays a pivotal role in regulating the production of inflammatory cytokines²⁰⁰. Inflammation causes increased production of the enzyme cyclooxygenase-2 (Cox-2), leading to the release of chemical mediators both in the area of injury and in the spinal cord. Widespread induction of Cox-2 expression in spinal cord neurons and in other regions of the central nervous system elevates inflammatory mediator prostaglandin E₂ (PGE₂) levels in the cerebrospinal fluid. The major inducer of central Cox-2 upregulation is inflammatory mediator interleukin-1^{β} in the CNS²⁰¹. Basal levels of the enzyme phospholipase A₂ activity in the CNS do not change with peripheral inflammation. The central nervous system response to pain can keep increasing even though the painful stimulus from the injured tissue remains steady. This “wind-up” phenomenon in deep dorsal neurons can dramatically increase the injured person's sensitivity to the pain.

The neurotrophins are a family of growth promoting proteins that are essential for the generation and survival of nerve cells during development, Neurotrophins promote growth of small sensory neurons and stimulate the regeneration of damaged nerve fibers They consist of four members, nerve growth factor (NGF), brain derived neurotrophic factor (BDNF), neurotrophin-3 (NT-3) and neurotrophin 4/5 (NT-4/5).

Nerve growth factor and brain-derived neurotrophic factor modulate the activity of a sodium channel (NaN) that is preferentially expressed in pain signaling neurons that innervate the body (spinal cord dorsal root ganglion neurons) and face (trigeminal neurons). Transection of a nerve fiber (axotomy) results in an increased production of inflammatory cytokines and induces marked changes in the expression of sodium channels within the sensory neurons²⁰². Following axotomy the density of slow (tetrodotoxin-resistant) sodium currents decrease and a rapidly repriming sodium current appears. The altered expression of sodium channels leads to abnormal excitability in the sensory neurons²⁰³. Studies have shown that these changes in sodium channel expression following axotomy may be attributed at least in part to the loss of retrogradely transported nerve growth factor²⁰⁴.

In addition to effects on sodium channels, there is a large reduction in potassium current subtypes following nerve transection and neuroma formation. Studies have shown that direct application of nerve growth factor to the injured nerve can prevent these changes²⁰⁵.

Reflex Sympathetic Dystrophy/Chronic Regional Pain Syndrome (RSD/CRPS)

Reflex sympathetic dystrophy (RSD) syndrome also called Chronic Regional Pain Syndrome (CRPS) has been recognized clinically for many years. It is most often initiated by trauma to a nerve, neural plexus, or soft tissue. Diagnostic criteria are the presence of regional pain and other sensory changes following a painful injury. The pain is associated with changes in skin color, skin temperature, abnormal sweating, tissue swelling. With time, tissue atrophy may occur as well as involuntary movements, muscle spasms, or pseudoparalysis²⁰⁶. Like other organs with a blood supply, the bones also react to the disturbances in permeability caused by various inflammatory mediators. There is fluid accumulation in the bones and loss of bone density (osteoporosis)²⁰⁷. In addition, the inflammatory mediators accelerate the rate at which bone is broken down. The bone loss is further aggravated by decreased use of the affected body part due to pain. Complex regional pain syndrome, type I (reflex sympathetic dystrophy; CRPS-I/RSD) can spread from the initial site of presentation. In one study of 27 CRPS-I/RSD patients who experienced a significant spread of pain, three patterns of spread were identified. ‘Contiguous spread (CS)’ was noted in all 27 cases and was characterized by a gradual and significant enlargement of the area affected initially. ‘Independent spread (IS)’ was noted in 19 patients (70%) and was characterized by the appearance of CRPS-I in a location that was distant and non-contiguous with the initial site (e.g. CRPS-I/RSD appearing first in a foot, then in a hand). ‘Mirror-image spread (MS)’ was noted in four patients (15%) and was characterized by the appearance of symptoms on the opposite side in an area that closely matched in size and location the site of initial presentation. Only five patients (19%) suffered from CS alone; 70% also had IS, 11% also had MS, and one patient had all three kinds of spread²⁰⁸. In 1942 Paul Sudeck suggested that the signs and symptoms of RSD/CRPS including sympathetic hyperactivity might be provoked by an exaggerated inflammatory response to injury or operation of an extremity. His theory found no followers, as most doctors incorrectly believe that RSD/CRPS is solely initiated by a hyperactive sympathetic system. Recent research and studies including various clinical and experimental investigations now provide support to the theory of Paul Sudeck²⁰⁹.

As we now understand, soft tissue or nerve injury causes excitation of sensory nerve fibers. Reverse (antidromic) firing of these sensory nerves causes release of the inflammatory neuropeptides at the peripheral endings of these fibers. These neuropeptides may induce vasodilation, increase vascular permeability, attract other immune cells such as T helper cells and excite surrounding sensory nerve fibers—a phenomenon referred to as neurogenic inflammation. At the level of the central nervous system, the increased input from peripheral pain receptors alters the central processing mechanisms.

Sympathetic dysfunction, which often has been purported to play a pivotal role in RSD/CRPS, has been suggested to consist of an increased rate of outgoing (efferent) sympathetic nerve impulses towards the involved extremity induced by increased firing of the sensory nerves. However, the results of several experimental studies suggest that sympathetic dysfunction also consists of super sensitivity to catecholamines induced by nerve injury (autonomic denervation)²¹⁰. Part of this occurs due to injured sensory nerves and immune cells developing receptors for the chemical transmitter norepinephrine and epinephrine (catecholamines), which are normally released by sympathetic nerves and also circulate in the blood. Stimulation of these receptors by locally released or circulating catecholamines produces sympathetic effects such as sweating, excessive hair growth and narrowing of blood vessels²¹¹. In addition and under certain conditions, catecholamines may boost regional immune responses, through increased release of Interleukin-1, tumor necrosis factor-alpha, and Interleukin-8 production.

In several studies, patients with RSD/CRPS showed a markedly increased level of the inflammatory peptide bradykinin as well as calcitonin gene-related peptide²¹². The levels of bradykinin were four times as high as the controls. A few showed increased levels of the other inflammatory chemical mediators²¹³. Two pain producing pathways have been identified: inflammatory stimuli induce the production of bradykinin, which stimulates the release of TNF-alpha . The TNF-alpha induces production of (i) Interleukin-6 and Interleukin-1b, which stimulate the production of cyclooxygenase products, and (ii) Interleuken-8, which stimulates production of sympathomimetics (sympathetic hyperalgesia)²¹⁴.

Abnormal development of sensory-sympathetic connections follow nerve injury, and contribute to the hyperalgesia (abnormally severe pain) and allodynia (pain due to normally innocuous stimuli). These abnormal connections between sympathetic and sensory neurons arise in part due to sprouting of sympathetic axons. This can be induced by neurotrophins such as nerve growth factor (NGF), brain derived neurotrophic factor (BDNF), neurotrophin-3 (NT-3) and neurotrophin 4/5 (NT-4/5).

Sports Injuries/Bursitis/Tendonitis/Rotator Cuff Tears

Inflammation of the bursa is known as bursitis. A bursa is a small sac containing fluid that lies between bone and other moving structures such as muscles, skin or tendons. The bursa allows smooth gliding between these structures. A bursa allows a tendon or muscle to move smoothly over a bone by acting as an anti-friction device and shielding the structures from rubbing against bones. Bursae are found in the knee, elbow, shoulder and wrist. If the tendons become thickened and bumpy from excessive use, the bursa is subjected to increased friction and may become inflamed. Tendonitis is inflammation or irritation of a tendon. Tendons are the thick fibrous cords that attach muscles to bone. They function to transmit the power generated by a muscle contraction to move a bone. Since both tendons and bursae are located near joints, inflammation in these soft tissues will often be perceived by patients as joint pain and mistaken for arthritis. Symptoms of bursitis and tendonitis are similar: pain and stiffness aggravated by movement. Pain may be prominent at night. Almost any tendon or bursa in the body can be affected, but those located around a joint are affected most often. The most common cause of tendonitis and bursitis is injury or overuse during work or play, particularly if the patient is poorly conditioned, has bad posture, or uses the affected limb in an awkward position. Occasionally an infection within the bursa or tendon sheath will be responsible for the inflammation. Tendonitis or bursitis may be associated with diseases such as rheumatoid arthritis, gout, psoriatic arthritis, thyroid disease and diabetes. In one study of thirty-nine patients with rotator cuff diseases, the levels of the cytokine Interleukin-1 beta was significantly correlated with the degree of pain. The combined results of immunohistochemistry indicated that both synovial lining and sublining cells produce IL-1beta, while synovial lining cells predominantly produce the anti-inflammatory intracellular InterLeukin-1 receptor antagonist (icIL-1ra) and sublining cells secrete secreted InterLeukin-1 receptor antagonist (sIL-1ra)²¹⁵. In another study, the levels of interleukin-1 beta were significantly higher in the shoulder joints in patients with anterior instability and chronic inflammation of the joint²¹⁶. In another study, immunohistological staining demonstrated the expression of Interleukin-1 beta (Interleukin-1 beta), Tumor necrosis factor alpha (TNF-alpha), transforming growth factor beta (TGF-beta), and basic fibroblast growth factor (bFGF) in subacromial bursa derived from the patients suffering from rotator cuff tear²¹⁷.

Vulvar Vestibulitis Syndrome (VVS)/Vulvodynia

Vulvar vestibulitis syndrome is a major subtype of vulvodynia. It is a constellation of symptoms and findings involving and limited to the vulvar vestibule that consists of: (1) severe pain on vestibular touch to attempted vaginal entry, (2) tenderness to pressure localized within the vulvar vestibule, and (3) physical findings confined to vulvar erythema of various degrees. The syndrome has been seen in association with subclinical human papillomavirus, chronic recurrent candidiasis, chronic recurrent bacterial vaginosis, chronic alteration of vaginal pH, and the use of chemical and destructive therapeutic agents²¹⁸. In a study of VVS cases and asymptomatic controls, median tissue levels of inflammatory cytokines: IL-1 b and TNF-a, from selected regions of the vulva,, vestibule, and vagina were 2.3-fold and 1.8-fold elevated, respectively, in women with VVS compared to pain-free women. Analysis revealed a significant 2.2-fold higher median level of TNF alpha at the vulvar site compared to the vestibule. Cytokine elevations correlated poorly with inflammatory cell infiltrate and suggested cytokine production from another cell source. The study authors concluded that inflammatory cytokine elevation may contribute to the pathophysiology of mucocutaneous hyperalgesia²¹⁹

Inflammatory Mediator Blocker Medications

Vasoactive Intestinal Peptide Blocker Medications

Botulinum Toxins (Botox, Myobloc)

Botulinum toxins are potent nerve toxins, which bind to transport proteins in nerve cells and block the release of nerve transmitters from nerve endings. One of these transmitters called acetylcholine is released by nerve cells and transported into muscle cells to signal the muscle to contract. Blockade of this transmitter by Botulinum toxin can produce a long lasting relief of muscle spasms. By interfering with transport proteins in nerve cells, studies have shown that Botulinum toxin may also inhibit the release of excitatory nerve transmitter glutamate²²⁰ and inflammatory mediators such as Arachidonic acid (AA)²²¹, vasoactive intestinal peptide (VIP) and Neuropeptide Y (NPY)²²². Botulinum toxins also inhibits the release of tumor necrosis factor alpha²²³ (TNF-alpha) from immune cells and thus can alleviate pain and spasm produced by the inflammatory response.

Tumor Necrosis Factor Alpha Blocker Medications

The central role in inflammatory responses have InterLeukin-1 and TNF-alpha because the administration of their antagonists, such as IL-1ra (Interleukin-1 receptor antagonist), soluble fragment of Interleukin-1 receptor, or monoclonal antibodies to TNF-alpha and soluble TNF receptor, all block various acute and chronic responses in animal models of inflammatory diseases.

Etanercept (Enbrel)

Etanercept is a fusion protein produced by recombinant DNA technology. Etanercept binds specifically to both tumor necrosis factor alpha (TNF-alpha) and tumor necrosis factor beta (TNF-beta), and blocks their interaction with cell surface TNF receptors Etanercept binds to and inactivates Tumor Necrosis Factor (TNF-alpha) but does not affect TNF-alpha production or serum levels. Etanercept may also modulate other biologic responses that are induced or regulated by TNF-alpha such as production of adhesion molecules, other inflammatory cytokines and matrix metalloproteinase-3 (MMP-3 or stromelysin). Patients with rheumatoid arthritis have increased levels of TNF-alpha in their joint fluid. The introduction of Etanercept transformed the treatment of rheumatoid arthritis. Etanercept decreases the inflammation and inhibits the progression of structural damage in patients with moderately to severely active rheumatoid arthritis. When Etanercept was added in patients who had persistent disease despite receiving Methotrexate, rapid and sustained improvement was noted. Etanercept has been used successfully in the treatment of other inflammatory disorders. In one study, TNF-alpha blockade with Etanercept was markedly effective in controlling the clinical manifestations of inflammatory back pain located in the cervical spine, lumbar spine and sacro-iliac joints²²⁴. In another study, Etanercept was found to reduce pain and hyperalgesia in an animal model of painful neuropathy. Treatment with Etanercept by local near-nerve injection to the injured nerve or by systemic application significantly reduced thermal hyperalgesia and mechanical hypersensitivity to pain. The effect of Etanercept was present in animals that were treated from the time of surgery and in those that were treated from day 6, when hypersensitivity to pain was already present. The authors conclude that the results suggest the potential of Etanercept as a treatment option for patients with neuropathic pain²²⁵. In another research study, two tumor necrosis factor-alpha inhibitors (Etanercept and Infliximab) prevented the reduction of nerve conduction velocity and nerve fiber injury produced by application of disk tissue (nucleus pulposus) to a nerve²²⁶.

Infliximab (Remicade)

Infliximab is a monoclonal antibody targeted against tumor necrosis factor-alpha (TNF-alpha). Infliximab neutralizes the biological activity of the cytokine tumor necrosis factor-alpha (TNF-alpha). Infliximab binds to high affinity soluble and transmembrane forms of TNF-alpha and inhibits the binding of TNF-alpha with its receptors. Infliximab does not neutralize TNF-beta, a related cytokine that utilizes the same receptors as TNF-alpha. Biological activities attributed to TNF-alpha include induction of pro-inflammatory cytokines such as interleukin (IL)-1 and IL-6; enhancement of leukocyte migration by increasing endothelial layer permeability; expression of adhesion molecules by endothelial cells and leukocytes; activation of neutrophil and eosinophil functional activity; fibroblast proliferation; synthesis of prostaglandins; and induction of acute phase and other liver proteins. In patients with rheumatoid arthritis, infliximab substantially improves clinical symptoms when given in combination with Methotrexate. In patients with rheumatoid arthritis, infliximab treatment reduces inflammatory cell infiltration into inflamed areas of the joint and reduces the expression of molecules mediating adhesion [E-selectin, intercellular adhesion molecule-1 (ICAM-1), and vacular adhesion molecule-1 (VCAM-1)], chemoattraction (monocyte chemotactic protein (MCP-1 and IL-8), and tissue degradation (matrix metalloproteinase (MMP) 1 and 3). In patients with Crohn's disease, infliximab reduces infiltration of inflammatory cells and TNF-alpha production in inflamed areas of the intestine. In addition, the proportion of mononuclear cells from the lamina propria able to express TNF-alpha and interferon gamma is reduced. After treatment with infliximab, patients with Crohn's disease or rheumatoid arthritis have decreased concentrations of IL-6 and C-reactive protein as compared to baseline. Interleukin-1 Beta Blocker Medication

Anakinra (Kineret)

Anakinra is a form of the human interleukin-1 receptor antagonist (IL-1Ra) produced by recombinant DNA technology. Anakinra differs from the naturally occurring native human IL-1Ra in that it has an additional methionine residue at its amino terminus. Anakinra acts similarly to the naturally occurring interleukin-1 receptor antagonist (IL-1Ra). IL-1Ra blocks effects of Interleukin-1 by competitively inhibiting binding of this cytokine, specifically IL-alpha and IL-beta, to the interleukin-1 type 1 receptor (IL-1R1), which is produced in a wide variety of tissues. Il-1Ra is part of the feedback loop that is designed to balance the effects of inflammatory cytokines. During clinical trials, rheumatoid arthritis patients treated with Anakinra experienced clinical responses, including improvement in swollen and painful joints within 4 weeks, and most by 13 weeks, of therapy. After 6 months of therapy, 38% of patients treated with Anakinra, alone or in combination with Methotrexate, achieved a 20% improvement in the American College of Rheumatology criteria.

Interleukin-6 Blocker Medication

Leflunomide (Arava)

Leflunomide interferes with RNA and protein synthesis in immune T and B-lymphocytes. T and B cell collaborative actions are interrupted and antibody production is suppressed. Leflunomide is the first agent for rheumatoid arthritis that is indicated for both symptomatic improvement and retardation of structural joint damage. Leflunomide may also have anti-inflammatory properties secondary to reduction of histamine release, and inhibition of induction of cyclooxygenase-2 enzyme (COX-2). Leflunomide may decrease proliferation, aggregation and adhesion of peripheral and joint fluid mononuclear cells. Decrease in the activity of immune lymphocytes leads to reduced cytokine and antibody-mediated destruction of joints and attenuation of the inflammatory process.

Interleukin-6 Blocker Medication

Bisphosphonate Bone Medications

Alendronate, Clodbronate, Pamidronate (Fosamax, Aredia)

Bisphosphonates originally were used to soften hard water. This class of drugs reduces bone turnover and bone loss. The primary origin of bone loss and osteoporosis is inflammation. Interleukin-6 (IL-6) and its soluble receptor (sIL-6R) stimulate osteoclast formation and activity and accelerate the rate at which bone is broken down, resulting in bone pain and a loss of bone density, which can progress to osteoporosis. The bone loss may be further aggravated by decreased use of the affected body part due to pain. In patients with Paget's disease of bone, bisphosphonate therapy is associated with a significant reduction of Interleukin-6 soluble receptor (sIL-6R) serum levels²²⁷. Bisphosphonates inhibit the production of pro-inflammatory cytokine interleukin-6 in tumoral cell lines of human osteoblastic phenotype (MG63 and SaOs cells), and in peripheral blood mononuclear cells (PBMC)²²⁸. Bisphosphonates also inhibit IL-1 and TNF-alpha stimulated IL-6 release in cultures of human osteoblastic osteosarcoma cells²²⁹. Osteoblasts exposed to small amounts of bisphosphonate elaborate a soluble inhibitor, which interferes with osteoclast formation and development²³⁰. Bisphosphonates prevent apoptosis of murine osteocytic MLO-Y4 cells, whether it is induced by etoposide, TNF-alpha, or glucocorticoid dexamethasone²³¹. Pamidronate and other bisphosphonates inhibit the production by osteoblasts of the inflammatory cytokine interleukin-6, a growth factor essential to myeloma cells²³². In prostate cancer, bisphosphonates inhibit tumor induced osteoclastic bone resorption, thereby preventing skeletal related events and treatment induced bone loss. Bisphosphonates may also modify the bone microenvironment so that it becomes less favorable for the growth and survival of metastases²³³. Solid cancers metastasize to bone by a multi-step process that involves interactions between tumor cells and normal host cells. Some tumors, most notably breast and prostate carcinomas, grow avidly in bone because the bone microenvironment provides a favorable soil. In the case of breast carcinoma, the final step in bone metastasis (namely bone destruction) is mediated by osteoclasts that are stimulated by tumor cell production of interleukin-6 and the tumor peptide parathyroid hormone-related peptide (PTH-rP). Production of PTH-rP by breast carcinoma cells in bone is enhanced by growth factors produced as a consequence of normal bone remodeling, particularly activated transforming growth factor-beta (TGF-beta). Thus, a vicious cycle exists in bone between production by the tumor cells of mediators such as PTH-rP and subsequent production by bone of growth factors such as TGF-beta, which enhance PTH-rP production. The metastatic process can be interrupted either by neutralization of PTH-rP or by rendering the tumor cells unresponsive to TGF-beta. Bisphosphonates inhibit the effects of growth factors in bone matrix (IGFs, FGF-2) on MCF-7 and T47D cell proliferation and inhibit their protective effects on apoptotic cell death in vitro under serum-free conditions. This could happen through an interaction with growth factors' intracellular phosphorylation transduction pathways, such as ERK1/2-MAPK²³⁴. Another key factor in tumor-induced promotion of osteoclast activity is the protein receptor activator of nuclear factor-kappa B ligand (RANKL), which is required to induce osteoclastogenesis. RANKL is produced by prostate cancer bone metastases, enabling these metastases to induce osteolysis through osteoclast activation. Osteoprotegerin, is a soluble decoy receptor for RANKL and inhibits RANKL-induced osteoclastogenesis. Osteoprotegerin has been shown in murine models to inhibit tumor-induced osteolysis. Finally, matrix metalloproteinases (MMPs) are secreted by prostate cancer cells and promote osteolysis primarily through degradation of the nonmineralized bone matrix. Interleukin-1 (IL-1) and IL-6 with sIL-6R enhance the messenger RNA (mRNA) expression of MMP-13 (collagenase 3), MMP-2 (gelatinase A), MMP-9 (gelatinase B), and MMP-3 (stromelysin 1), which are associated with increases in bone matrix degradation²³⁵. MMP inhibitors have been shown to diminish tumor establishment in bone in murine models. Thus, many factors derived from prostate and breast cancer metastases can promote osteolysis, and these factors may serve as therapeutic targets²³⁶. Bisphosphonate drugs can inhibit osteoclasts and osteolysis and as a consequence of this inhibition, there is a marked reduction in the skeletal events associated with metastatic cancer to bone, such as pain, fracture, and hypercalcemia. However and possibly even more importantly, there is also a reduction of tumor burden in bone. In experimental situations, this has clearly been shown to affect not only morbidity but also survival.

Bisphosphonate induction of macrophage apoptosis is associated with the inhibitory effect on TNF-alpha release²³⁷. In one study of osteoprotegerin (OPG) and pamidronate, on joint inflammation and bone destruction using a tumor necrosis factor alpha (TNF alpha)-transgenic mouse model, a significant reduction of osteoclast numbers was seen in animals treated with OPG alone or in combination with pamidronate as well as in animals treated with infliximab. The authors concluded that OPG alone or in combination with bisphosphonates is an effective therapeutic tool for the prevention of TNF alpha-mediated destruction of bone by reducing the number of bone-resorbing cells in the inflammatory tissue²³⁸. Another study investigated the effect of oral alendronate (AL) therapy on bone turnover and osteoclast activating factors in early Rheumatoid Arthritis. A significant decrease of IL-1, IL-6, TNF-alpha, and beta2m was observed after 30 days, persisting after 90 days in the AL group, but with no significant variation in the placebo group²³⁹. In a randomized placebo-controlled multicentre trial in multiple myeloma, when compared with placebo, pamidronate treatment was associated with a marked decreased osteoclastic resorption rate, significant reduction of circulating soluble interleukin-6 receptor (IL-6sR) and a uniform tendency of lower serum and marrow plasma levels of IL-6, IL-1beta, and TNFalpha²⁴⁰. The mechanism by which bisphosphonates inhibit osteoclasts thus represents a combination of inhibition of osteoclast formation as well as increased apoptosis in mature osteoclasts. In one study, bisphosphonates (risedronate, pamidronate, and clodronate) caused a 4- to 24-fold increase in the proportion of osteoclasts showing the characteristic morphology of apoptosis in vitro²⁴¹. Studies with potent bisphosphonates such as ibandronate, pamidronate, and risedronate have clearly documented that reduction of bone turnover and osteoclast activity leads to beneficial effects not only on skeletal complications associated with metastatic cancer, but also on tumor burden in bone²⁴². Bisphosphonates are used in the treatment of osteolytic bone lesions of multiple myeloma, bone pain due to Paget's disease, postmenopausal osteoporosis, bone metastasis in patients with advanced cancer and in the treatment of elevated calcium levels associated with cancer. In one study, the efficacy and the safety of Pamidronate was assessed in patients in various stages of recalcitrant reflex sympathetic dystrophy (RSD/CRPS). Some patients had more than one site involved. Mean duration of the disease was 15 months. About half of the patients have been previously treated unsuccessfully by sympathetic blockades. Pamidronate was administered intravenously for 1-3 consecutive days. Efficacy was assessed by a decrease of pain. A significant decrease of pain was observed. These results suggest an efficacy of Pamidronate in recalcitrant RSD²⁴³. The most common adverse event typically associated with bisphosphonate therapy is transient fever. This is an acute-phase reaction that is associated with transient increase in the serum levels of TNF-alpha and IL-6²⁴⁴.

In conclusion, Bisphosphonates inhibit production of Interleukin-6 (IL-6) and its soluble receptor (sIL-6R), and thus inhibit osteoclast formation and activity and decrease the rate at which bone is broken down. This results in a decrease in inflammatory bone and joint pain. In multiple myeloma and some tumors, most notably breast and prostate carcinomas, which grow avidly in bone, bisphosphonates not only reduce bone complications and related pain, thereby improving quality of life, but also may have intrinsic anti-tumor activity by virtue of inducing tumor cell adherence to marrow, reducing interleukin-6 secretion, inducing tumor cell apoptosis, or inhibiting angiogenesis²⁴⁵.

Phosphodiesterase Inhibitor Medication

Pentoxifylline

Pentoxifylline is a phosphodiesterase inhibitor, which is used as a blood thinner medication in persons who have poor peripheral circulation. However the drug has another unique effect. It suppresses inflammatory cytokine production by T cells and macrophages²⁴⁶. Some of the anti-inflammatory effects occur by blocking nitric oxide (NO) production by macrophages. Pentoxifylline also blocks the production of Tumor Necrosis Factor Alpha. In one study, Pentoxifylline prevented nerve root injury and swelling (dorsal root ganglion compartment syndrome) caused by topical application of disk tissue (nucleus pulposus)²⁴⁷

Antibiotic Medication

Clarithromycin (Biaxin)

Studies have shown that injured joint cells produce cytokine inflammatory mediators including IL-1beta, IL-6, IL-8, granulocyte colony-stimulating factor (G-CSF) and granulocyte-macrophage colony-stimulating factor (GM-CSF. Clarithromycin significantly inhibits the production of these cytokines and also suppresses the proliferation of immune T cells²⁴⁸.

Tetracyclines (Doxycycline, Minocycline)

Tetracyclines such as doxycycline and minocycline may block a number of cytokines including Interleukin-1^{249 250}, IFNg²⁵¹, NO-synthetases, and metalloproteinases²⁵². Interleukin-1 and IFN-.gamma act synergistically with TNF-alpha and are known to be toxic to nerve tissue^{253 254 255 256 257}. One study showed that oral administration of doxycycline prevented the breakdown of cartilage in subjects with osteoarthritis²⁵⁸. In another study, a patient with rheumatoid arthritis who did not respond to other arthritis medications had marked improvement with Minocycline²⁵⁹. In another study, minocycline-treated patients were more likely to have gone in remission and discontinued treatment with prednisone at 2 years than patients who were treated with other standard rheumatoid arthritis medications²⁶⁰. Tetracyclines may also block the inflammatory cytokine Tumor Necrosis Factor Alpha (TNF-alpha). Tumor Necrosis Factor Alpha is released by herniated disk tissue (nucleus pulposus), and is primarily responsible for the nerve injury and behavioral manifestations of experimental sciatica associated with herniated lumbar discs²⁶¹ In one study, treatment with doxycycline significantly blocked the nucleus-pulposus-induced reduction of conduction velocity²⁶²

Anti-Nausea Serotonin (5-HT3) Blockers

Ondansetron (Zofran)

In migraine, 5-HT3-receptor antagonists show moderate efficacy, as well. Repeatedly demonstrated efficacy of 5-HT3-receptor antagonists such as Tropisetron in patients suffering from fibromyalgia raises the question for the mechanism of action involved. Ligand binding at the 5-HT3-receptor causes manifold effects on other neurotransmitter and neuropeptide systems. In particular, 5-HT3-receptor antagonists diminish serotonin-induced release of substance P from C-fibers and prevent unmasking of NK2-receptors in the presence of serotonin. These observations possibly provide an approach for the causal explanation of favorable treatment results with 5-HT3-receptor antagonists in fibromyalgia²⁶³.

Free Radical Scavenger Medications

DMSO (Dimethyl Sulfoxide)

A scavenger of oxygen radicals, topical DMSO inhibits nerve conduction and decreases inflammatory swelling. DMSO local anti-inflammatory effects provide symptomatic relief when the solution is applied in the bladder (intra-vesically) in patients with interstitial cystitis. A crossover study was performed for patients with RSD/CRPS to evaluate the therapeutic efficacy of the hydroxyl radical scavenger DMSO. All patients were given DMSO locally 5 times a day during one week, and a placebo during one week. Before and after each treatment, both the patient and the examiner performed subjective evaluation of the clinical activity of RSD/CRPS. Measurement was then performed of the range of motion (ROM) of all joints in the affected extremity. DMSO was the most effective treatment as to improvement of ROM (p=0.035) and as to overall improvement (p=0.001). The authors concluded that the efficacy of the hydroxyl radical scavenger DMSO indicates that RSD/CRPS primarily involves an inflammatory process rather than a sympathetic reflex. The authors further stated that during the last 20 years no single report was published studying RSD in terms of inflammation. The authors then suggested that such studies are urgently needed to elucidate the real nature of RSD/CRPS²⁶⁴

Anti-Depressant Medications

Protriptyline (Vivactil)

2000 years ago, St John's wort, a herbal anti-depressant was used to treat sciatic and nerve pain. Studies have shown that it is only the older tricyclic class anti-depressants like protriptyline or desipramine that are effective in the treatment of persistent pain. Newer SSRI class anti-depressants like Prozac and Paxil are not effective. The analgesic effects of Protriptyline and other cyclic type antidepressants may occur partly through the alleviation of depression, which may be responsible for increased pain suffering, but also by mechanisms that are independent of mood effects. Current research suggests that the pain-relieving effect of antidepressants is due to their blockade of reuptake of chemical transmitters norepinephrine and serotonin. The resulting increase in the levels of these chemical transmitters enhances the activation of pain inhibiting pathways that descend from the brain to the spinal cord. Activation of these pathways decreases the transmission of pain impulses from injured or inflamed nerves to the spinal cord dorsal horn wherein the impulses are transmitted to the brain. Amitriptyline and other cyclic antidepressants may also enhance the analgesic effect of opioid medication by increasing their efficacy of binding to opioid receptors. Protriptyline (and other cyclic antidepressants) may have a blocking effect on spinal N-methyl-D-aspartate (NMDA) receptors, and inhibit NMDA receptor activation-induced neuroplasticity²⁶⁵. Spinal NMDA receptor activation is believed to be central to the generation and maintenance of persistent hyperalgesic pain. Anti-depressant medication may also have effects on inflammatory mediators. In one study, four weeks of prolonged administration of amitriptyline and desipramine resulted in a significant increase in the secretion of the anti-inflammatory cytokine Interleukin-10²⁶⁶.

Anti-Seizure Medications

Oxcarbazepine (Trileptal)

Subsequent to tissue injury, the expression of sodium channels in nerve fibers is altered significantly thus leading to abnormal excitability in the sensory neurons. Studies have shown that the inflammatory mediators interleukin-1beta, interleukin-6, interleukin-1 receptor antagonist and inducible nitric oxide synthetase are significantly increased when there is excessive nerve traffic as occurs during seizures or persistent pain²⁶⁷. Anti-seizure medications such as Trileptal or Zonegran decrease pain by reducing the rate of continuing discharge of injured and inflamed nerve fibers. Blockade of sodium channels in nerve cells leads to a decrease in electrical activity and a subsequent reduction in release of the excitatory nerve transmitter glutamate. Anti-seizure drugs also inhibit the initiation and propagation of painful nerve impulses by inhibiting Nitric Oxide Synthetase activity²⁶⁸. Nitric Oxide Synthetase is the enzyme responsible for the production of the inflammatory mediator Nitric Oxide. Anti-seizure drugs may also protect nerve cells from free radical damage by Nitric Oxide and/or hydroxyl radicals (OH*)²⁶⁹. In one study, the anti-seizure drug Sodium valproate was shown to significantly inhibit immune cell production of TNF-alpha and Interleuken-6²⁷⁰. Sodium valproate suppresses TNF-alpha and IL-6 production via inhibition of activation of the nuclear transcription factor kappa B (NF-kappaB). In immune cells and human nerve cells, NF-kappaB is essential to the expression of inflammatory cytokines.

In addition anti- seizure medications reduce painful muscle spasm. Spasticity from different causes is associated with a deficiency of inhibitory nerve transmitters like gamma aminobutyric acid or an excess of excitatory nerve transmitters such as glutamate. Anti-seizure drugs enhance the inhibition of nerve-muscle activity by gamma aminobutyric acid in the spinal cord²⁷¹.

Thalidomide and Thalidomide Analogues

Thalidomide and analogues mainly inhibit tumor necrosis factor alpha (TNF-alpha) synthesis but the drugs also have effects on other cytokines. Thalidomides increase the production of the anti-inflammatory cytokine interleukin-10 (IL-10) in lesioned sciatic nerves. In addition, Thalidomides stimulate the release of the pain relieving natural opioid peptide methionine-enkephalin in the dorsal horn of the spinal cord²⁷².

In a recent case report, a 43-year-old woman had injured her hand and developed a severe case of RSD/CRPS that confined her to bed or a wheelchair most of the time. Three years after developing RSD/CRPS, the woman was diagnosed with multiple myeloma. She was started on thalidomide, which has shown promise for treating multiple myeloma. The change in the woman's condition was “astounding,” as reported by the authors. Within a month, the woman experienced an unexpected improvement in RSD/CRPS symptoms, which nearly disappeared²⁷³

Statins

The ability of HMG-CoA reductase inhibitors to lower C-reactive protein levels has recently brought into question the mechanisms of action of the statin drugs. One study examined the effects of atorvastatin on soluble adhesion molecules, interleukin-6 (IL-6) and brachial artery endothelial-dependent flow mediated dilatation (FMD) in patients with familial (FH) and non-familial hypercholesterolemia (NFH)²⁷⁴. A total of 74 patients (27 FH and 47 NFH) were recruited. Fasting lipid profiles, soluble intercellular adhesion molecule-1 (sICAM-1), soluble vascular-cellular adhesion molecule-1 (sVCAM-1), E-selectin, IL-6 and FMD were measured at baseline, 2 weeks, 3 and 9 months post-atorvastatin treatment (FH—80 mg/day, NFH—10 mg/day). In both groups, compared to baseline, sICAM-1 levels were significantly reduced at 2 weeks, further reduced at 3 months and maintained at 9 months (P<0.0001). The IL-6 levels were significantly reduced at 3 months and 9 months compared to baseline for FH (P<0.005) and NFH (P<0.0001). In both groups, the FMD at 2 weeks was higher than baseline (P<0.005), with progressive improvement up to 9 months. FMD was negatively correlated with sICAM-1 and IL-6.

3-hydroxy-3-methylglutaryl coenzyme A reductase inhibitors (statins) have been shown to stimulate bone formation in laboratory studies, both in vitro and in vivo. Statin use in most, but not all observational studies is associated with a reduced risk of fracture, particularly hip fracture, even after adjustment for the confounding effects of age, weight and other medication use. This beneficial effect has not been observed in clinical trials designed to assess cardiovascular endpoints²⁷⁵. In one study analysis of covariance model that was adjusted for age, body mass index, race, and vitamin use, men using statin drugs were more likely to have a greater BMD of the spine (p<0.005). The mean difference (effect size) was 0.05 g/cm2 (95% confidence interval [CI] of 0.02-0.09), about 5.3% greater BMD. In women, the association was not significant. The risk of osteoporosis (defined as a T-score<or =−2.5) was determined using logistic regression analysis after adjustment for potential confounding variables. Although not statistically significant, men who received statin drugs for more than 2 yr were approximately half as likely to develop osteoporosis (odds ratio [OR]=0.55, 95% CI=0.28-1.08). A similar effect was observed in women taking statins for any length of time (OR=0.36, 95% CI=0.12-1.07). The authors of the study suggest that statin drugs may decrease osteoporosis risk, warranting a randomized controlled trial²⁷⁶. In a cross-sectional study set in southeastern Australia, the researchers evaluated the association between statin use, fracture risk, and BMD in 1375 women (573 with incident fractures and 802 without incident fracture, all drawn from the same community). Fractures were identified radiologically. Medication use and lifestyle factors were documented by questionnaire. Unadjusted odds ratio for fracture associated with statin use was 0.40 (95% confidence interval [CI], 0.23-0.71). Adjusting for BMD at the femoral neck, spine, and whole body increased the odds ratio to 0.45 (95% CI, 0.25-0.80), 0.42 (95% CI, 0.24-0.75), and 0.43 (95% CI, 0.24-0.78), respectively. Adjusting for age, weight, concurrent medications, and lifestyle factors had no substantial effect on the odds ratio for fracture. Statin use was associated with a 3% greater adjusted BMD at the femoral neck (P=0.08), and BMD tended to be greater at the spine and whole body but did not achieve statistical significance. The researchers concluded that the substantial 60% reduction in fracture risk associated with statin use is greater than would be expected from increases in BMD alone²⁷⁷. In vitro, cerivastatin inhibits parathyroid hormone (PTH)-stimulated bone resorption. Using a panel of 40 statin analogs, which showed variable effects on HMG-CoA reductase activity, a research study found that the ability of statin compounds to inhibit bone resorption is directly related to HMG-CoA reductase activity²⁷⁸. In this study, the ability of statins to inhibit bone resorption was found to be directly related to their inhibitory effect on HMG-CoA reductase activity. In the abstract of the study, the authors stated as follows: ‘Statins, which are inhibitors of 3-hydroxy-3-glutaryl-coenzyme A (HMG-CoA) reductase, decrease the hepatic biosynthesis of cholesterol by blocking the mevalonate pathway. Nitrogen-containing bisphosphonate drugs also inhibit the mevalonate pathway, preventing the production of the isoprenoids, which consequently results in the inhibition of osteoclast formation and osteoclast function. The authors hypothesized that statins could affect bone metabolism in vivo through effects on osteoclastic bone resorption. In contrast to other studies, none of the statins tested showed anabolic effects in parietal bone explant cultures. Taken together, the authors concluded that statins inhibit bone resorption in vitro, which correlates directly with the potency of the compounds for inhibition of HMG-CoA reductase activity.

Anti-Inflammatory Medications

Non-steroidal anti-inflammatories, such as aspirin, tolmetin sodium, indomethacin and ibuprofen, inhibit the enzyme cyclooxygenase and therefore decrease prostaglandin synthesis. Prostaglandins are inflammatory mediators that are released during allergic and inflammatory processes. Phospholipase A2 enzyme, which is present in cell membranes, is stimulated or activated by tissue injury or microbial products. Activation of phospholipase A2 causes the release of arachidonic acid from the cell membrane phospholipid. From here there are two reaction pathways that are catalyzed by the enzymes cyclooxygenase and lipoxygenase. The cyclooxygenase enzyme pathway results in the formation of inflammatory mediator prostaglandins and thromboxane.

New generation Non-steroidal anti-inflammatories, such as Licofelone inhibit both enzymes cyclooxygenase and lipoxygenase therefore decreasing prostaglandin and leukotriene synthesis.

Corticosteroid

Glucocorticoids are naturally occurring hormones that prevent or suppress inflammation and immune responses when administered at pharmacological doses. At the molecular level, unbound glucocorticoids readily cross cell membranes and bind with high affinity to specific cytoplasmic receptors. This binding induces a response by modifying transcription and, ultimately, protein synthesis to achieve the steroid's intended action. Such actions can include: inhibition of leukocyte infiltration at the site of inflammation, interference in the function of mediators of inflammatory response, and suppression of humoral immune responses. Some of the net effects include reduction in edema or scar tissue and a general suppression in immune response. The degree of clinical effect is normally related to the dose administered. The anti-inflammatory corticosteroids inhibit the activation of phospholipase A₂ by causing the synthesis of an inhibitory protein called lipocortin. It is lipocortin that inhibits the activity of phospholipases and therefore limits the production of potent mediators of inflammation such as prostaglandins and leukotriene.

Corticosteroids are also effective for some types of neuropathic pain and complex regional pain syndromes. One study examined the effects of systemic methylprednisolone on acute pain and pain hypersensitivity in normal and neuropathic rats. In this study, when systemic methylprednisolone was started immediately after sciatic and saphenous nerve injury, there was a dose-dependent reduction in autotomy behavior. Substance P is an inflammatory mediator of neuropathic pain and edema. Single dose methylprednisolone (12 mg/kg) slightly reduced the substance P mediated inflammation induced with electrical stimulation of the saphenous nerve. Chronic methylprednisolone (3.4 mg/kg per day for 28 days) severely reduced the neurogenic inflammation induced with saphenous nerve stimulation. Rats with sciatic nerve injury developed hind paw edema between 7 and 14 days after surgery, and this neuropathic edema did not develop in rats chronically treated with methylprednisolone (3.4 mg/kg per day). The study results demonstrate that corticosteroids did not affect pain thresholds in normal or neuropathic rats. However, chronic steroid treatment did prevent the development of autotomy and neuropathic edema, and completely blocked neurogenic extravasation, findings consistent with the hypothesis that primary afferent substance P release mediates autotomy pain behavior and neuropathic edema²⁷⁹.

Opioid Medication

Opioid medication such as Methadone, Oxycodone, Morphine, Demerol and Vicodin produce pain relief by binding and activating specialized opioid receptors at the site of tissue injury and in an area of the spinal cord called the substantia gelatinosa. Once activated, the opioid receptors inhibit the release of inflammatory mediators such as bradykinin at site of tissue injury and Substance P from pain transmitting C nerve fibers. The pain receptors that were previously excited are now suppressed. There is also suppression of the signal traffic in the specialized nerves e.g. C fibers and A-delta fibers that carry pain impulses to the spinal cord and brain. Morphine and other opioids also alter emotional processing of painful input by acting on opioid receptors in the limbic and cortical area of the brain. In addition, new research now shows that morphine and other opioids have additional anti-inflammatory effects. These effects include:

1. Inhibition of Interleukin-1 beta converting enzyme (ICE), a proteolytic enzyme that converts the inactive precursor of interleukin-1 beta (Interleukin-1 beta) to its mature active form²⁸⁰

2. Inhibit inflammatory cytokine mediators interferon-alpha IFN (IFN-alpha) and interferon-beta (IFN-beta) production by lymphocytes and fibroblast cells²⁸¹

3. Inhibits tumor necrosis factor-alpha (TNF-alpha) production by activated macrophages²⁸²

4. Induces the suicidal cell death (apoptosis) of immune cell lymphocytes.

5. Increases the release of anti-inflammatory cytokines such as transforming growth factor-beta1 (TGF-beta1) and Interleuken-10²⁸³.

Morphine and other opioids are also effective anti-migraine agents. In electrophysiological studies morphine significantly attenuated brainstem neuronal activity in response to electrical stimulation of the dura by 65%. Morphine also inhibited the trigeminal nucleus caudalis (TNC) neuronal sensitization following calcitonin gene-related peptide (CGRP)-evoked dilation. Studies have demonstrated that opioids block the nociceptive neurotransmission within the trigeminal nucleus caudalis and in addition inhibit neurogenic dural vasodilation via an action on mu-opioid receptors located on trigeminal sensory fibres innervating dural blood vessels²⁸⁴. These peripheral and central actions could account for the anti-migraine actions of opioids²⁸⁵

Nerve Blockade

The role of neural or nerve blocks with local anesthetics with or without anti-inflammatory agents in the treatment and relief of persistent pain is well defined. A nerve fiber is a long cylinder surrounded by a semi permeable (allows only some substances to pass) membrane. This membrane is made up of proteins and lipids (fats). Some of the proteins act as channels, or pores, for the passage of sodium and potassium ions through the membrane.

The conduction of nerve impulses along a nerve fiber is associated with a change in the permeability of the membrane. The channels widen, and sodium ions (Na+) move to the inside of the fiber. At the same time, potassium ions (K+) diffuse out through other channels. As these electrolytes change positions, an electrical charge is set up and the impulses will travel down the nerve fiber. This process is called depolarization. Once the nerve impulse has passed, the channels become smaller. Sodium ions (Na+) are now “pumped” out of the fiber and potassium ions (K+) are pumped back in. The nerve membrane is now repolarized and ready to conduct another impulse.

Local anesthetic agents stabilize nerve membrane by inhibiting the sodium influx required for the initiation and conduction of impulses. The local anesthetic effect of numbness lasts as long as the agent maintains a certain critical concentration in the nerve membrane.

Subsequent to tissue injury, the expression of sodium channels in nerve fibers is altered significantly thus leading to abnormal excitability in the sensory neurons. Studies have shown that the inflammatory mediators interleukin-1beta, interleukin-6, interleukin-1 receptor antagonist and inducible nitric oxide synthetase are significantly increased when there is excessive nerve traffic as occurs during seizures or persistent pain²⁸⁶. Local anesthetic agents like anti-epileptic medications decrease pain by reducing the rate of continuing discharge of injured and inflamed nerve fibers. Blockade of sodium channels in nerve cells leads to a decrease in electrical activity and a subsequent reduction in release of the excitatory nerve transmitter glutamate. Researchers have found that preemptive analgesia—delivering pain medication to patients before or just after surgery—results in significant pain reduction long afterward—for a period that significantly exceeds the duration of action of the local anesthetic or analgesic medication. Beginning pain treatment before or immediately after surgery can vastly decrease post-operative pain^{287 288 289}.

Case Reports—Osemwota Omoigui MD

Mrs. A

A 64-year old lady presenting with a 10-year history of Paget's disease, chronic low back pain and coccydynia following a fall. She has had bilateral total hip replacement and right knee replacement. MRI of the lumbar spine showed diffuse multilevel degenerative changes. She was under the care of an endocrinologist who administered pamidronate infusion to treat her hypercalcemia of Paget's disease. The patient only received pamidronate when her serum calcium was elevated. We observed that the patient's low back pain and coccydynia, which were refractory to lumbar facet injection using steroids and chemodenervation injections with botulinum toxin, would improve significantly whenever she received an infusions of pamidronate for her hypercalcemia. There was a period of time when her endocrinologist stopped giving the patient the pamidronate infusion since her serum calcium level had normalized. The patient was experiencing severe pain and we administered diphenhydramine (benadryl) 25 mg IM as premedication before pamidronate 30 mg in 1000 mls of Normal Saline infusion was given over 3 hours. Within 24 hours, she experienced significant relief with the Pamidronate infusion despite a normal serum calcium level.

Mrs. B

A 72-year-old female with a 36-year history of low back pain—post L3, L5 lumbar laminectomy. She presented with complaints of deep-seated pain in the bones of the bilateral thighs and legs. Her history also included lumbar muscle spasm, radicular pain in bilateral legs and osteoarthritis in bilateral knees. Pain in bilateral thighs and legs started gradually and progressively worsened. Pain was described as a constant ache, unrelated to the severity of her lower back pain. The pain was also different from her previous radicular pain. The patient did not report any numbness or tingling in both legs. Her presenting pain score was 7 on a numeric pain scale of 0-10. Since her laminectomy, she has been receiving Chemodenervation of lumbar peripheral nerves and paraspinal muscles with Botulinum Toxin Type A, and Lumbar Facet and Sacroiliac joint injections using Ketorolac and Dexamethasone. Her low back pain has been under good control using this treatment regimen. She also receives etanercept (Enbrel) subcutaneous injection in her lumbar region and knees intermittently for exacerbations of her pain. However, patient continued to complain of the deep-seated aching in her thighs and legs that was not relieved by her Chemodenervation procedure and Enbrel injection. She was 20-years post-menopausal and she has not been on any hormone replacement therapy. Significant positive and negative physical examination findings included an antalgic gait, moderate tenderness of the facet joints with muscle spasm and guarding of the posterior paravertebral erector spinae muscles of the spine at L4-S1, with a positive Sacroiliac Distraction Test and moderate spasm of the posterior muscles of bilateral Sacroiliac joint. Lumbar range of motion was restricted; flexion was at 80 degrees and extension at 20 degrees. The bilateral thighs were moderately tender with no redness or swelling. Homan's sign was negative in bilateral legs. The neurological examination was normal. The working diagnoses were muscle spasm, Postmenopausal Osteoporosis, Lumbar Facet Arthropathy, Sacroilitis and Osteoarthritis. Subsequently blood specimen was drawn for serum electrolytes including ionized calcium levels, and the levels were within normal ranges. The patient was given diphenhydramine 25 mg IM as premedication before pamidronate 30 mg in 1000 mls of Normal Saline infusion was given over 3.5 hours. The patient was discharged with a prescription for alendronate 70 mg PO once weekly and Calcium 500 mg/Vitamin D 200 IU (Oscal D) PO three times daily. On re-evaluation one week later, the patient stated that the deep aching pain in her thighs and her knee pain almost completely resolved within few hours of receiving pamidronate. Her pain score dropped from the pre- pamidronate treatment level of 7 to post-treatment level of 3. The bone density of the T12, L1 and L2 vertebral bodies was assessed using a spinal CT scanner and findings were compatible with a diagnosis of Osteopenia.

Mrs. C

A 47-year old female presented with a 2-week history of severe pain in the thighs and legs bilaterally. The pain was said to have started gradually without any immediately preceding trauma. The pain was a severe constant ache graded 9 on the numeric scale of 0 to 10, aggravated by activity and unrelieved by pain medications. The patient also had a 14-year history of neck and lower back pain that started following a Motor Vehicle Accident in which her car was rear-ended at a traffic stop light by another car. Initial X-rays done at the Emergency Room following the accident were normal. However, MRI done one month later for persisting neck and lower back pain showed degenerative disk disease in the cervical and lumbar spine with herniated cervical disks. The patient at presentation was 14 years post-cervical laminectomy (C2, C3, C4 and C5). She has had several cervical and lumbar facet injections and epidural injections using steroids since the onset of her neck and low back pain. All of these treatments only gave the patient temporary relief. Her lumbar and cervical pain was under better control with periodic Chemodenervation of cervical and lumbar peripheral nerves and paraspinal muscles with Botulinum Toxin Type A, along with periodic alternating injections of subcutaneous anakinra (Kineret) and subcutaneous etanercept (Enbrel). At the time of presentation, the patient's neck and lower back pain was mild at score of 4. The low back pain intermittently radiates to bilateral lower extremities with burning sensation in the legs and feet. The patient's past medical history is also significant for pulmonary embolism, and two episodes of DVT. She is currently on Coumadin 1.25-2.5 mg daily depending on her INR. The patient is post-menopausal with a partial hysterectomy done 19 years ago for symptomatic fibroids. She was initially on Hormone Replacement Therapy that was discontinued due to recurrent DVT. Significant physical examination finding were normal gait, moderate tenderness of the bilateral thigh and legs. There was no swelling or redness. Homan's sign was negative. There was also mild tenderness of the cervical-thoracic Paraspinal muscles at C3-C7 and T1-T4 levels with restricted cervical flexion (40 degrees) and extension (20 degrees). Mild tenderness of the facet joints with muscle spasm and guarding of the posterior Paravertebral erector spinae muscles of the spine at L4-S1, with a positive Sacroiliac Distraction Test and moderate spasm of the posterior muscles of bilateral Sacroiliac joint was also noted on physical examination. Lumbar range of motion testing revealed restricted flexion at 80. Neurological examination revealed decreased pinprick sensation along the C6, C7 and L4, S1 dermatomes. The diagnoses were Thoracic and Lumbar Facet Arthropathy, Post-laminectomy Cervical Syndrome, Muscle spasm, and Osteoporosis. Blood specimen was drawn for serum electrolytes, and ionized calcium. Also a CT Bone Density testing was requested. Subsequently, the patient received an infusion of pamidronate 30 mg IVPB and methylprednisolone succinate 125 mg IVP after receiving promethazine 25 mg IM as premedication. On re-evaluation one week later, the patient stated that the pain in her bilateral lower extremities resolved completely for 6 days after receiving the pamidronate infusion. The patient stated that her pain was at a score of 4 on the day of re-evaluation and that she was pain free the day before her re-evaluation. The pamidronate infusion was repeated on two other visits with similar results. However, the CT Bone Density result was normal.

Mr. D.

A 27-year old male presented with an 11-year history of low back pain following a motor vehicle accident. Injuries sustained during the accident included burst fracture of the lumber spine at the L2 and L3 levels, and a fractured pelvic bone. The patient had a history of repeated surgeries in the spine with multiple fusions, the placement of a Harrington Rod which was later removed and the use of pedicle screws. On presentation, low back pain was a severe constant aching, graded nine on a pain scale of one to ten. Pain was radiating to both lower extremities experienced as a burning, with numbness and tingling on both thighs and legs.

Physical examination revealed a scar over the lumber spine in the midline. There was marked tenderness from the thoracic spine T9 to the sacrum S1. In addition, there was moderate spasm of the lumber paraspinal muscles. Range of motion was reduced to thirty degrees of flexion at the lumber spine; extension was limited to five degrees. Sensory perception of a pinprick was significantly reduced in both right and left L2 to L4 dermatomes. However, the motor strength was normal globally.

Initial treatment consisted of a refill of Oxycodone SR 40 mg, 2-3 tabs P.O. q 12 hrs. Other medications prescribed were Roxicodone 15 mg, 1 tab P.O. q 4-6 hrs, Tizanidine 2 mg P.O. bid, 4 mg P.O. q hs, Oxcarbazepine 300 mg P.O bid and Tolmetin DS 400 mg P.O tid with meals. The patient was subsequently scheduled for a chemodenervation procedure with Botulinum toxin. Two months later chemodenervation of the lumber paraspinal muscles with Botulinum toxin was carried out. This resulted in a drop in pain score from nine to five within five days of the procedure. More relief was noted in the aching pain and spasms in the lower back following the procedure compared to the burning pain felt in the lower extremities. Two additional chemodenervation procedures were done over a period of six months. Each time after the procedures, the pain score in the lower back would drop further than before. The most dramatic pain relief was observed after Anakinra injection 100 mg was given subcutaneous; the pain score dropped from a score of ten to two in twenty minutes. The Anakinra injection was repeated three weeks later with similar result.

Ms. E

39-year old female presented with a twenty-month history of aching and burning pain on the entire left side of the body. Onset of pain was preceded by a cerebro-vascular accident resulting in paralysis and paresthesia of the left side of the body. The pain felt by the patient was constant and severe; graded ten on a scale of one to ten. There was also muscle spasms associated with her pain. The patient was diagnosed to have hypertension at the age of eighteen years, had coronary angioplasty for recurrent angina at the age of thirty-five years. Blood work done before surgery revealed a deficiency in Protein S. A family history of hypertension, Protein S deficiency and Lupus was also noted. She was on Warfarin, Atenolol, Amlodipine, Acetaminophen 300 mg/codeine 30 mg, Carisoprodol and Amitriptyline. Physical examination showed hyperesthesia and hyperpathia on the left side of the body. Also noted were increased motor tone, spasticity and hyperreflexia on the left upper and lower limbs. A working diagnosis of neuropathic pain and spastic hemi paresis, post CVA was made.

The patient was commenced on Tizanidine 2 mg P.O bid, 4 mg P.O qhs, acetaminophen 2.5 mg/Oxycodone 325 mg 1 tab P.O q 6 hr, prn pain, and Oxcarbazepine 300 mg P.O two times daily. She was told to discontinue Carisoprodol and amitriptyline that she had been taking. A two-week appointment was made for review and to receive Etanercept injection.

On re-evaluation two weeks later, her pain score had dropped to six on the pain scale. Subsequently, she was given Etanercept 25 mg injection subcutaneous in her left arm. She was re-evaluated one week later and she gave the information that her pain score dropped from six to two within six hours of receiving the Enbrel injection.

Mr. F.

45-year-old male presented with sixteen-year-old history of low back pain and four year old history of neck pain. Pain started gradually without an immediate preceding trauma. However he had several falls on his previous construction jobs. His pain was constant, severe, radiates to both upper and lower extremities with associated numbness and tingling. The radicular symptoms were felt in the left leg and toes, right and left third to fifth fingers. He also complained of muscle spasms in the lower back and thighs, and in the shoulders.

Previous MRIs done had showed multiple-level disc bulges and degenerative changes in both the cervical and lumbar spine. He has had several surgical procedures done prior to presentation. These procedures included lumbar laminectomy (L3-L5), diskectomies and nerve root blocks in the cervical and lumbar spine. All these only afforded him temporary pain relief.

Moderate tenderness was noted in the cervical spine and cervical paraspinal muscles on examination, with moderate reduction in range of motion. He also had mild tenderness in the muscles around the right and left shoulders. Moderate tenderness was also noted in the lumbosacral spine with spasms and stiffness in the lumbar paraspinal muscles. The range of motion, however, was full. Neurological examination revealed decreased sensory perception of the pinprick on both right and left C8 dermatome. A diagnosis of post laminectomy lumbar syndrome, lumbar and cervical facet arthropathy with radiculopathy was made.

Subsequently, chemodenervation of the peripheral nerves and paraspinal muscles was done using Botulinum toxin. In addition, the patient was injected with Anakinra 100 mg subcutaneously. On re-evaluation one week later, the patient gave the information that his pain dropped significantly. His back and neck pain score dropped from a value of nine to three within one hour of receiving the injections. His radicular symptoms improved one day after the procedures.

Mr. G.

43-year-old male was being treated for chronic low back pain when he complained of severe, and constant burning pain and hypersensitivity in his skin and joints throughout the entire body, worse in the extremities. No information was given at this initial presentation about any precipitating factor. There was no history of fever or malaise. Prior to presentation, he was being treated with Clarithromycin after a diagnosis of phlebitis and toxic neuropathy. He did not comply with the antibiotic treatment despite obtaining some relief. Physical examination showed the patient was in moderate distress, but alert and oriented. Body temperature and blood pressure were within normal range. Chest examination was normal. The skin was erythematous, especially overlying the veins, with generalized hyperesthesia and allodynia. He also had mild to moderate tenderness in the bilateral shoulder, elbow, wrist and knee joints without joint swelling or heat.

A diagnosis of neurogenic inflammation to rule out Rheumatoid arthritis and phlebitis was made. The patient was told to complete the course of antibiotics. And he was to continue pain medications and therapy while awaiting blood test results from work up performed. On re evaluation one week later, he was still having severe burning pain, which was made worse after bathing in a warm Jacuzzi. Blood work results were still pending. The patient then gave the information that he had injected himself with adulterated cocaine prior to the onset of his burning pain. He had gone to see a Toxicologist who analyzed the remaining sample of the drug that was injected. The injected cocaine was found to be adulterated with chlormezanone (Trancopal). Subsequently, he was placed on Leflunomide 100 mg P.O once daily for three days, then 20 mg P.O once daily and Methadone 10 mg P.O q 6 hrs. On re-evaluation one week later, his burning pain had improved tremendously with the pain score dropping to 2/10 from an initial score of 6/10.

Mr. H

45-year-old male presented with complaints of severe pain in his right shoulder after falling on the shoulder from a height of about three feet. His pain was constant and severe, with associated difficulty abducting the joint. Patient had been seen by an orthopedic surgeon who had ordered an MRI of the right shoulder. The MRI revealed a complete rotator cuff tear involving the anterior aspect of the supraspinatus tendon adjacent to the intertuberous sulcus. The patient was advised to get immediate surgical repair of his rotator cuff. When the patient presented in our clinic he was in a lot of pain. Examination revealed severe tenderness to palpation of the right rotator cuff. His range of motion examination showed a severe limitation of abduction at 20/180 degrees. Mr. C. N. was placed on Tolmetin sodium 400 mg P.O. three times daily with meal and Oxycodone 5 mg 1-2 tabs P.O. q 4 hr. He had only a slight improvement on the medications. He was subsequently given Anakinra 100 mg subcutaneously. Within two minutes of administration of the Anakinra, patient was able to fully raise his right shoulder to 180/180 degrees and was quite surprised. On re-evaluation one week later, he gave the information that his pain dropped from a score of 9/10 to 3/10 within five minutes of receiving the Anakinra injection. The duration of pain relief lasted for one month. He was given a second injection of Anakinra 100 mg SC that has given sustained pain relief for five months till the time of publication.

Mrs. I.

53-year-old female with a five-year history of generalized body pain involving the joints and soft tissue. She has been receiving specialist pain management following a diagnosis of fibromyalgia, myofascial pain and osteoarthritis. She also had a four-year history of intermittent abdominal pain, worse in the lower abdominal regions, passage of loose stool with occasional bloodstains. After undergoing endoscopy with biopsy, her abdominal condition was diagnosed to be ulcerative colitis by a Gastroenterologist. As part of her chronic pain management, she was given Anakinra 100 mg subcutaneously. This resulted in relief of her joint and soft tissue pain and remission of her ulcerative colitis as evidenced by resolution of abdominal pain within two days of the injection. This remission lasted for six months up till the time of publication. The remission was accompanied by an increase in appetite and a slight gain in weight.

Mrs. J.

40-year-old female presented with a fourteen-year history of generalized body pain. Pain was described as severe, constant aching, aggravated by activity, relieved slightly and transiently by pain medications. She also had insomnia, extreme fatigue, and a history of Irritable Bowel Disease. Her primary care physician had diagnosed her to have Fibromyalgia. She has also had several tender point injections with local anesthetic before her referral for pain management. On examination, the patient could only walk with the aid of a walker due to severe pain and weakness. She had eighteen out of eighteen Fibromyalgia tender points detected by mild digital pressure, muscle spasms in the cervical and lumbar paraspinal muscles and spasms in both shoulders. Subsequently she was placed on Oxycodone 20 mg P.O. q8-12 hrs, acetaminophen 750/Hydrocodone 7.5 mg, 1-2 Tabs P.O. q 4 hr, prn pain, and Baclofen 10 mg,/½ tab P.O., tid. In the following months she had trigger point injections using local anesthetics and steroid, and also denervation of peripheral nerves and muscles in spasms using Botulinum toxin. After each of these procedures the patient's pain score would drop from a score of 10/10 to 4-5/10 within three days. The relief would persist for several weeks before pain will gradually increase to a score of 10/10. During an exacerbation of the patient's condition, she was treated with intra-venous infusion of methylprednisolone succinate 125 mg. This resulted in a dramatic pain relief with associated resolution of fatigue. The pain dropped to a score of 2/10 as never before, and muscle spasms were mild and infrequent.

Mrs. K.

64-year-old female presented with a fifteen-year history of low back pain and severe pain in the tailbone, which started after a slip and fall on the buttocks. Examination of the spine revealed marked tenderness in the lumbar spinous processes and paraspinal muscles as well as the coccyx. MRIs of the lumbar spine, sacrum and coccyx were ordered. These revealed multiple-level diffuse disc bulge in the lumbar spine measuring 3-4 mm with displacement of the posterior longitudinal ligament, neural foramina narrowing and disk desiccations. However, there were no signs of fracture in the lumbar spine, sacrum and coccyx. She has had two lumbar spine epidurals, several trigger point injections using local anesthetic and steroid, in addition to hydromorphone 4 mg, 1-2 tabs P.O. q4 hr, prn pain, morphine SR 60 mg P.O q12 hr, and Rofecoxib 50 mg P.O. qd. These treatments only resulted in moderate and transient pain relief in the lumbar region. The pain over the coccyx persisted until the patient was given Etanercept injection 25 mg subcutaneously. Within two days, the patient had significant relief of pain in the tailbone and lower back. Her requirement for oral hydromorphone 4 mg decreased from six tablets daily with three tablets of morphine SR 60 mg to just one tablet of hydromorphone 4 mg. The pain relief was significant and lasted one month before gradually increasing to the pre-Etanercept injection levels.

Mr. L.

A 37-year old male presents with a 9-year history of pain in the right and left upper extremities. Pain had started gradually in the right hand few months after patient had repeated surgeries in the right hand for open reduction and reconstruction of multiple fractures in the fingers of the right hand following a fall. Pain is described as a severe (score of 10 on a numeric scale of 1 to 10), constant burning pain with associated numbness and tingling. There is also associated swelling of the right hand with bluish discoloration and mottled appearance of the skin. In addition, the patient felt the right hand to be always cold. The patient does not report increased sweatiness in the hands, but stated that the right hand is very sensitive to touch; light contact is enough to cause pain. The patient reports that the pain in the right hand had gradually progressed, spreading upward to involve the entire right upper extremity and later spreading to the left upper extremity. The pain in the left upper extremity is less severe compared to the right upper extremity. Prior to presentation, the patient has had a cervical sympathetic block without relief. Patient said that the block was complicated by Carotid artery edema causing severe neck pain and migraine headaches. The patient's past medical history is significant for subacute bacterial endocarditis, mitral valve stenosis, and Premature Ventricular Complexes precipitated by epinephrine, ephedrine or medications containing ephedrine, and codeine. The patient also has a history of hyperventilation and panic attacks. The patient's current medication list includes alprazolam 3 mg daily and hydrocodone 10 mg/acetaminophen 325 mg, 10 tablets daily. The patient works as a system engineer and has had to be placed on restricted duties due to his present condition. The patient said he has had to learn to use his left hand more due to the severe pain in his right hand.

Physical examination revealed scars from previous injury and surgeries in the right index and fourth finger, mottled hyperemia of the right hand, reduced temperature of the right hand compared to the left (temperature difference of 2.1 degrees noted using an Infrared Temperature Scanner), mild non-pitting edema of the right hand, severe tenderness of the right hand and right forearm with reduced right hand grip (Right Hand Grip Strength: 0 psi, Left Hand Grip Strength: 10 psi). Additional findings on physical examination were moderate tenderness in the right cervical paraspinal muscles, right upper Trapezius and right Supraspinatus muscles with mild reduction in cervical flexion (40/45), shoulder range of motion was also slightly reduced. There was also hyperesthesia of the right hand and right forearm.

The patient's diagnosis is Reflex Sympathetic Dystrophy, also known as Complex Regional Pain Syndrome. Initial treatment regimen consisted of methylprednisolone 125 mg IVP, dexamethasone 6 mg IVP, oxcarbazepine 300 mg po bid, methadone 5 mg po q 6 hrs, hydrocodone 10 mg/acetaminophen 325 mg 1 to 2 tablets po q 6 hrs, prn pain, maximum 8 tablets daily. In addition, the patient was given a compounded topical preparation of lidocaine 5%, diclofenac 10%, ketamine 3%, gabapentin 10% and dexamethasone 0.1% to apply 2 to 3 times daily. On re-evaluation one week later, the patient's condition was significantly better with his right hand pain now becoming mild to moderate in intensity (pain score ranging from 4 to 6 on the numeric scale). The pain in the left upper extremity had completely resolved. Furthermore, the patient was able to tolerate increased levels of physical activity without commensurate increase in amount of pain. An infusion of pamidronate was recommended but the patient declined due to his fears that it might cause him to have Premature Ventricular Complexes. The patient was later put on alendronate sodium 70 mg po once weekly and dose of oxcarbazepine increased to 600 mg po bid. Over a 6-month period, the patient received eight doses of etanercept 25 mg S.Q and six doses of anakinra 100 mg SQ alternately at intervals of one to two weeks depending on the patient's condition. The patient also had three doses of methylprednisolone 125 mg IVP and dexamethasone 4 mg IVP over the six-month period.

On re-evaluation at six months since commencing treatment, the patient's right hand pain was mild, pain score ranging from ½ to 1 with occasional numbness and tingling. The patient could tolerate increased level of physical activity at work and had resumed his full duties at work. The patient was very pleased with the result of the treatment and he felt his life was back on track. On examination, patient had mild tenderness in the right hand with minimal mottling and a temperature difference of 1.1 degrees.

Conclusion

In accordance with Sota Omoigui's Law of Pain, the origins of pain are the biochemical mediators of inflammation and the inflammatory response. To treat pain, we must block these mediators and block the signals they send up through the nerve cells.

It will be apparent to those skilled in the art that variations and modifications to the specific embodiments disclosed herein may be made without departing from the scope of the invention.

### REFERENCES

¹Sherrington C S. The Integrative Action of the Nervous System. New York: Scribner, 1906
²W. K. Livingstone. Pain Mechanisms. New York: Macmillan, 1943.
³Online Exhibit. Pain and Suffering in History-Narratives of Science, Medicine and Culture. John C. Liebeskind History of Pain Collection at the Louise M. Darling Biomedical Library, UCLA.
⁴Science 1965 November 19;150(699):971-9 Pain mechanisms: a new theory. Melzack R, Wall P D.
⁵Pain. 1979 June; 6 (3):249. Pain terms: a list with definitions and notes on usage. Recommended by the IASP Subcommittee on Taxonomy. [No authors listed]
⁶Don Ranney, MD, FRCS. Anatomy of Pain: Paper presented at the Ontario Inter-Urban Pain Conference. Waterloo. 1996
⁷Boivie J, Central pain syndromes. In Campell J N (ed) Pain 1996—An Updated Review, IASP Press, Seattle, p.23-29.
⁸Dubner R, Basbaum A, Spinal dorsal horn plasticity following tissue or nerve injury. Chapter 11 in Walls P D and Melzack R (eds): Textbook of Pain, 3rd ed., 1994, Churchill livingstone, Edinburgh, p.225-241
⁹Jensen T S Mechanisms of neuropathic pain. In Campbell J N (ed) Pain 1996—An Updated Review, IASP Press, Seattle, p.77-86.
¹⁰Merskey H M, Bogduk N, Classification of Chronic Pain, 2nd ed., 1994 IASP Press, Seattle, p.211
¹¹ J Bone Joint Surg Am 1990 March;72(3):403-8 Abnormal magnetic-resonance scans of the lumbar spine in asymptomatic subjects. A prospective investigation. Boden S D, Davis D O, Dina T S, Patronas N J, Wiesel S W.
¹² N Engl J Med 1994 July 14;331(2):69-73 Magnetic resonance imaging of the lumbar spine in people without back pain. Jensen M C, Brant-Zawadzki M N, Obuchowski N, Modic M T, Malkasian D, Ross J S.
¹³Natural history of individuals with asymptomatic disc abnormalities in magnetic resonance imaging: predictors of low back pain-related medical consultation and work incapacity. Boos N, Semmer N, Elfering A, Schade V, Gal I, Zanetti M, Kissling R, Buchegger N, Hodler J, Main C J.
¹⁴N Engl J Med 347: 81-88. A controlled trial of arthroscopic surgery for osteoarthritis of the knee. Moseley J B, O'Malley K, Petersen N J, Menke T J, Brody B A, Kuykendall D H, Hollingsworth J C, Ashton C M, Wray N P
¹⁵Arthritis Res 2000 2: 145-153 Active synovial matrix metalloproteinase-2 is associated with radiographic erosions in patients with early synovitis Raphaela Goldbach-Mansky¹, Jennifer M Lee¹, Joseph M Hoxworth¹, David Smith II¹, Paul Duray¹, Ralph H Schumacher Jr¹, Cheryl H Yarboro¹, John Klippel¹, David Kleiner¹ and Hani S El-Gabalawy¹
¹⁶Mol Cell Biol 1989 May;9(5):2239-43 Effects of botulinum toxin type D on secretion of tumor necrosis factor from human monocytes. Imamura K, Spriggs D, Ohno T, Kufe D.
¹⁷Lab Invest 2001 August;81(8):1109-17 Inhibitors of Phosphodiesterase Isoforms III or IV Suppress Islet-Cell Nitric Oxide Production. Beshay E, Prud'homme G J.
¹⁸Spine 2001 April 15;26(8):870-5 Prevention of compartment syndrome in dorsal root ganglia caused by exposure to nucleus pulposus. Yabuki S, Onda A, Kikuchi S, Myers R R.
¹⁹Proc Natl Acad Sci U S A 1996; 93: 14014-9. A novel mechanism of action of tetracyclines: effects on nitric oxide syntheses Amin A R, Attur M G, Thakker G D, Patel P D, Vyas P R, Patel R N, Patel I R, Abramson S B.
²⁰Biochem Pharmacol 1998; 55: 93-100 Pharmacological effect of tetracyclines on proteoglycanases from interleukin-1-treated articular cartilage Steinrneyer J, Daufeldt S, Taiwo Y O.
²¹Clin EU Immunol 1995; 102: 635-41. The influence of tetracyclines on T cell activation, Kloppenburg M, Verweij C L, Miltenburg A M, Verboeven A J, Daha M R, Dijlanans B A, Breeveld F C.
²²J Periodontal Res 1997; 32: 183-8 Tetracycline inhibits' Porphyromonas gingivalis lipopolysaccharide-induced lesions in vivo and TNF .alpha. processing in vitro. Shapira L, Houri Y, Barak V, Soskolne W A, Halabi A, Stabholz A.
²³Chao C C, Hu S, Ehrlich L, Peterson P K, Interleukin-1 and tumor necrosis factor-alpha synergistically mediate neurotoxicity: involvement of nitric oxide and of N-methyl-D-aspartate receptors, Brain Behav Immun 1995; 9: 355-65
²⁴Bluthe R M, Dantzer R, Kelley K W, Interleukin-I mediates behavioural but not metabolic effects of tumor necrosis factor alpha in mice, Eur J Pharmacol 1991; 209: 281-3.
²⁵Hartung B P, Jung S, Stoll G, Zielasek J, Schmidt B, Archelos J J, Toyka K V, Inflammatory mediators in demyelinating disorders of the CNS and PNS, J Neuroinununol 1992; 40: 197-210.
²⁶Sommer C, Schmidt C, George A, Toyka K V, A metalloprotease-inhibitor reduces pain associated behaviour in mice with experimental neuropathy, Neurosci Lett 1997;237:45-8
²⁷Stoll G, Jung S, Jander S, van der Meide P, Hartung B P, Tumor necrosis factor-alpha in immunomediated demyelination and Wallerian degeneration of the rat peripheral nervous system, Neuroimmunol 1993; 45: 175-82.
²⁸Scand J Rheumatol Suppl 2000;113:37-45 Preclinical and clinical pharmacology of the 5-HT3 receptor antagonists. Wolf H.
²⁹Hematology (Am Soc Hematol Educ Program) 2000;:147-165 Multiple Myeloma: New Insights and Therapeutic Approaches.
³⁰Psychoneuroendocrinology 2000 November;25(8):785-97 The effect of repeated amitriptyline and desipramine administration on cytokine release in C57BL/6 mice. Kubera M, Holan V, Mathison R, Maes M.
³¹Neuroscience 1999;91(1):211-9 Corticosterone and phenytoin reduce neuronal nitric oxide synthase messenger RNA expression in rat hippocampus. Reagan L P, McKittrick C R, McEwen B S.
³²Res Commun Mol Pathol Pharmacol 1999;105(1-2):23-33 Zonisamide inhibits nitric oxide synthase activity induced by N-methyl-D-aspartate and buthionine sulfoximine in the rat hippocampus. Noda Y, Mori A, Packer L.
³³Pain 2000 December 1;88(3):267-75 Thalidomide treatment in chronic constrictive neuropathy decreases endoneurial tumor necrosis factor-alpha, increases interleukin-10 and has long-term effects on spinal cord dorsal horn met-enkephalin. George A, Marziniak M, Schafers M, Toyka K V, Sommer C.
³⁴Br J Anaesth 1995 August;75(2):125-31 Inflammatory mediators of pain. Dray A.
³⁵Clin J Pain 1991;7 Suppl 1:S8-15 Pathophysiological mechanisms of fibromyalgia. Zimmermann M.
³⁶ Nature 410, 471-475 (2001) © Macmillan Publishers Ltd. Interleukin-1-mediated induction of Cox-2 in the CNS contributes to inflammatory pain hypersensitivity Tarek A. Samad, Kimberly A. Moore, Adam Sapirstein, Sara Billet, Andrew Allchorne, Stephen Poole, Joseph V. Bonventre & Clifford J. Woolf
³⁷Eur J Neurosci 1999 March;11(3):837-46 Adrenergic innervation of rat sensory ganglia following proximal or distal painful sciatic neuropathy: distinct mechanisms revealed by anti-NGF treatment. Ramer M S, Bisby M A.
³⁸Hyperalgesia from subcutaneous cytokines By Stephen Poole, Fernando de Queiroz Cunha and Sergio Henriques Ferreira Summary
³⁹J Exp Med. 2000;191:313-320. Development of chronic inflammatory arthropathy resembling rheumatoid arthritis in interleukin 1 receptor antagonist-deficient mice. Horai R, Saijo S, Tanioka H, et al.
⁴⁰Adv Immunol. 1993;54:167-227. Interleukin-1 receptor antagonist. Arend W P.
⁴¹J Rheumatol. 1995;22:2250-2258. Major role for interleukin 1 but not for tumor necrosis factor in early cartilage damage in immune complex arthritis in mice. van Lent P L E M, van de Loo F A J, Holthuysen A E M, van den Bersselaar L A M, Vermeer H, van den Berg W B.
⁴²J. Exp Med. 2000;191:313-320 Development of chronic inflammatory arthropathy resembling rheumatoid arthritis in interleukin 1 receptor antagonist-deficient mice. Horai R, Saijo S, Tanioka H, et al.
⁴³ Arthritis Rheum. 2000;43:2143-2151. Cellular mechanisms and the role of cytokines in bone erosions in rheumatoid arthritis. Gravallese E M, Goldring S R.
⁴⁴J Rheumatol. 1995;22:2250-2258. Major role for interleukin 1 but not for tumor necrosis factor in early cartilage damage in immune complex arthritis in mice. van Lent P L E M, van de Loo F A J, Holthuysen A E M, van den Bersselaar L A M, Vermeer H, van den Berg W B.
⁴⁵In: Interleukin-6-type Cytokines. (A. Mackiewicz, A. Koji and P. B. Sehgal, eds.), Vol. 762, pp. 1-14. New York Academy of Sciences, New York 1995 Interleukin-6 type Cytokines. P. B. Sehgal, L. Wang, R. Rayanade, H. Pan & L Margulies:
⁴⁶Frontiers in Bioscience 1, d340-357, Dec. 1, 1996 Molecular and cellular biology of interleukin-6 and its receptor. Evan T. Keller Jon Wanagat, W. B. Ershler
⁴⁷ Adv Immunol 54, 1-78 (1993) Interleukin-6 in biology and medicine. S. Akira, T. Taga & T. Kishimoto:
⁴⁸J Clin Invest 95, 2846-2852 (1995) Interleukin-6 enhances hypercalcemia and bone resorption mediated by parathyroid hormone-related protein in vivo. J. De La Mata, H. L. Uy, T. A. Guise, B. Story, B. F. Boyce, G. R. Mundy & G. D. Roodman:
⁴⁹Frontiers in Bioscience, 1, d340-357 Molecular and cellular biology of interleukin-6 and its Receptor. Evan T. Keller, Jon Wanagatl, W. B. Ershler
⁵⁰ J Am Geriatr Soc 41, 176-181 (1993 Interleukin-6: A cytokine for gerontologists. W. B. Ershler:
⁵¹Scientific American 274, 46-52 (1996) Caloric restriction and aging. R. Weindruch:
⁵²Cancer Res 54, 3054-3063 (1993) Dietary restriction from middle age attenuates ageassociated lymphoma development and IL-6 dysregulation in C57BL/6 mice. M. J. Volk, T. D. Pugh, M. J. Kim, C. H. Frith, R. A. Daynes, W. B. Ershler & R. Weindruch:
⁵³ Lymphokine Cytokine Res 12, 225-230 (1993) Interleukin-6 and aging: blood levels and mononuclear cell production increase with advancing age and in vitro production is modifiable by dietary restriction. W. B. Ershler, W. H. Sun, N. Binkley, S. Gravenstein, M. J. Volk, G. Kamoske, R. G. Klopp, E. B. Roecker, R. A Daynes & R. Weindruch:
⁵⁴ Ann NY Acad Sci 521, 260-273 (1988) Hormonal intervention: “buffer hormones” or “state dependency”. The role of dehydroepiandrosterone (DHEA), thyroid hormone, estrogen and hypophysectomy in aging. W. Regelson, R. Loria & M. Kalimi:
⁵⁵ J Immunol 150, 5219-5230 (1993) Altered regulation of IL-6 production with normal aging. Possible linkage to the age-associated decline in dehydroepiandrosterone and its sulfated derivative. R. A Daynes, B. A. Araneo, W. B. Ershler, C. Maloney, G. Z. Li & S. Y. Ryu
⁵⁶ J Exp Med 67, 332-344 (1988) The essential role of B cell stimulatory factor 2 (BSF-2/IL-6) for the terminal differentiation of B cells. A. Muraguchi, T. Hirano, B. Tang, T. Matsuda, Y. Horii, K. Nakamima & T. Kishimoto:
⁵⁷ J Exp Med 167, 1414-1427 (1988) T cell growth and differentiation induced by interleukin HP1/IL-6, the murine hybridoma/plasmacytoma growth factor. C. Uyttenhove, P. G. Coulie & J. Van Snick
⁵⁸ Eur J Immunol 18, 957-959 (1988) Interleukin-6 is involved in interleukin-1-induced activities. M. Helle, J. P. J. Brakenhoff, E. T. DeGroot & L. A Aarden:
⁵⁹. J Immunol 141, 1543-1549 (1988) IL-6/BSF-2 functions as a killer helper factor in the in vitro induction of cytotoxic T cells M. Okada, N. Kitahara, S. Kishimoto, T. Matsuda, T. Hirano & T. Kishimoto
⁶⁰ J Immunol 143, 1894-1898 (1989) IL1 and IL-6 in CTL induction-accessory signals in murine cytolytic T cell responses: Dual requirement for IL1 and IL6. J. C. Renauld, A. Vink & J. Van Snick
⁶¹ J Immunol 143, 1206-1209 IFNb/IL-6 augments the activity of human natural killer cells. T. A. Luger, J. Krutman, T. Kirnbauer, A. Urbanski, T. Schwarz, G. Klappacher, A. Kock, M. Micksche, K. J. Malejczy, E. Schauer, L. T. May, & P. B. Sehgal
⁶² J Immunol 144, 4226-4230 (1990) IL-6 stimulates osteoclast-like multinucleated cell formation in long term human marrow cultures by inducing IL-1 release. N. Kurihara, D. Bertolini, T. Suda, Y. Akiyama & G. D. Roodman:
⁶³ Proc Natl Acad Sci USA 90, 11924-11928 (1993) Soluble interleukin-6 receptor triggers osteoclast formation by interleulin-6. T. Tamura, N. Udagawa, N. Takahashi, C. Miyaura, S. Tanaka, Y. Koishihara, Y. Ohsugi, K. Kumali, T. Taga, T. Kishimoto & T. Suda:
⁶⁴ J Bone Miner Res 6, S263 (1991) 17b-estradiol regulates IL-6 production and osteoclast development in murine calvaria cell cultures. G. Passeri, G. Girasole, T. Markus, J. S. Abrams, S. C. Manolagas & R. L. Jilka
⁶⁵.J Clin Invest 89, 883-891 (1992) 17b-Estradiol inhibits interleulin-6 production by bone marrow-derived stromal cells and osteoblasts in vitro: a potential mechanism for the antiosteoporotic effect of estrogens G. Girasole, R. L. Jilka, F. Passeri, S. Boswell, G. Boder, D. C. Williams & S. C. Manologas
⁶⁶ Science 257, 88-91 (1992) Increased osteoclast development after estrogen loss: Mediation by interleukin-6. R. L. Jilka, C. Hangoc, G. Girasole, G. Passeri, D. C. Williams, J. S. Abrams, B. Boyce, H. Broxmeyer & S. C. Manolagas
⁶⁷ J Bone Miner Res 10, 1365-1373 (1995) Endogenous bone resorbing factors in estrogen deficiency: Cooperative effects of IL-1 and IL-6 C. Miyaura, K Kusano, T. Masuzawa, 0. Chaki, Y. Onoe, M. Aoyagi, T. Sasaki, T. Tamura, Y. Koishihara, Y. Ohsugi & T. Suda:.
⁶⁸ J Bone Miner Res 6, S271 (1991) Interleukin-6 causes hypercalcemia in vivo, and enhances the bone resorbing potency of interleukin-1 and tumor necrosis factor by two orders of magnitude in vitro. K. S. Black, G. R. Mundy & I. R. Garrett
⁶⁹J Clin Invest 96, 1238-1244 (1995) Adenyl cyclase and interleukin 6 are downstream effectors of parathyroid hormone resulting in stimulation of bone resorption. E. M. Greenfield, S. M. Shaw, S. A. Gornik & M. A. Banks
⁷⁰ Biochem Biophys Res Comm 162, 1546-1552 (1989) Parathyroid hormone (PTH) and PTHlike protein (PLP) stimulate interleukin-6 production by osteogenic cells: A possible role of interleulin-6 in osteoclastogenesis. C. W. G. M. Löwik, G. van der Pluijm, H. Bloys, K. Hoekman, O. L. M. Bijvoet, L. A. Asrden & S. E. Papapoulos
⁷¹ J Immunol 145, 3297-3303 (1990) IL-6 is produced by osteoblasts and induces bone resorption. Y. Ishimi, C. Miyaura, C. H. Jin, T. Akatsu, E. Abe, Y. Nakamura, A. Yamaguchi, S. Yoshiki, T. Matsuda, T. Hirano, T. Kishimoto & T. Suda
⁷²J Clin Invest 89, 46-52 (1992) Interleukin-6: a potential autocrine/paracrine factor in Paget's disease of Bone. G. D. Roodman, N. Kurihara, Y. Ohsaki, T. Kulita, D. Hosking, A. Demulder & F. R. Singer
⁷³Y. Ohsaki, S. Takahashi, T. Scarcez, A Demulder, T. Nishihara, R. Williams & G. D. Roodman: Evidence for an autocrine/paracrine role for interleukin-6 in bone resorption by giant cells from giant cell tumors of bone. Endocrinology 131, 2229-2234 (1992)
⁷⁴ J. De La Mata, H. L. Uy, T. A. Guise, B. Story, B. F. Boyce, G. R. Mundy & G. D. Roodman: Interleukin-6 enhances hypercalcemia and bone resorption mediated by parathyroid hormone-related protein in vivo. J Clin Invest 95, 2846-2852 (1995) ⁷⁵ J Biochem (Tokyo). 1999 September;126(3):553-8. Interleulin-1beta induces interleukin-6 production through the production of prostaglandin E(2) in human osteoblasts, MG-63 cells. Takaoka Y, Niwa S, Nagai H.
⁷⁶S. Tabibzadeh, Q. F. Kong, A. Babaknia & L. T. May: Progressive rise in the expression of intereleukin-6 in human endometrium during menstrual cycle is initiated during the implantation window. Mol Hum Reprod 1, 2793-2799 (1995) ⁷⁷ T. Hama, M. Miyamoto, H. Tsukui, C. Nishio & M. Hatanaka: Interleukin-6 as a neurotrophic factor for promoting the survival of cultured basal forebrain cholinergic neurons from postnatal rats. Neurosci Lett 104, 340-344 (1989)
⁷⁸ Brain Res 2000 October 6;879(1-2):216-25 Intrathecal anti-IL-6 antibody and IgG attenuates peripheral nerve injury-induced mechanical allodynia in the rat: possible immune modulation in neuropathic pain. Arruda J L, Sweitzer S, Rutkowski MD, Deleo J A.
⁷⁹S. Kotake, K Sato, K. J. Kim, N. Takahashi, N. Udagawa, I. Nakamura, A. Yamaguchi, T. Kishimoto, T. Suda & S. Kashiwazaki: Interleulin-6 and soluble interleukin-6 receptors in the synovial fluids form rheumatoid arthritis patients are responsible for osteoclast-like cell formation. J Bone Miner Res 11, 88-95 (1996)
⁸⁰F. Houssiau, J. P. Devoglaer, J. Van Damme, C. Nagant de Deuxchaisnes & J. Van Snick: Interleukin 6 in synovial fluid and serum of patients with rheumatoid arthritis and other inflammatory arthritides. Arthritis Rheum 31, 784-788 (1988)
⁸¹ Intrathecal Methylprednisolone for Intractable Postherpetic Neuralgia Naoki Kotani, Tetsuya Kushikata, Hiroshi Hashimoto, Futoshi Kimura, Masatoshi Muraoka, Misako Yodono, Mizue Asai, Akitomo Matsuki
⁸² J Neurotrauma 1999 October;16(10):851-63 Systemically administered interleukin-10 reduces tumor necrosis factor-alpha production and significantly improves finctional recovery following traumatic spinal cord injury in rats. Bethea J R, Nagashima H, Acosta M C, Briceno C, Gomez F, Marcillo A E, Loor K, Green J, Dietrich W D.
⁸³ Exp Neurol 2001 March;168(1):144-54 Effects of interleulin-10 (IL-10) on pain behavior and gene expression following excitotoxic spinal cord injury in the rat. Plunkett J A, Yu C G, Easton J M, Bethea J R, Yeziersid R P.
⁸⁴Kayama S, Konno S, Olmarker K, Yabuki S, Kikuchi S, Incision of the anulus fibrosis induces nerve root morphologic, vascular, and functional changes. An experimental study, Spine 1996; 21: 2539-43.
⁸⁵Olmarker K, Rydevik B, Nordborg C, Autologous nucleus pulposus induces neurophysiologic and histologic changes in porcine cauda equina nerve roots, Spine 1993; 18: 1425-32.
⁸⁶Olmarker K, Myers R R, Pathogenesis of sciatic pain: Role of herniated nucleus pulposus and deformation of spinal nerve root and DRG, Pain, 1998, 78: 9-105
⁸⁷ Hyperalgesic Actions of Cytokines on Peripheral Nerves By Robert R. Myers, Rochelle Wagner and Linda Sorkin
⁸⁸P Borden, R A Heller: Transcriptional control of matrix metalloproteinases and the tissue inhibitors of matrix metalloproteinases Crit Rev Eukaryot Gene Expr 1997, 7: 159-178
⁸⁹C He: Molecular mechanism of transcriptional activation of human gelatinase B by proximal promoter Cancer Lett 1996, 106: 185-191
⁹⁰ Active synovial matrix metalloproteinase-2 is associated with radiographic erosions in patients with early synovitis Raphaela Goldbach-Mansky¹, Jennifer M Lee¹, Joseph M Hoxworth¹, David Smith II¹, Paul Duray¹, Ralph H Schumacher Jr¹, Cheryl H Yarborol, John Klippel¹, David Kleiner¹ and Hani S El-Gabalawy¹ Arthritis Res 2000 2: 145-153
⁹¹ J Neurosci 1996 December 15;16(24):7910-9 Matrix metalloproteinase-9 (MMP-9) is synthesized in neurons of the human hippocampus and is capable of degrading the amyloid-beta peptide (1-40). Backstrom J R, Lim G P, Cullen M J, Tokes Z A.
⁹² Pain 2000 February;84(2-3):159-67 Cytokine involvement in dynorphin-induced allodynia. Laughlin T M, Bethea J R, Yezierski R P, Wilcox G L.
⁹³ Lakartidningen 1997 November 26;94(48):4461-6 [A breakthrough in the research on pain. Survey of the synaptic network may result in new analgesics] Olgart L.
⁹⁴Merck Medicus Disease Modules: Osteoarthritis. March 2001. Merck and Co Inc.
⁹⁵Dequeker J, Dieppe P A, eds. Disorders of bone cartilage and connective tissue. In: Klippel J H, Dieppe P A, eds. Rheumatology. 2nd ed. London: Mosby, 1998
⁹⁶ American College of Rheumatology. Clinical Slide Collection on the Rheumatic Diseases. Atlanta: American College of Rheumatology, 1997
⁹⁷ Rev Prat 1994 June 15;44(12):1569-71 Substance P and rheumatic diseases Menkes C J, Renoux M.
⁹⁸ Inflammation 1992 December;16(6):587-601 Effects of interleukin-1 beta and tumor necrosis factor-alpha on osteoblastic expression of osteocalcin and mineralized extracellular matrix in vitro. Taichman R S, Hauschka P V.
⁹⁹ Calcif Tissue Int 1993 March;52(3):227-33 Proliferative responses to estradiol, IL-1 alpha and TGF beta by cells expressing alkaline phosphatase in human osteoblast-like cell cultures. Rickard D J, Gowen M, MacDonald B R.
¹⁰⁰ Arthritis Rheum 2001 October;44(10):2320-30 In vivo dual inhibition of cyclooxygenase and lipoxygenase by ML-3000 reduces the progression of experimental osteoarthritis: suppression of collagenase 1 and interleukin-1beta synthesis. Jovanovic D V, Fernandes J C, Martel-Pelletier J, Jolicoeur F C, Reboul P, Laufer S, Tries S, Pelletier J P.
¹⁰¹Whyte J J. Osteoporosis: Diagnosis and Treatment. CME resource: course #9901 2004:1-15 ¹⁰² Iqbal M M. Osteoporosis: epidemiology, diagnosis and treatment. Southern Medical Journal 2000;93:2-18
¹⁰³Studies of Human Osteoblast-Like Cells—Effects of Growth Hormone and Steroids, eJIFCC vol 13 no 4: Diana Swolin-Eide, M. D., Ph.D.
¹⁰⁴ Osteoporosis and Bone Physiology. Ott S. M.
¹⁰⁵Melton, L. J.(1995). How many women have osteoporosis now? J Bone Miner Res 10: 175-7.
¹⁰⁶ Nelson, H. D.(2002). Screening for postmenopausal osteoporosis: a review of the evidence for the U.S. Preventive Services Task Force. Ann Intern Med 137: 529-41.
¹⁰⁷ Ann Intern Med. 1998 May 15;128(10):793-800. The association of radiographically detected vertebral fractures with back pain and function: a prospective study. Nevitt M C, Ettinger B, Black D M, Stone K, Jamal S A, Ensrud K, Segal M, Genant H K, Cummings S R
¹⁰⁸ Tannenbaum C, Clark J, Schwartzman K, Wallenstein S, Lapinski R, Meier D, Luckey M. Yield of laboratory testing to identify secondary contributors to osteoporosis in otherwise healthy women. J Clin Endocrinol Metab. 2002 October;87(10):4431-7.
¹⁰⁹Arthritis & Rheumatism Volume 39, No. 11, November 1996, pp 1791-1801 Treatment of Steroid-Induced Osteoporosis Acr Task Force on Osteoporosis Guidelines
¹¹⁰ J Clin Invest. 1999 November;104(10):1363-74. Prevention of osteocyte and osteoblast apoptosis by bisphosphonates and calcitonin. Plotkin L I, Weinstein R S, Parfitt A M, Roberson P K, Manolagas S C, Bellido T
¹¹¹Dempster D W, Arlot M A, Meunier P I: Mean wall thickness and formation periods of trabecular bone packets in corticosteroid-induced osteoporosis. Calcif Tissue Int 35:410-417, 1983
¹¹²Gronowicz G, McCarthy M B: Glucocorticoids inhibit the attachment of osteoblasts to bone extracellular matrix proteins and decreases [gb]-1 integrin levels. Endocrinology 136:598-608, 1995
¹¹³Hughes-Fulford M, Blaug S: Down regulation of c-fos and cell growth with upregulation of p53 oncogene in glucocorticoid treated osteoblasts (abstract). J Bone Miner Res 8:S365, 1993
¹¹⁴Canalis E M: Effect of glucocorticoids on type I collagen synthesis, alkaline phosphatase activity, and deoxyribonucleic acid content in cultured rat calvariae. Endocrinology 112:931-939, 1983
¹¹⁵Jonsson K B, Ljunghall S, Karlstrom O, Johansson A G, Mallmain H, Ljunggren O: Insulin-like growth factor I enhances the formation of type I collagen in hydrocortisone-treated human osteoblasts. Biosci Rep 13:297-302, 1993
¹¹⁶Centrella M, McCarthy T L, Canalis E: Glucocorticoid control of transforming growth factor [gb] activity and binding and effects in osteoblast-enriched cultures from fetal rat bone. Mol Cell Biol 11:4490-4496, 1991
¹¹⁷Studies of Human Osteoblast-Like Cells—Effects of Growth Hormone and Steroids, eJIFCC vol 13 no 4: Diana Swolin-Eide, M.D., Ph.D.
¹¹⁸Klein R G, Arnaud S B, Gallagher J C, Deluca H F, Riggs B L: Intestinal calcium absorption in exogenous hypercortisolism: role of 25-hydroxyvitamin D and corticosteroid dose. J Clin Invest 64:655-665, 1977
¹¹⁹Hahn T J, Halstead L R, Teitelbaum S L, Hahn B H: Altered mineral metabolism in glucocorticoid-induced osteopenia: effect of 25-hydroxy vitamin D administration. J Clin Invest 64:655-665, 1979
¹²⁰Suzulki Y, Ichikawa Y, Saito E, Homma M: Importance of increased urinary calcium excretion in the development of secondary hyperparathyroidism of patients under glucocorticoid therapy. Metabolism 32:151-156, 1983
¹²¹Brandli D W, Golde G, Greenwald M, Silverman S L: Glucocorticoid-induced osteoporosis: a cross-sectional study. Steroids 56:518-523, 1991
¹²²Hahn T J, Halstead L R, Teitelbaum S L, Hahn B H: Altered mineral metabolism in glucocorticoid-induced osteopenia: effect of 25-hydroxy vitamin D administration. J Clin Invest 64:655-665, 1979
¹²³Suzuki Y, Ichikawa Y, Saito E, Homma M: Importance of increased urinary calcium excretion in the development of secondary hyperparathyroidism of patients under glucocorticoid therapy. Metabolism 32:151-156, 1983
¹²⁴Hahn T J, Halstead L R, Teitelbaum S L, Hahn B H: Altered mineral metabolism in glucocorticoid-induced osteopenia: effect of 25-hydroxy vitamin D administration. J Clin Invest 64:655-665, 1979
¹²⁵Seeman E, Kumar R, Huder G G, Scott M, Heath H, Riggs B L: Production, degradation and circulating levels of 1,25-dihydroxy vitamin D in health and in chronic glucocorticoid excess. J Clin Invest 66:664-669, 1980
¹²⁶ Hsueh A J, Erickson G F: Glucocorticoid inhibition of FSH-induced estrogen production in cultured rat granulosa cells. Steroids 32:639-648, 1978
¹²⁷ MacAdams M R, White R H, Chipps B E: Reduction in serum testosterone levels during chronic glucocorticoid therapy. Ann Intern Med 104:648-651, 1986
¹²⁸Reid I R, France J T, Pybus J, Ibbertson H K: Low plasma testosterone levels in glucocorticoid-treated male asthmatics. BMJ 291:574-578, 1985
¹²⁹p. Navarra, S. Tsagarakis, M. Faria, L. H. Rees, M. Besser & A. B. Grossman: Interleukin-1 and -6 stimulate the release of corticotropoin-releasing hormone from rat hypothalamus in vitro via eicosanoid cyclooxygenase pathway. Endocrinology 128, 37-44 (1990)
¹³⁰K Lyson, K. Milenkovic & S. M. McCann: The stimulatory effect of interleukin-6 on corticotropin releasing factor and thyrotropin-releasing hormone secretion in vitro. Prog Neuroendocrinol Immunol 4, 161-165 (1991)
¹³¹B. M. R. N. J. Woloski, E. M. Smith, W. J. Meyer III, G. M. Fuller & J. E. Blalock: Corticotropin-releasing activity in monokines. Science 230, 1035-1037 (1985)
¹³²M. Kohase, D. Henriksen-DiStefano, L. T. May & J. Vilcek: Dexamethasone inhibits feedback regulation of the mitogenic activity of tumor necrosis factor, interleulin-1 and epidermal growth factor in human fibroblasts. J Cell Physiol 132, 271-278 (1987)
¹³³H. J. M. Feyen, P. Elford, F. E. Ki Padova & U. Trechsel: Interleukin-6 is produced by bone and modulated by parathyroid hormone. J Bone Miner Res 4, 633-638 (1989)
¹³⁴T. Nishida, S. Nakai, T. Kawakami, K. Aihara, N. Nishino & Y. Hirai: Dexamethasone regulation of the expression of Cytokine mRNAs induced by interleukin-1 in the astrocytoma cell line U373MG. FEBS Lett 243, 25-29 (1989)
¹³⁵Prevention and Treatment of Glucocorticoid-induced Osteoporosis. The National Preferred Medicines Centre Incorporated. Number 79 May 1999
¹³⁶ Nippon Rinsho. 2003 February;61(2):292-8. Osteoporosis associated with rheumatoid arthritis Kameda H, Takeuchi T.
¹³⁷Interleukin-6 deficient mice are protected from bone loss caused by estrogen depletion. EMBO Journal 1994 March 1;13 (5):1189-96 Poli V, Balena R, Fattori E, Markatos A, Yamamoto M, Tanaka H, Ciliberto G, Rodan G A, Costantini F
¹³⁸S. S. Tabibzadeh, U. Santhanam, P. B. Sehgal & L. T. May: Cytokine-induced production of IFN-132/IL-6 by freshly explanted human endometrial stromal cells. Modulation by estradiol-17B. J Immunol 142, 3134-3139 (1989)
¹³⁹D. M. Kania, N. Binkley, M. Checovich, T. Havighurst, M. Schilling & W. B. Ershler: Elevated plasma levels of interleukin-6 in postmenopausal women do not correlate with bone density. J Am Geriat Soc 43, 236-239 (1995)
¹⁴⁰S. H. Ralston: Analysis of gene expression in human bone biopsies by polymerase chain reaction: evidence for enhanced Cytokine expression in post-menopausal osteoporosis. J Bone Miner Res 9, 883-890 (1994)
¹⁴¹R. A. Daynes, B. A. Araneo, W. B. Ershler, C. Maloney, G. Z. Li & S. Y. Ryu: Altered regulation of IL-6 production with normal aging. Possible linkage to the age-associated decline in dehydroepiandrosterone and its sulfated derivative. J Immunol 150, 5219-5230 (1993)
¹⁴²R. Pacifici, C. Brown, E. Puscheck, E. Friedrich, E. Slatopolsky, D. Maggio, R. McCracken & L. V. Avioli: Effect of surgical menopause and estrogen replacement on Cytokine release from human blood mononuclear cells. Proc Natl Acad Sci USA 88, 5134-5138 (1991)
¹⁴³G. Pioli, G. Basini, M. Pedtazzoni, G. Musetti, V. Ulietti, D. Bresciani, P. Villa, A Bacchi, D. Hughes, M. Russel & M. Passeri: Spontaneous release of interleukin-1 and interleulin-6 by peripheral blood mononuclear cells after oophorectomy. Clin Sci 83, 503-507 (1992) G. Girasole, R. L. Jilka, F. Passeri, S. Boswell, G. Boder, D. C. Williams & S. C. Manologas: 17β-Estradiol inhibits interleulin-6 production by bone marrow-derived stromal cells and osteoblasts in vitro: a potential mechanism for the antiosteoporotic effect of estrogens. J Clin Invest 89, 883-891 (1992)
¹⁴⁵G. Girasole, R. L. Jilka, F. Passeri, S. Boswell, G. Boder, D. C. Williams & S. C. Manologas: 17β-Estradiol inhibits interleukin-6 production by bone marrow-derived stromal cells and osteoblasts in vitro: a potential mechanism for the antiosteoporotic effect of estrogens. J Clin Invest 89, 883-891 (1992)
¹⁴⁶R. L. Jilka, C. Hangoc, G. Girasole, G. Passeri, D. C. Williams, J. S. Abrams, B. Boyce, H. Broxmeyer & S. C. Manolagas: Increased osteoclast development after estrogen loss: Mediation by interleukin-6. Science 257, 88-91 (1992)
¹⁴⁷Chauhan, D., Uchiyama, H., Akbarali, Y., Urashima, M., Yamamoto, K, Libermann, T. A & Anderson, K. C. (1996) Multiple myeloma cell adhesion-induced interleukin-6 expression in bone marrow stromal cells involves activation of NF-kappa B. Blood 87, 1104-1112.
¹⁴⁸Paget J. On a form of chronic inflammation of bone (osteitis deformans). Med Chir Trans. 1877;60:37.
¹⁴⁹Krane S. Paget's disease of bone. Calcif Tissue Int. 1986;38:309-317.
¹⁵⁰Meunier P J, Salson C, Mathieu L, et al. Skeletal distribution and biochemical parameters of Paget's disease. Clin Orthop. 1987;217:37-44.
¹⁵¹Rodman C. D. Paget's Disease of the bone: Which patients need treatment? Women Health Primary Care 1998;1(3):261-272
¹⁵²Roodman G D. Osteoclast function in Paget's disease and multiple myeloma. Bone. 1995;17:57S-61S.
¹⁵³Lew D P, Waldvogel F A: Osteomyelitis. N Engl J Med 1997, 336:999-1007
¹⁵⁴Bost K L, Ramp W K, Nicholson N, Bento J L, Marriott I, Hudson M C: Staphylococcus aureus infection of mouse or human osteoblasts induces high levels of IL-6 and IL-12 production. J Infect Dis 1999, 180:1912-1920
¹⁵⁵Bost K L, Bento J L, Ellington J K, Marriott I, Hudson M C: Induction of colony stimulating factor expression following Staphylococcus or Salmonella interaction with mouse or human osteoblasts. Infect Immun 2000, 68:5075-5083
¹⁵⁶Am J Pathol. 2004 April;164(4):1399-406. Osteoblasts express the inflammatory cytokine interleulin-6 in a murine model of Staphylococcus aureus osteomyelitis and infected human bone tissue. Marriott I, Gray D L, Tranguch S L, Fowler V G Jr, Stryjewski M, Scott Levin L, Hudson M C, Bost K L.
¹⁵⁷ Trans R Soc Trop Med Hyg. 2000 March-April;94(2):221-4. Local and systemic concentrations of tumour necrosis factor-alpha, interleukin-6 and interleukin-8 in bacterial osteomyelitis. Fullilove S, Jellis J, Hughes S P, Remick D G, Friedland J S of tumour
¹⁵⁸Enitza D. George, M.D., and Richard Sadovsky, M.D., M.S. Multiple Myeloma: Recognition and Management. American Academy of Family Physicians.
¹⁵⁹Ravaud P, Thepot C, Auleley G R, Amor B. Imaging of multiple myeloma. Ann Med Interne [Paris] 1996;147:370-5.
¹⁶⁰Radiologe 1992 October;32(10):509-15 Bone marrow edema in MRT Vahlensieck M, Reiser M.
¹⁶¹Teoh G, Anderson K C. Interaction of tumor and host cells with adhesion and extracellular matrix molecules in the development of multiple myeloma. Hematol Oncol Clin North Am 1997; 11:27-42
¹⁶²Omoigui S. The Biochemical Origin of Pain: How a new law and new drugs have led to a medical breakthrough in the treatment of Persistent Pain. State of the Art Technologies 2004
¹⁶³Jeannette E. South-Paul, M.D. Osteoporosis: Part I. Evaluation and Assessment. American Family Physician. Mar. 1, 2001
¹⁶⁴Harper K D, Weber T J. Secondary osteoporosis. Diagnostic considerations. Endocrinol Metab Clin North Am 1998;27(2):325-48
¹⁶⁵P. Navarra, S. Tsagarakis, M. Faria, L. H. Rees, M. Besser & A. B. Grossman: Interleukin-1 and -6 stimulate the release of corticotropoin-releasing hormone from rat hypothalamus in vitro via eicosanoid cyclooxygenase pathway. Endocrinology 128, 37-44 (1990)
¹⁶⁶ K. Lyson, K. Milenkovic & S. M. McCann: The stimulatory effect of interleulin-6 on corticotorpinreleasing factor and thyrotropin-releasing hormone secretion in vitro. Prog Neuroendocrinol Immunol 4, 161-165 (1991)
¹⁶⁷Spine 2000 December 1;25(23):2975-80 2000 Volvo Award winner in basic science studies: Exogenous tumor necrosis factor-alpha mimics nucleus pulposus-induced neuropathology. Molecular, histologic, and behavioral comparisons in rats. Igarashi T, Kikuchi S, Shubayev V, Myers R R
¹⁶⁸Spine 2001 April 15;26(8):863-9 Selective inhibition of tumor necrosis factor-alpha prevents nucleus pulposus-induced thrombus formation, intraneural edema, and reduction of nerve conduction velocity: possible implications for future pharmacologic treatment strategies of sciatica. Olmarker K, Rydevik B.
¹⁶⁹Schmerz 2001 December;15(6):425-9 Radicular pain—recent pathophysiologic concepts and therapeutic implications. Olmarker K.
¹⁷⁰J Orthop Res 2000 September;18(5):815-20 Nitric oxide as a mediator of nucleus pulposus-induced effects on spinal nerve roots. Brisby H, Byrod G, Olmarker K, Miller VM, Aoli Y, Rydevik B.
¹⁷¹Spine 1997 November 1;22(21):2484-8 The inflammatory properties of contained and noncontained lumbar disc herniation. Nygaard O P, Mellgren S I, Osterud B.
¹⁷²Environ Health Perspect 1995 January;103(1):54-6 Neurogenic switching: a hypothesis for a mechanism for shifting the site of inflammation in allergy and chemical sensitivity. Meggs W J.
¹⁷³BioDrugs 2001;15(8):521-31 Neuroendocrine and immune aspects of fibromyalgia. van West D, Maes M.
¹⁷⁴Arthritis Rheum 1994 November;37(11):1593-601 Elevated cerebrospinal fluid levels of substance P in patients with the fibromyalgia syndrome. Russell I J, Orr MD, Littman B, Vipraio G A, Alboukrek D, Michalek J E, Lopez Y, MacKillip F.
¹⁷⁵Pain 1988 January;32(1):21-6 Elevated CSF levels of substance P and high incidence of Raynaud phenomenon in patients with fibromyalgia: new features for diagnosis. Vaeroy H, HeHe R, Forre O, Kass E, Terenius L.
¹⁷⁶J Rheumatol Suppl 1989 November;19:94-7 Modulation of pain in fibromyalgia (fibrositis syndrome): cerebrospinal fluid (CSF) investigation of pain related neuropeptides with special reference to calcitonin gene related peptide (CGRP). Vaeroy H, Sakurada T, Forre O, Kass E, Terenius L.
¹⁷⁷ Z Rheumatol 1998;57 Suppl 2:63-6 Neurochemical pathogenesis of fibromyalgia. Russell I J.
¹⁷⁸Pain 2000 August;87(2):201-11 Changes in the concentrations of amino acids in the cerebrospinal fluid that correlate with pain in patients with fibromyalgia: implications for nitric oxide pathways. Larson A A, Giovengo S L, Russell I J, Michalek J E.
¹⁷⁹Rheumatology (Oxford) 2001 July;40(7):743-9 Cytokines play an aetiopathogenetic role in fibromyalgia: a hypothesis and pilot study. Wallace D J, Linker-Israeli M, Hallegua D, Silverman S, Silver D, Weisman M H.
¹⁸⁰J Urol 2000 December;164(6):2119-25 Pentosanpolysulfate inhibits mast cell histamine secretion and intracellular calcium ion levels: an alternative explanation of its beneficial effect in interstitial cystitis. Chiang G, Patra P, letourneau R, Jeudy S, Boucher W, Green M, Sant G R, Theoharides T C.
¹⁸¹Br J Urol 1995 June;75(6):744-50 Increased number of substance P positive nerve fibres in interstitial cystitis. Pang X, Marchand J, Sant G R, Kream R M, Theoharides T C.
¹⁸²J Urol 1998 August;160(2):605-11 Comment in: J Urol 1998 August;160(2):298 High affinity binding sites for [3H] substance P in urinary bladders of cats with interstitial cystitis. Buffington C A, Wolfe S A Jr.
¹⁸³J Urol 1992 March;147(3):587-91 Interstitial cystitis: increased sympathetic innervation and related neuropeptide synthesis. Hohenfellner M, Nunes L, Schmidt R A, Lampel A, Thuroff J W, Tanagho E A.
¹⁸⁴ J Urol 1996 August;156(2 Pt 1):532-5 Potentiation by bradykinin and substance P of purinergic neurotransmission in urinary bladder. Patra P B, Westfall D P.
¹⁸⁵Urology 1999 September;54(3):450-3 Preliminary study on urinary cytokine levels in interstitial cystitis: does intravesical bacille Calmette-Guerin treat interstitial cystitis by altering the immune profile in the bladder? Peters K M, Diokno A C, Steinert B W.
¹⁸⁶ Can J Neurol Sci 1999 November;26 Suppl 3:S12-9 Pathophysiology of migraine—new insights. Hargreaves R J, Shepheard S L.
¹⁸⁷Blood Vessels 1991;28(1-3):35-45 Innervation and effects of dilatory neuropeptides on cerebral vessels. New aspects. Edvinsson L
¹⁸⁸Microsc Res Tech 2001 May 1;53(3):167-78 Neurogenic inflammation in the context of migraine. Williamson D J, Hargreaves R J.
¹⁸⁹Lakartidningen 2001 September 26;98(39):4176-83 [Both neurogenic and vascular causes of primary headache] [Article in Swedish] Edvinsson L.
¹⁹⁰Cerebrovasc Brain Metab Rev 1989 Fall;1(3):230-52 Neuropeptides in the cerebral circulation. Uddman R, Edvinsson L.
¹⁹¹ Regulation of meningeal blood flow by neuropeptides: Relevance to migraine By K. Messlinger, M. Pawlak
¹⁹²Pain 2000 December 1;88(3):239-48 Possible role of inflammatory mediators in tactile hypersensitivity in rat models of mononeuropathy. Cui J G, Holmin S, Mathiesen T, Meyerson B A, Linderoth B.
¹⁹³Eur J Neurosci 1999 March;11(3):837-46 Adrenergic innervation of rat sensory ganglia following proximal or distal painful sciatic neuropathy: distinct mechanisms revealed by anti-NGF treatment. Ramer M S, Bisby M A.
¹⁹⁴ Neurosci Lett 2002 February 22;319(3):137-40 Increases in the concentration of brain derived neurotrophic factor in the lumbar spinal dorsal horn are associated with pain behavior following chronic constriction injury in rats. Miletic G, Miletic V.
¹⁹⁵Clin J Pain 2000 June;16(2Suppl):S7-11 Neurotrophic factors and pain. Apfel S C.
¹⁹⁶ J Hand Surg [Am] 2001 July;26(4):635-44 Nerve growth factor inhibition prevents traumatic neuroma formation in the rat. Kryger G S, Kryger Z, Zhang F, Shelton D L, Lineaweaver W C, Buncke H J.
¹⁹⁷Pain 1999 February;79(2-3):265-74 Effect of NGF and anti-NGF on neuropathic pain in rats following chronic constriction injury of the sciatic nerve. Ro I S, Chen S T, Tang L M, Jacobs J M.
¹⁹⁸Clin J Pain 2000 June;16(2 Suppl):S12-20 Peripheral neuropathic pain: from mechanisms to symptoms. Baron R.
¹⁹⁹Clin J Pain 1991;7 Suppl 1:S8-15 Pathophysiological mechanisms of fibromyalgia. Zimmermann M.
²⁰⁰Neuroreport 2001 July 20;12(10):2079-84 NF-kappa B decoy suppresses cytokine expression and thermal hyperalgesia in a rat neuropathic pain model. Sakaue G, Shimaoka M, Fukuoka T, Hiroi T, Inoue T, Hashimoto N, Sakaguchi T, Sawa Y, Morishita R, Kiyono H, Noguchi K, Mashimo T.
²⁰¹ Nature 410, 471-475 (2001) Interleukin-l-mediated induction of Cox-2 in the CNS contributes to inflammatory pain hypersensitivity Tarek A. Samad, Kimberly A. Moore, Adam Sapirstein, Sara Billet, Andrew Allchorne, Stephen Poole, Joseph V. Bonventre & Clifford J. Woolf
²⁰²Oyelese, A A., Rizzo M. A, Waxman, S. G. and Kocsis, J. D. (1997) Differential effects of NGF and BDNF on axotomy-induced changes in GABAA receptor-mediated conductance and sodium currents in cutaneous afferent neurons. Journal of Neurophysiology, 78:31-42.
²⁰³Waxman, S. G. (1999) The molecular pathophysiology of pain: abnormal expression of sodium channel genes and its contributions to hyperexcitability in primary sensory neurons. Pain. 6, S133-S140.
²⁰⁴Black, J. A, langworthy, K., Hinson, A. W., Dibb-Hajj, S. D., and Waxman, S. G. (1997) NGF has opposing effects on Na+ channel III and SNS gene expression in spinal sensory neurons. NeuroReport, 8: 2331-2335.
²⁰⁵Everill, B., and Kocsis, J. D., (1999) Reduction of potassium currents in identified cutaneous afferent DRG neurons after axotomy. Journal of Neurophysiology, 82, 700-708.
²⁰⁶Lancet 1993 October 23;342(8878):1012-6 Signs and symptoms of reflex sympathetic dystrophy: prospective study of 829 patients. Veldman P H, Reynen H M, Arntz I E, Goris R J.
²⁰⁷Radiologe 1992 October;32(10):509-15 [Bone marrow edema in MRT] [Article in German] Vahlensieck M, Reiser M.
²⁰⁸Pain 2000 December 1;88(3):259-66 Patterns of spread in complex regional pain syndrome, type I (reflex sympathetic dystrophy). Maleki J, LeBel A A, Bennett G J, Schwartzman R J.
²⁰⁹Unfallchirurg 1997 February;100(2):90-9 [Sudeck's syndrome. Was Sudeck right?] [Article in German] van der Laan L, Goris R J.
²¹⁰Vasc Med 1998;3(3):207-14 Reflex sympathetic dystrophy: facts and hypotheses. Kurvers H A.
²¹¹Pharmacol Rev 2000 December;52(4):595-638 The sympathetic nerve—an integrative interface between two supersystems: the brain and the immune system. Elenkov I J, Wilder R I, Chrousos G P, Vizi E S.
²¹² Neurology 2001 December 26;57(12):2179-84 The important role of neuropeptides in complex regional pain syndrome. Birklein F, Schmelz M, Schifter S, Weber M.
²¹³Acta Orthop Belg 1998 December;64(4):448-51 Role of neuropeptides in pathogenesis of reflex sympathetic dystrophy. Blair S J, Chinthagada M, Hoppenstehdt D, Kijowski R, Fareed J.
²¹⁴Hyperalgesia from subcutaneous cytokines By Stephen Poole, Fernando de Queiroz Cunha and Sergio Henriques Ferreira
²¹⁵Rheumatology (Oxford) 2001 September;40(9):995-1001 Interleukln-1-induced subacromial synovitis and shoulder pain in rotator cuff diseases. Gotoh M, Hamada K, Yamakawa H, Yanagisawa K, Nakamura M, Yamazaki H, Ueyama Y, Tamaoki N, Inoue A, Fukuda H
²¹⁶J Orthop Res 1999 May;17(3):392-7 Increased interleukin-1beta production in the synovium of glenohumeral joints with anterior instability. Gotoh M, Hamada K, Yamakawa H, Nakamura M, Yamazaki H, Inoue A, Fukuda H.
²¹⁷Kobe J Med Sci 2001 February;47(1):25-34 Immunolocalization of cytokines and growth factors in subacromial bursa of rotator cuff tear patients. Sakai H, Fujita K, Sakai Y, Mizuno K/
²¹⁸Am J Obstet Gynecol 1991 October;165(4 Pt 2):1228-33 Vulvar vestibulitis syndrome: an overview. Marinoff S C, Turner M L
²¹⁹Obstet Gynecol 1997 February;89(2):291-6 Elevated tissue levels of interleukin-1 beta and tumor necrosis factor-alpha in vulvar vestibulitis. Foster D C, Hasday J D
²²⁰ Eur J Biochem 1987 June 15;165(3):675-81 Botulinum toxin A blocks glutamate exocytosis from guinea-pig cerebral cortical synaptosomes. Sanchez-Prieto J, Sihra T S, Evans D, Ashton A, Dolly J O, Nicholls D G.
²²¹J Biol Chem 1993 May 25;268(15):11057-64 Botulinum toxin inhibits arachidonic acid release associated with acetylcholine release from PC12 cells. Ray P, Berman J D, Middleton W, Brendle J.
²²²Cell Tissue Res 1992 February;267(2):291-300 Vasoactive intestinal polypeptide and acetylcholine coexist with neuropeptide Y, dopamine-beta-hydroxylase, tyrosine hydroxylase, substance P or calcitonin gene-related peptide in neuronal subpopulations in cranial parasympathetic ganglia of rat. Hardebo J E, Suzuki N, Ekblad E, Owman C.
²²³Mol Cell Biol 1989 May;9(5):2239-43 Effects of botulinum toxin type D on secretion of tumor necrosis factor from human monocytes. Imamura K Spriggs D, Ohno T, Kufe D.
²²⁴ Arthritis Rheum 2001 September;44(9):2112-7 Efficacy of etanercept in the treatment of the entheseal pathology in resistant spondylarthropathy: a clinical and magnetic resonance imaging study. Marzo-Ortega H, McGonagle D, O'Connor P, Emery P.
²²⁵J Peripher Nerv Syst 2001 June;6(2):67-72 Etanercept reduces hyperalgesia in experimental painful neuropathy. Sommer C, Schafers M, Marziniak M, Toyka K V.
²²⁶Spine 2001 April 15;26(8):863-9 Selective inhibition of tumor necrosis factor-alpha prevents nucleus pulposus-induced thrombus formation, intraneural edema, and reduction of nerve conduction velocity: possible implications for future pharmacologic treatment strategies of sciatica. Olmarker K, Rydevik B.
²²⁷Clin Exp Rheumatol. 2002 May-Jun;20(3):359-64. Clodronate treatment reduces serum levels of interleukin-6 soluble receptor in Paget's disease of bone. Rendina D, Postiglione L, Vuotto P, Numis FG, Di Domenico G, Viceconti R, Mossetti G, Nunziata V
²²³Methods Find Exp Clin Pharmacol. 1999 October;21(8):519-22. Etidronate inhibits the production of IL-6 by osteoblast-like cells. Olmos J M, De Vega T, Perera L, Riancho J A, Amado J A, Gonzalez-Macias J. Departamento de Medicina Intema, Hospital Marques de Valdecilla, Universidad de Cantabria, Santander, Spain
²²⁹Scand J Rheumatol. 1998;27(1):38-41. Bisphosphonates inhibit IL-6 production by human osteoblast-like cells. Giuliani N, Pedrazzoni M, Passeri G, Girasole G. Istituto di Clinica Medica Generale, University of Parma, Italy.
²³⁰Endocrinology. 1996 June;137(6):2324-33. Bisphosphonates induce osteoblasts to secrete an inhibitor of osteoclast-mediated resorption. Vitte C, Fleisch H, Guenther H L.
²³¹J Clin Invest. 1999 November;104(10):1363-74. Prevention of osteocyte and osteoblast apoptosis by bisphosphonates and calcitonin. Plotkin L I, Weinstein R S, Parfitt A M, Roberson P K, Manolagas S C, Bellido T
²³² Eur J Haematol. 1998 August;61(2):128-34. Long-term oral pamidronate treatment inhibits osteoclastic bone resorption and bone turnover without affecting osteoblastic function in multiple myeloma. Abildgaard N, Rungby J, Glerup H, Brixen K, Kassem M, Brincker H, Heickendorff L, Eriksen E F, Nielsen J L.
²³³Cancer Treat Rev. 2003 June;29(3):189-98. Pathophysiology of bone metastases from prostate cancer and the role of bisphosphonates in treatment. Eaton C L, Coleman R E
²³⁴Br J Cancer. 2003 July 7;89(1):178-84. Bisphosphonates antagonise bone growth factors' effects on human breast cancer cells survival. Frornigue O, Kheddoumi N, Body J J
²³⁵Endocrinology. 1998 March;139(3):1338-45. Regulation of matrix metalloproteinases (MMP-2, -3, -9, and -13) by interleukin-1 and interleukin-6 in mouse calvaria: association of MMP induction with bone resorption. Kusano K, Miyaura C, Inada M, Tamura T, Ito A, Nagase H, Kamoi K, Suda T.
²³⁶Drugs Today (Barc). 2002 February;38(2):91-102. The role of osteoclastic activity in prostate cancer skeletal metastases. Keller E T.
²³⁷J Orthop Res. 2003 January;21(1):81-7. Effect of pamidronate on the stimulation of macrophage TNF-alpha release by ultra-high-molecular-weight polyethylene particles: a role for apoptosis. Huk O L, Zukor D J, Antoniou J, Petit A
²³⁸Arthritis Rheum. 2002 March;46(3):785-92. Tumor necrosis factor alpha-mediated joint destruction is inhibited by targeting osteoclasts with osteoprotegerin. Redlich K, Hayer S, Maier A, Dunstan C R, Tohidast-Akrad M, Lang S, Turk B, Pietschmann P, Woloszczuk W, Haralambous S, Kollias G, Steiner G, Smolen J S, Schett G.
²³⁹J Rheumatol. 1999 November;26(11):2318-23. Evaluation of bone turnover and osteoclastic cytokines in early rheumatoid arthritis treated with alendronate. Cantatore F P, Acquista C A, Pipitone V.
²⁴⁰Eur J Haematol. 1998 August;61(2):128-34. Long-term oral pamidronate treatment inhibits osteoclastic bone resorption and bone turnover without affecting osteoblastic function in multiple myeloma. Abildgaard N, Rungby J, Glerup H, Brixen K, Kassem M, Brincker H, Heickendorff L, Eriksen E F, Nielsen J L
²⁴¹J Bone Miner Res. 1995 October;10(10):1478-87. Bisphosphonates promote apoptosis in murine osteoclasts in vitro and in vivo. Hughes D E, Wright K R, Uy H L, Sasaki A, Yoneda T, Roodman G D, Mundy G R, Boyce B F.
²⁴²Cancer 1997 October 15;80(8 Suppl):1546-56 Mechanisms of bone metastasis. Mundy G R.
²⁴³Clin Rheumatol 1997 January;16(1):51-6 Treatment of severe, recalcitrant reflex sympathetic dystrophy: assessment of efficacy and safety of the second generation bisphosphonate pamidronate. Cortet B, Flipo R M, Coquerelle P, Duquesnoy B, Delcambre B.
²⁴⁴J Interferon Cytokine Res. 2003 November;23(11):649-54. Fever after zoledronic acid administration is due to increase in TNF-alpha and IL-6. Dicuonzo G, Vincenzi B, Santini D, Awisati G, Rocci L, Battistoni F, Gavasci M, Borzomati D, Coppola R, Tonini G.
²⁴⁵Hematology (Am Soc Hematol Educ Program) 2000;:147-165 Multiple Myeloma: New Insights and Therapeutic Approaches. Anderson K C, Kyle R A, Dalton W S, Landowski T, Shain K, Jove R, Hazlehurst L, Berenson J.
²⁴⁶Lab Invest 2001 August;81(8):1109-17 Inhibitors of Phosphodiesterase Isoforms III or IV Suppress Islet-Cell Nitric Oxide Production. Beshay E, Prudhomme G J.
²⁴⁷Spine 2001 April 15;26(8):870-5 Prevention of compartment syndrome in dorsal root ganglia caused by exposure to nucleus pulposus. Yabuli S, Onda A, Kikuchi S, Myers R R.
²⁴⁸Clin Exp Immunol 1996 June;104(3):501-8 Inhibitory effect of clarithromycin on costimulatory molecule expression and cytokine production by synovial fibroblast-like cells. Matsuoka N, Eguchi K, Kawakami A, Tsuboi M, Kawabe Y, Aoyagi T, Nagataki S.
²⁴⁹Amin A R, Attur M G, Thakker G D, Patel P D, Vyas P R, Patel R N, Patel I R, Abramson S B A novel mechanism of action of tetracyclines: effects on nitric oxide syntheses Proc Natl Acad Sci U S A 1996; 93: 14014-9.
²⁵⁰Steinrmeyer J, Daufeldt S, Taiwo Y O Pharmacological effect of tetracyclines on proteoglycanases from interleukin-l-treated articular cartilage, Biochem Pharmacol 1998; 55: 93-100
²⁵¹Kloppenburg M, Verweij C L, Miltenburg A M, Verboeven A J, Daha M R, Dijlanans B A, Breeveld F C, The influence of tetracyclines on T cell activation Clin EU Immunol 1995; 102: 635-41.
²⁵²Shapira L, Houri Y, Barak V, Soskolne W A, Halabi A, Stabholz A Tetracycline inhibits' Porphyromonas gingivalis lipopolysaccharide-induced lesions in vivo and TNF .alpha. processing in vitro J Periodontal Res 1997; 32: 183-8
²⁵³Chao C C, Hu S, Ehrlich L, Peterson P K Interleukin-1 and tumor necrosis factor-alpha synergistically mediate neurotoxicity: involvement of nitric oxide and of N-methyl-D-aspartate receptors Brain Behav Immun 1995; 9: 355-65
²⁵⁴ Bluthe R M, Dantzer R, Kelley K W Interleukin-I mediates behavioural but not metabolic effects of tumor necrosis factor alpha in mice Eur J Pharmacol 1991; 209: 281-3.
²⁵⁵.Hartung B P, Jung S, Stoll G, Zielasek J, Schmidt B, Archelos J J, Toyka K V Inflammatory mediators in demyelinating disorders of the CNS and PNS J Neuroinununol 1992; 40: 197-210.
²⁵⁶Sommer C, Schmidt C, George A, Toyka K V A metalloprotease-inhibitor reduces pain associated behaviour in mice with experimental neuropathy Neurosci Lett 1997;237:45-8.
²⁵⁷Stoll G, Jung S, Jander S, van der Meide P, Hartung B P Tumor necrosis factor-alpha in immunomediated demyelination and Wallerian degeneration of the rat peripheral nervous system Neuroimmunol 1993; 45: 175-82.
²⁵⁸J Rheumatol 1998 March;25(3):532-5 Oral administration of doxycycline reduces collagenase and gelatinase activities in extracts of human osteoarthritic cartilage. Smith G N Jr, Yu L P Jr, Brandt K D, Capello W N.
²⁵⁹Ryumachi 1998 December;38(6):801-9 [An evaluation of efficacy of minocycline as an anti-rheumatic drug in patients with active and refractory rheumatoid arthritis] [Article in Japanese] Kawanaka N, Yamamura M, Hashimoto H, Okamoto H, Morita Y, Kawashima M, Aita T, Okamoto A, Makino H.
²⁶⁰ Arthritis Rheum 1999 August;42(8):1691-5 Treatment of early seropositive rheumatoid arthritis with minocycline: four-year followup of a double-blind, placebo-ontrolled trial. O'Dell J R, Paulsen G, Haire C E, Blakely K, Palmer W, Wees S, Eckhoff P J, Klassen L W, Churchill M, Doud D, Weaver A, Moore G F.
²⁶¹Spine 2000 December 1;25(23):2975-80 2000 Volvo Award winner in basic science studies: Exogenous tumor necrosis factor-alpha mimics nucleus pulposus-induced neuropathology. Molecular, histologic, and behavioral comparisons in rats. Igarashi T, Kikuchi S, Shubayev V, Myers R R
²⁶²Spine 1998 December 1;23(23):2538-44 Tumor necrosis factor alpha and nucleus-pulposus-induced nerve root injury. Olmarker K, Larsson K.
²⁶³Scand J Rheumatol Suppl 2000;113:37-45 Preclinical and clinical pharmacology of the 5-HT3 receptor antagonists. Wolf H.
²⁶⁴Free Radic Res Commun 1987;3(1-5):13-8 Are toxic oxygen radicals involved in the pathogenesis of reflex sympathetic dystrophy? Goris R J, Dongen L M, Winters H A.
²⁶⁵Anesthesiology 1995 November;83(5):1046-54 Intrathecal amitriptyline acts as an N-methyl-D-aspartate receptor antagonist in the presence of inflammatory hyperalgesia in rats. Eisenach J C, Gebhart G F.
²⁶⁶Psychoneuroendocrinology 2000 November;25(8):785-97 The effect of repeated amitriptyline and desipramine administration on cytokine release in C57BL/6 mice. Kubera M, Holan V, Mathison R, Maes M.
²⁶⁷Eur J Neurosci 2000 July;12(7):2623-33 Inflammatory cytokines and related genes are induced in the rat hippocampus by limbic status epilepticus. De Simoni M G, Perego C, Ravizza T, Moneta D, Conti M, Marchesi F, De Luigi A, Garattini S, Vezzani A.
²⁶⁸Neuroscience 1999;91(1):211-9 Corticosterone and phenytoin reduce neuronal nitric oxide synthase messenger RNA expression in rat hippocampus. Reagan L P, McKittrick C R, McEwen B S.
²⁶⁹Res Commun Mol Pathol Pharmacol 1999;105(1-2):23-33 Zonisamide inhibits nitric oxide synthase activity induced by N-methyl-D-aspartate and buthionine sulfoximine in the rat hippocampus. Noda Y, Mori A, Packer L
²⁷⁰Brain Res 2000 February 28;857(1-2):246-51 Sodiun valproate inhibits production of TNF-alpha and IL-6 and activation of NF-kappaB. Ichiyama T, Okada K, Lipton J M, Matsubara T, Hayashi T, Furukawa S.
²⁷¹Am J Med Sci 1994 July;308(1):38-40 Positive response to oral divalproex sodium (Depakote) in patients with spasticity and pain. Zachariah S B, Borges E F, Varghese R, Cruz A R, Ross G S.
²⁷²Pain 2000 December 1;88(3):267-75 Thalidomide treatment in chronic constrictive neuropathy decreases endoneurial tumor necrosis factor-alpha, increases interleukin-10 and has long-term effects on spinal cord dorsal horn met-enkephalin. George A, Marziniak M, Schafers M, Toyka K V, Sommer C.
²⁷³Arch Intern Med 2001 Nov 12;161(20):2502-3 Complete resolution of reflex sympathetic dystrophy with thalidomide treatment. Rajkumar S V, Fonseca R, Witzig T E.
²⁷⁴Atherosclerosis. 2003 August;169(2):283-91. Soluble intercellular adhesion molecule-1 and interleukin-6 levels reflect endothelial dysfunction in patients with primary hypercholesterolaemia treated with atorvastatin. Nawawi H, Osman N S, Annuar R, Khalid B A, Yusoff K.
²⁷⁵Osteoporos Int. 2003 June;14(4):273-82. Epub 2003 May 08 HMG CoA reductase inhibitors and the skeleton: a comprehensive review. Bauer D C.
²⁷⁶J Clin Densitom. 2002 Summer;5(2):151-8. Effect of HMG-COA reductase inhibitors (statins) on bone mineral density. Funkhouser H L, Adera T, Adler R A.
²⁷⁷ Arch Intern Med. 2002 March 11;162(5):537-40. Statin use, bone mineral density, and fracture risk: Geelong Osteoporosis Study. Pasco J A, Kotowicz M A, Henry M J, Sanders K M, Nicholson G C; Geelong Osteoporosis Study.
²⁷⁸ J Bone Miner Res. 2003 January;18(1):88-96. The ability of statins to inhibit bone resorption is directly related to their inhibitory effect on HMG-COA reductase activity. Staal A, Frith J C, French M H, Swartz J, Gungor T, Harrity T W, Tamasi J, Rogers M J, Feyen J H
²⁷⁹Pain 1999 April;80(3):555-66 Methylprednisolone prevents the development of autotomy and neuropathic edema in rats, but has no effect on nociceptive thresholds. Kingery W S, Castellote J M, Maze M.
²⁸⁰Immunol Lett 1997 August;58(3):153-7 Chronic exposure to morphine, but not ethanol, attenuates the expression of interleukin-1 beta converting enzyme in rat spleen. Graf J A, Patel J A, Chang S L.
²⁸¹Clin Diagn Lab Immunol 1997 March;4(2):127-32 Immunoregulatory effects of morphine on human lymphocytes. Nair M P, Schwartz S A, Polasani R, Hou J, Sweet A, Chadha K C
²⁸² 36: J Neuroimmunol 1997 March;73(1-2):1-6 Effect of morphine on lipopolysaccharide-induced tumor necrosis factor-alpha production in vivo: involvement of the sympathetic nervous system. Bencsics A, Elenkov I J, Vizi E S.
²⁸³Int J Immunopharmacol 2000 August;22(8):603-14 Pharmacokinetics and cytokine production in heroin and morphine-treated mice. Pacifici R, di Carlo S, Bacosi A, Pichini S, Zuccaro P.
²⁸⁴Br J Pharmacol 2001 July;133(6):807-14 Role of opioid receptors in neurogenic dural vasodilation and sensitization of trigeminal neurones in anaesthetized rats. Williamson D J, Shepheard S L, Cook D A, Hargreaves R J, Hill R G, Cumberbatch M J.
²⁸⁵Br J Pharmacol 2001 July;133(6):807-14 Role of opioid receptors in neurogenic dural vasodilation and sensitization of trigeminal neurones in anaesthetized rats. Williamson D J, Shepheard S L, Cook D A, Hargreaves R J, Hill R G, Cumberbatch M J.
²⁸⁶ Eur J Neurosci 2000 July;12(7):2623-33 Inflammatory cytokines and related genes are induced in the rat hippocampus by limbic status epilepticus. De Simoni M G, Perego C, Ravizza T, Moneta D, Conti M, Marchesi F, De Luigi A, Garattini S, Vezzani A.
²⁸⁷ JAMA 1998 April 8;279(14):1076-82 Preemptive epidural analgesia and recovery from radical prostatectomy: a randomized controlled trial. Gottschalk A, Smith D S, Jobes D R, Kennedy S K, Lally S E, Noble V E, Grugan K F, Seifert H A, Cheung A, Malkowicz S B, Gutsche B B, Wein A J.
²⁸⁸ Acta Anaesthesiol Sin 1997 June;35(2):97-102 Erratum in: Acta Anaesthesiol Sin 1997 September;35(3):191 The preemptive effect of pre-incisional bupivacaine infiltration on postoperative analgesia following lower abdominal surgery under epidural anesthesia. Huang S J, Wang J J, Ho S T, Liu H S, Liaw W J, Li M J, Lu Y H.
²⁸⁹Arch Surg 1997 July;132(7):766-9 Comparing preincisional with postincisional bupivacaine infiltration in the management of postoperative pain. Bourget J L, Clark J, Joy N.

1. A method of the biochemical treatment of persistent pain disorders in a human or other animal subject, in accordance with Sota Omoigui's Law of Pain, which states that, the origin of all pain is inflammation and the inflammatory response. Said method comprises administering, to said subject, any one of the following combinations of components that are inhibitors of biochemical mediators of inflammation:
I. A and C
II. A and E
III. A, B, C and D
IV. A, B, C and E
V. A, B, D and E
VI. A, B, C, D and E
Wherein
A is a Prostaglandin inhibitor selected from the group consisting of corticosteroids, botulinum toxin, acetaminophen; ibuprofen; flurbiprofen; ketoprofen; naproxen; oxaprozin; etodolac; indomethacin; ketorolac; nabumetone; piroxicam; celecoxib; rofecoxib; meloxicam; JTE-522; L-745, 337; and NS398; or a pharmaceutically acceptable salt thereof.
B. is a modulator of neuronal transmission and an inhibitor of neuronal neuropeptide (substance p, glutamate, calcitonin gene related peptide, bradykinin, nitric oxide) release, selected from the group including of opioid analgesics, Ca(+) channel blockers, Na(+) channel blocKers, k(+) channel blockers, oxcarbazepine, zonisamide, lamotrigine, gabapentin, valproic acid, topiramate, carbamazepine, clonazepam, divalproex sodium, valproate sodium, an ester thereof, a pharmaceutically acceptable salt thereof, a hydrate thereof:
C. is an interleukin-1 (IL-1) inhibitor selected from the group including of interleukin 1 receptor antagonist (IL-1 RA), interleukin 1 receptor type II (IL-1R type II), monoclonal antibodies to interleukin 1 (including both chimeric and fully humanized forms), soluble receptors to interleukin 1, soluble receptors to interleukin 1 fused to an F.sub.c immunoglobulin fragment, bisphosphonates such as Pamidronate, Etidronate, Clodronate, Alendronate, phosphonic acid derivatives, an ester thereof, a pharmaceutically acceptable salt thereof, a hydrate thereof.
D. is an interleukin-6 (IL-6) inhibitor, selected from the group comprising monoclonal antibodies to IL-6, bisphosphonates such as Pamidronate, Etidronate, Clodronate, Alendronate, phosphonic acid derivatives, an ester thereof, a pharmaceutically acceptable salt thereof, a hydrate thereof
E. is a Tumor Necrosis Factor-Alpha (TNF-alpha) inhibitor selected from the group including of infliximab, adalimumab, etanercept, pegylated soluble TNF receptor Type I (PEGsTNF-R11), CDP571 (a humanized monoclonal anti-TNF-alpha antibody), D2E7 (a human anti-TNF mAb), Thalidomide based compounds, Tetracyclines, Pentoxifylline and Phosphodiesterase inhibitors.
said components being administered simultaneously or separately, in amounts which in combination have the effect of ameliorating the persistent pain disorder.

2. The method of claim 1, wherein;
a) said persistent pain disorder is musculoskeletal pain.
b) a therapeutically effective amount of said combinations of inhibitors of biochemical mediators of inflammation is administered subcutaneously, intramuscularly, intravenously, orally, rectally, by intra-articular, epidural or intrathecal injection.

3. The method of claim 1, wherein;
a) said persistent pain disorder is joint pain.
b) a therapeutically effective amount of said combinations of inhibitors of biochemical mediators of inflammation is administered subcutaneously, intramuscularly, intravenously, orally, rectally, by intra-articular, epidural or intrathecal injection.

4. The method of claim 1, wherein;
a) said persistent pain disorder is bone pain.
b) a therapeutically effective amount of said combinations of inhibitors of biochemical mediators of inflammation is administered subcutaneously, intramuscularly, intravenously, orally, rectally, by intra-articular, epidural or intrathecal injection.

5. The method of claim 1, wherein;
a) said persistent pain disorder is osteoarthritis or any other type of arthritis.
b) a therapeutically effective amount of said combinations of inhibitors of biochemical mediators of inflammation is administered subcutaneously, intramuscularly, intravenously, orally, rectally, by intra-articular, epidural or intrathecal injection.

6. The method of claim 1, wherein;
a) said persistent pain disorder is ligament or meniscus tear.
b) a therapeutically effective amount of said combinations of inhibitors of biochemical mediators of inflammation is administered subcutaneously, intramuscularly, intravenously, orally, rectally, by intra-articular, epidural or intrathecal injection.

7. The method of claim 1, wherein;
a) said persistent pain disorder is back or neck pain arising from injury to the nerve, muscle, joint, ligament or disk.
b) a therapeutically effective amount of said combinations of inhibitors of biochemical mediators of inflammation is administered subcutaneously, intramuscularly, intravenously, orally, rectally, by intra-articular, epidural or intrathecal injection.

8. The method of claim 1, wherein;
a) said persistent pain disorder is nerve pain including neurogenic inflammation, neuralgia, carpal tunnel syndrome, post herpetic neuralgia, phantom limb pain, vulvodynia.
b) a therapeutically effective amount of said combinations of inhibitors of biochemical mediators of inflammation is administered subcutaneously, intramuscularly, intravenously, orally, rectally, by intra-articular, epidural or intrathecal injection.

9. The method of claim 1, wherein;
a) said persistent pain disorder is neuropathic pain syndrome including neuralgia or nerve pain, carpal tunnel syndrome, post herpetic neuralgia, phantom limb pain, vulvodynia.
b) a therapeutically effective amount of said combinations of inhibitors of biochemical mediators of inflammation is administered subcutaneously, intramuscularly, intravenously, orally, rectally, by intra-articular, epidural or intrathecal injection.

10. The method of claim 1, wherein;
a) said persistent pain disorder is chronic regional pain syndrome also known as reflex sympathetic dystrophy.
b) a therapeutically effective amount of said combinations of inhibitors of biochemical mediators of inflammation is administered subcutaneously, intramuscularly, intravenously, orally, rectally, by intra-articular, epidural or intrathecal injection.

11. The method of claim 1, wherein;
a) said persistent pain disorder is fibromyalgia.
b) a therapeutically effective amount of said combinations of inhibitors of biochemical mediators of inflammation is administered subcutaneously, intramuscularly, intravenously, orally, rectally, by intra-articular, epidural or intrathecal injection.

12. The method of claim 1, wherein;
a) said persistent pain disorder is muscle pain
b) a therapeutically effective amount of said combinations of inhibitors of biochemical mediators of inflammation is administered subcutaneously, intramuscularly, intravenously, orally, rectally, by intra-articular, epidural or intrathecal injection.

13. The method of claim 1, wherein;
a) said persistent pain disorder is osteoporosis pain
b) a therapeutically effective amount of said combinations of inhibitors of biochemical mediators of inflammation is administered subcutaneously, intramuscularly, intravenously, orally, rectally, by intra-articular, epidural or intrathecal injection.

14. The method of claim 1, wherein;
a) said persistent pain disorder is bursitis including rotator cuff bursitis.
b) a therapeutically effective amount of said combinations of inhibitors of biochemical mediators of inflammation is administered subcutaneously, intramuscularly, intravenously, orally, rectally, by intra-articular, epidural or intrathecal injection.

15. The method of claim 1, wherein;
a) said persistent pain disorder is tendonitis.
b) a therapeutically effective amount of said combinations of inhibitors of biochemical mediators of inflammation is administered subcutaneously, intramuscularly, intravenously, orally, rectally, by intra-articular, epidural or intrathecal injection.

16. The method of claim 1, wherein;
a) said persistent pain disorder is soft tissue pain.
b) a therapeutically effective amount of said combinations of inhibitors of biochemical mediators of inflammation is administered subcutaneously, intramuscularly, intravenously, orally, rectally, by intra-articular, epidural or intrathecal injection.

17. The method of claim 1, wherein;
a) said persistent pain disorder is migraine.
b) a therapeutically effective amount of said combinations of inhibitors of biochemical mediators of inflammation is administered subcutaneously, intramuscularly, intravenously, orally, rectally, by intra-articular, epidural or intrathecal injection.

18. The method of claim 1, wherein;
a) said persistent pain disorder is interstitial cystitis.
b) a therapeutically effective amount of said combinations of inhibitors of biochemical mediators of inflammation is administered subcutaneously, intramuscularly, intravenously, orally, rectally, by intra-articular, epidural or intrathecal injection.

19. A method of treating bone density disorders in a human or other animal subject, in accordance with Sota Omoigui's Law of Pain, which states that, the origin of all pain is inflammation and the inflammatory response and the primary origin of osteoporosis is inflammation and the inflammatory response. Said method comprises administering, to said subject, any one of the following combinations of components that are inhibitors of biochemical mediators of inflammation:
I. A and B
II. A, B and C
Wherein
A is an interleukin-1 (IL-1) inhibitor selected from the group including of interleukin 1 receptor antagonist (IL-1 RA), interleukin 1 receptor type II (IL-1R type II), monoclonal antibodies to interleukin 1 (including both chimeric and fully humanized forms), soluble receptors to interleukin 1, soluble receptors to interleukin 1 fused to an F.sub.c immunoglobulin fragment, bisphosphonates such as Pamidronate, Etidronate, Clodronate, Alendronate, phosphonic acid derivatives, an ester thereof, a pharmaceutically acceptable salt thereof, a hydrate thereof.
B is an interleukin-6 (IL-6) inhibitor, selected from the group comprising monoclonal antibodies to IL-6, bisphosphonates such as Pamidronate, Etidronate, Clodronate, Alendronate, phosphonic acid derivatives, an ester thereof, a pharmaceutically acceptable salt thereof, a hydrate thereof, HMG-CoA reductase inhibitors such as lovastatin, simvastatin, pravastatin, fluvastatin, atorvastatin, rivastatin, red yeast rice, red yeast grain, red yeast powder and other statins or a pharmaceutically acceptable salt thereof.
C is a Tumor Necrosis Factor-Alpha (TNF-alpha) inhibitor selected from the group including of infliximab, adalimumab, etanercept, pegylated soluble TNF receptor Type I (PEGsTNF-R1), CDP571 (a humanized monoclonal anti-TNF-alpha antibody), D2E7 (a human anti-TNF mAb), Thalidomide based compounds, Tetracyclines, Pentoxifylline and Phosphodiesterase inhibitors.
said components being administered simultaneously or separately, in amounts which in combination have the effect of ameliorating the bone density disorder.

20. The method of claim 19, wherein;
a) said persistent bone density disorder is osteoporosis
b) a therapeutically effective amount of said combinations of inhibitors of biochemical mediators of inflammation is administered subcutaneously, intramuscularly, intravenously, orally, rectally, by intra-articular, epidural or intrathecal injection.

21. The method of claim 19, wherein;
a) said persistent bone density disorder is osteopenia
b) a therapeutically effective amount of said combinations of inhibitors of biochemical mediators of inflammation is administered subcutaneously, intramuscularly, intravenously, orally, rectally, by intra-articular, epidural or intrathecal injection.

22. The method of claim 19, wherein;
a) said persistent bone density disorder is inflammatory bone pain
b) a therapeutically effective amount of said combinations of inhibitors of biochemical mediators of inflammation is administered subcutaneously, intramuscularly, intravenously, orally, rectally, by intra-articular, epidural or intrathecal injection.
